(19) United States
(12) Patent Application Publication
(10) Pub. No.: US 2012/0115852 A1
(43) Pub. Date: May 10, 2012

(54) HETEROCYCLIC COMPOUNDS AS AUTOTAXIN INHIBITORS

(75) Inventors: Melanie Schultz, Darmstadt (DE); Kai Schiemann, Seeheim-Jugenheim (DE); Wolfgang Staehle, Ingelheim (DE)

(73) Assignee: Merck Patent Gesellschaft Mit Beschrankter Haftung, Darmstadt (DE)

(21) Appl. No.: 13/383,908

(22) PCT Filed: Jun. 17, 2010
(86) PCT No.: PCT/EP2010/003661

§ 371 Date: Jan. 13, 2012

(30) Foreign Application Priority Data
Jul. 16, 2009 (DE) 10 2009 033 392.4

(51) Int. Cl. A61K 31/551; C07D 401/12; A61P 35/02; C07D 487/04; A61P 35/00

(52) U.S. Cl. 514/220; 540/497; 544/360

(57) ABSTRACT

Compounds of the formula (I), in which R, R¹, R², X, X₁, Y, Y₁, Q, E, n1 and n2 have the meanings indicated in claim 1, are autotaxin inhibitors and can be employed for the treatment of tumours.

### BACKGROUND OF THE INVENTION

The invention was based on the object of finding novel compounds having valuable properties, in particular those which can be used for the preparation of medicaments.

The present invention relates to compounds and to the use of compounds for the treatment of diseases which are accompanied by an increase in the lysophosphatidic acid level, furthermore to pharmaceutical compositions which comprise these compounds.

In detail, the present invention relates to compounds of the formula I, which preferably inhibit one or more enzymes which regulate and/or modulate the lysophosphatidic acid (or LPA for short) level, to compositions which comprise these compounds, and to processes for the use thereof for the treatment of diseases and complaints, such as angiogenesis, cancer, tumour formation, growth and propagation, arteriosclerosis, ocular diseases, choroidal neovascularisation and diabetic retinopathy, inflammatory diseases, arthritis, neurodegeneration, restenosis, wound healing or transplant rejection. In particular, the compounds according to the invention are suitable for the therapy or prophylaxis of cancer diseases.

Autotaxin (ATX) is an enzyme which is responsible for the increase in the lysophosphatidic acid level in ascites and plasma (Xu et al. 1995, Clinical Cancer Research Vol. 1, page 1223 and Xu et al. 1995, Biochem. J. Vol-309, page 933). ATX converts lysophatidylcholine (LPC) into lysophosphatidic acid (Tokumura et al. 2002, J. Biol. Chem., Vol 277, page 39436 and Umezu-Gozo et al. 2002, J. Biol. Chem., Vol. 158, page 227) LPA is an intercellular lipid mediator which influences a multiplicity of biological and biochemical processes, such as, for example, smooth muscle contraction, thrombocyte aggregation and apoptosis (Tigyi et al. 2003 Prog. Lipid Res. Vol 42, page. 498 and Mills et al. 2003 Nat. Rev. Cancer Vol. 3, page 582 and Lynch et al. 2001 Prost. Lipid Med. Vol. 64, page 33). In addition, LPA can be found in increased concentrations in plasma and ascites fluid from ovarian cancer patients in the early and late phase. LPA plays a role there in tumour cell proliferation and invasion thereof into neighbouring tissue, which can result in metastasisation (Xu et al. 1995, Clinical Cancer Research Vol. 1, page 1223 and Xu et al. 1995, Biochem. J. Vol-309, page 933). These biological and phatobiological processes are switched on by the activation by LPA of G-protein-coupled receptors (Contos et al. 2000, Mol. Pharm. Vol 58, page. 1188).

For this reason, it is desirable to lower the LPA level for the treatment of tumour patients. This can be achieved by the inhibition of enzymes which are involved in LPA biosynthesis, such as, for example, autotaxin (ATX, Sano et al. 2002, J. Biol. Chem. Vol. 277, page 21197 and Aoki et al. 2003, J. Biol. Chem. Vol. 277 page 48737). Autotaxin belongs to the enzyme family of the nucleotides pyrophosphatases and phosphodiesterases (Goding et al. 1998, Immunol. Rev. Vol. 161, page 11) and represents an important starting point in antitumour therapy (Mills et al. 2003 Nat. Rev. Cancer Vol. 3, page 582 and Goto eta I. 2004 J. Cell. Biochem. Vol. 92, page 1115) since it is expressed to an increased extent in tumours and causes tumour cell proliferation and invasion into neighbouring tissue, which can result in metastases formation (Nam et al. 2000, Oncogene, Vol. 19 page 241). In addition, autotaxin together with other angiogenetic factors causes blood vessel formation in the course of angiogenesis (Nam et al. 2001, Cancer Res. Vol. 61 page. 6938). Angiogenesis is an important process in tumour growth, which ensures supply of the tumour with nutrients. For this reason, inhibition of angiogenesis is an important starting point in cancer and tumour therapy, with which the tumour can be starved to a certain extent (Folkman, 2007, Nature Reviews Drug Discovery Vol. 6, page 273-286).

Furthermore, autotaxin controls the migration of T cells into secondary lymphatic organs by means of the conversion of LPC into LPA. Naïve T cells constantly migrate between blood and secondary lymphatic organs, the lymph nodes, in the healthy organism. In order to migrate from the bloodstream into a lymph node, the T cells must overcome specialised blood vessels, so-called high endothelial venules (HEV). Autotaxin is involved in this process. HEV cells secrete autotaxin into the bloodstream. This binds to T cells and converts LPC into LPA on the surface thereof. LPA in turn binds to specific receptors on the surface of the T cells and increases their ability to migrate into lymph nodes. Treatment of T cells with an autotaxin mutant which is enzymatically inactive reduces their ability to migrate into lymph nodes [1]. Treatment of the T cells with the inhibitors developed by us can likewise block migration thereof into lymph nodes.

During inflammation, T cells can also migrate into other body tissue and drive forward the inflammation reaction there, which can result in organ damage. It has been shown in an animal model that blood vessels in inflamed tissue begin to express autotaxin [2]. It can therefore be assumed that autotaxin is also able to control the migration of T cells into body tissue during an inflammation. Increased autotaxin production is indeed also evident in humans both in inflamed intestinal tissue in the case of chronic inflammatory intestinal diseases [3] and also in affected joints [4] and synovial fibroblasts [5] of arthritis patients. Since the migration of T cells into tissue plays a role in both inflammatory diseases, inhibition of autotaxin may suppress this process and thus have a positive influence on the course of the disease.
1. Kanda, H., et al., Autotaxin, an ectoenzyme that produces lysophosphatidic acid, promotes the entry of lymphocytes into secondary lymphoid organs. Nat Immunol, 2008. 9(4): p. 415-23.
2. Nakasaki, T., et al., Involvement of the lysophosphatidic acid-generating enzyme autotaxin in lymphocyte-endothelial cell interactions. Am J Pathol, 2008. 173(5): p. 1566-76.
3. Wu, F., et al., Genome-wide gene expression differences in Crohn's disease and ulcerative colitis from endoscopic pinch biopsies: insights into distinctive pathogenesis. Inflamm Bowel Dis, 2007. 13(7): p. 807-21.
4. Nochi, H., et al., Stimulatory role of lysophosphatidic acid in cyclooxygenase-2 induction by synovial fluid of patients with rheumatoid arthritis in fibroblast-like synovial cells. J Immunol, 2008. 181(7): p. 5111-9.
5. Kehlen, A., et al., IL-1 beta- and IL-4-induced down-regulation of autotaxin mRNA and PC-1 in fibroblast-like synoviocytes of patients with rheumatoid arthritis (RA). Clin Exp Immunol, 2001. 123(1): p. 147-54.

Surprisingly, it has been found that the compounds according to the invention cause specific inhibition of the enzyme family of the nucleotides pyrophosphatases and phosphodiesterases, in particular autotaxin. The compounds according to the invention preferably exhibit an advantageous biological activity, which can easily be detected in the test described, for example, herein. In tests of this type, the compounds according to the invention preferably exhibit and cause an inhibiting effect, which is usually documented by IC₅₀ values in a suitable range, preferably in the micromolar range and more preferably in the nanomolar range.

In general, all solid and non-solid tumours can be treated with the compounds of the formula I, such as, for example, monocytic leukaemia, brain, urogenital, lymphatic system, stomach, laryngeal, ovarian and lung carcinoma, including lung adenocarcinoma and small-cell lung carcinoma. Further examples include prostate, pancreatic and breast carcinoma.

As discussed herein, effects of the compound according to the invention are relevant for various diseases. Accordingly, the compounds according to the invention are useful in the prophylaxis and/or treatment of diseases which are influenced by inhibition of one or more nucleotides pyrophosphatases and/or phosphodiesterases, in particular autotaxin.

The present invention therefore relates to compounds according to the invention as medicaments and/or medicament active ingredients in the treatment and/or prophylaxis of the said diseases and to the use of compounds according to the invention for the preparation of a pharmaceutical agent for the treatment and/or prophylaxis of the said diseases, and also to a method for the treatment of the said diseases comprising the administration of one or more compounds according to the invention to a patient in need of such administration.

It can be shown that the compounds according to the invention have an advantageous action in a xenotransplant tumour model.

The host or patient can belong to any mammalian species, for example a primate species, in particular humans; rodents, including mice, rats and hamsters; rabbits; horses, cattle, dogs, cats, etc. Animal models are of interest for experimental investigations, where they provide a model for the treatment of a human disease.

The sensitivity of a certain cell to treatment with the compounds according to the invention can be determined by testing in vitro. Typically, a culture of the cell is combined with a compound according to the invention at various concentrations for a time which is sufficient to enable the active agents to induce cell death or to inhibit cell migration or to block the cellular secretion of angiogenesis-promoting substances, usually between approximately one hour and one week. For testing in vitro, cultivated cells from a biopsy sample can be used. The viable cells remaining after the treatment are then counted.

The dose varies depending on the specific compound used, the specific disease, the patient status, etc. Typically, a therapeutic dose is sufficient to considerably reduce the undesired cell population in the target tissue, while the viability of the patient is maintained. The treatment is generally continued until a considerable reduction has occurred, for example at least about a 50% reduction in the cell burden, and can be continued until essentially no undesired cells can be detected in the body.

### PRIOR ART

Compounds which are capable of inhibiting autotaxin are described in Peng et al. Bioorganic & Medicinal Chemistry Letters (17, 2007, page 1634-1640). The compounds described therein are lipid analogues, which do not have any structural features in common with the compounds according to the invention.

### SUMMARY OF THE INVENTION

The invention relates to compounds of the formula I

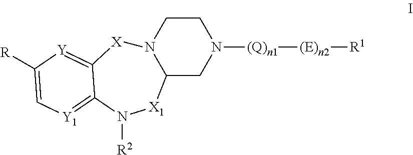

in which
R denotes Hal, Ar or Het¹,
R¹ denotes SO₂A, COOA, COOH, Cyc, Het, Ar, COHet, CONHHet, CONHAr, CHO, CONH₂, CONHA, CONA₂, (CH₂)ₙ₂OH, (CH₂)ₙ₂OA, OAr, NHAr, A, Hal, (CH₂)ₙ₂NH₂, (CH₂)ₙ₂NHA, (CH₂)ₙ₂NA₂ or NHCOA,
R² denotes H, (CH₂)ₙ₃NH₂, (CH₂)ₙ₃NHA, (CH₂)ₙ₃NA₂, (CH₂)ₙ₃OH, (CH₂)ₙ₃OA, (CH₂)ₙ₃Het², CH₂COHet², CH₂CONH₂, CH₂CONHA, CH₂CONA₂ or A,
X, X₁ each, independently of one another, denote CO, CH(OH), CH(OA), CH(NH₂), CH₂ or CF₂,
Y, Y₁ each, independently of one another, denote CH or N,
Q denotes C═O, COO, C═S, C═NH, CH(OH), CH(NH₂), SO, SO₂ or CF₂,
E denotes CO, CH(OH), CA(OH), CH(OA), CA(OA), CH(NH₂), Alk,

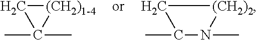

Alk denotes linear or branched alkylene having 1-8 C atoms, in which one or two CH₂ groups may be replaced by O and/or NH,
n1 denotes 0, 1 or 2,
n2 denotes 0, 1, 2, 3 or 4,
n3 denotes 1, 2, 3 or 4,
Ar denotes phenyl, naphthyl or biphenyl, each of which is unsubstituted or mono-, di- or trisubstituted by Hal, A, OH, OA, NH₂, NHA, NA₂, NO₂, CN, COOH, COOA, CONH₂, CONHA, CONA₂, NHCOA, NHSO₂A, SO₂NH₂ and/or SO₂A,
Het denotes a mono-, bi- or tricyclic saturated, unsaturated or aromatic heterocycle having 1 to 4 N, O and/or S atoms, which may be unsubstituted or mono-, di- or trisubstituted by Hal, Het², A, OH, OA, NH₂, NHA, NA₂, NO₂, CN, COOH, COOA, CONH₂, CONHA, CONA₂, NHCOA, NHSO₂A, SO₂NH₂, SO₂A, NHCONH₂, CHO, COA, ═S, ═NH, ═NA and/or ═O (carbonyl oxygen),
Het¹ denotes a mono-, bi- or tricyclic aromatic heterocycle having 1 to 4 N, O and/or S atoms, which may be unsubstituted or mono-, di- or trisubstituted by Hal, A, OH, OA, NH₂, NHA, NA₂, NO₂, CN, COOH, COOA, CONH₂, CONHA, CONA₂, NHCOA, NHSO₂A, SO₂NH₂, SO₂A, NHCONH₂, CHO and/or COA,
Het² denotes pyrrolidinyl, piperidinyl, thiazolidinyl, morpholinyl, oxazolidinyl, tetrahydroquinazolinyl, tetrahydropyranyl, piperazinyl, thiazolyl, furyl, thienyl, pyrrolyl, imidazolyl, pyrazolyl, oxazolyl, isoxazolyl, isothiazolyl, pyridyl, pyrimidinyl, triazolyl, tetrazolyl, oxadiazolyl or thiadiazolyl, each of which is unsubstituted or monosubstituted by A,
Cyc denotes cyclic alkyl having 3-7 C atoms,
A denotes unbranched or branched alkyl having 1-10 C atoms, in which 1-7H atoms may be replaced by F, Cl and/or Br, and/or in which one or two CH₂ groups may be replaced by O and/or NH,
   or
cyclic alkyl having 3-7 C atoms,
Hal denotes F, Cl, Br or I,
   and pharmaceutically usable salts and stereoisomers thereof, including mixtures thereof in all ratios,
   where the compounds “B1”-“B27”

| No. | Name and/or structure |
|---|---|
| “B1” | 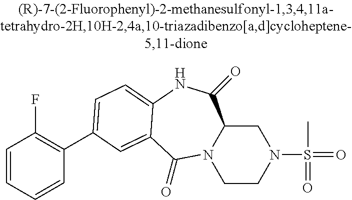 |
| “B2” | 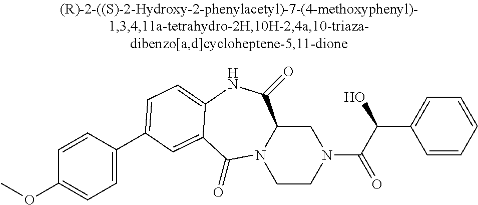 |
| “B3” | 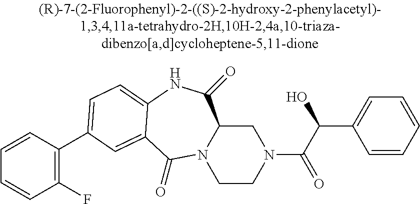 |
| “B4” | 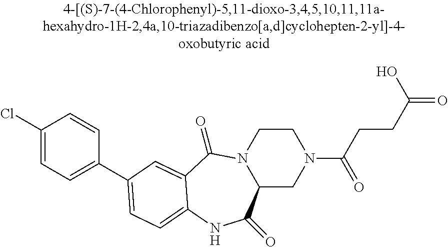 |
| “B5” | 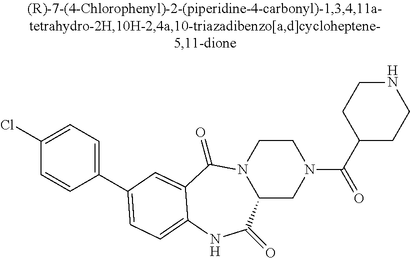 |
| “B6” | 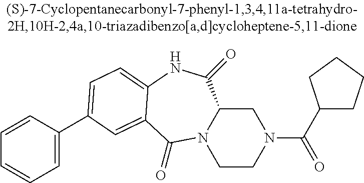 |
| “B7” | 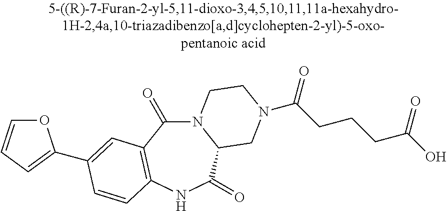 |
| “B8” | 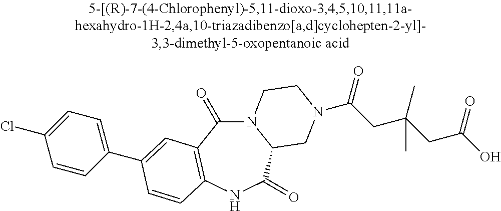 |
| “B9” | 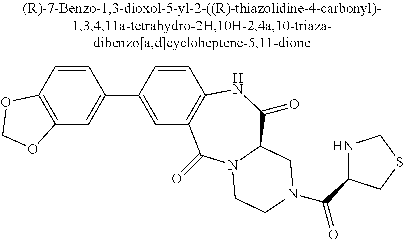 |
| “B10” | 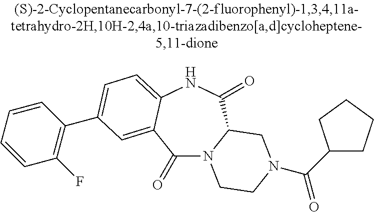 |
| “B11” | 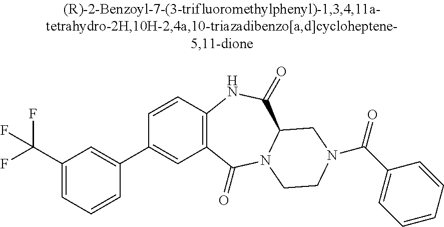 |
| “B12” | 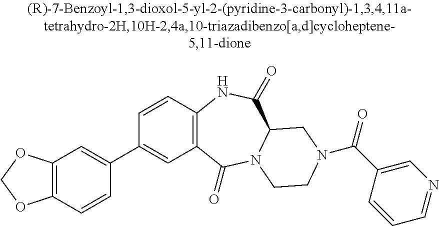 |
| “B13” | 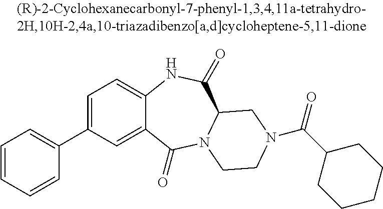 |
| “B14” | 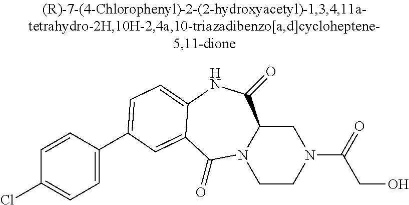 |
| “B15” | 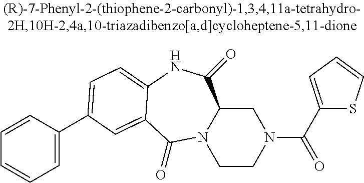 |
| “B16” | 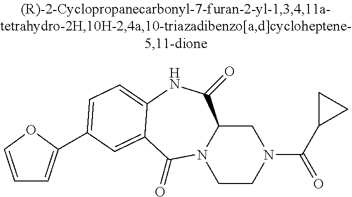 |
| “B17” | 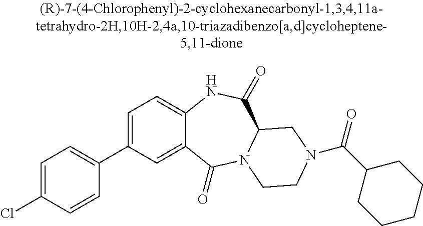 |
| “B18” | 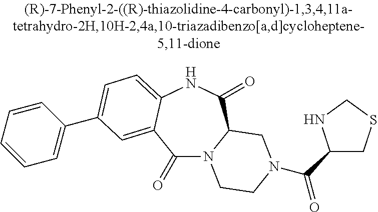 |
| “B19” | 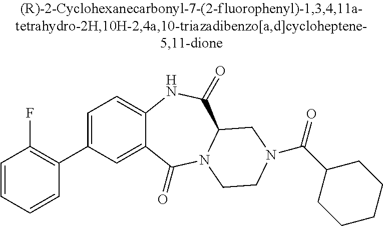 |
| “B20” | 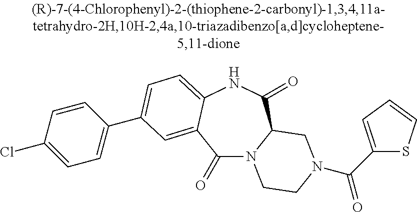 |
| “B21” | 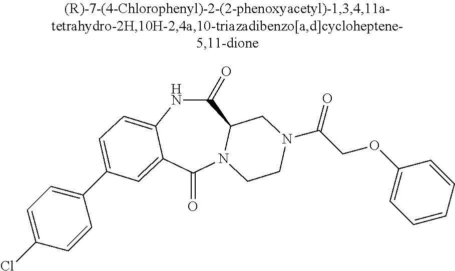 |
| “B22” | 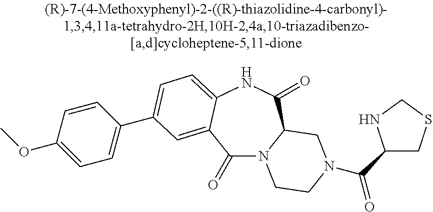 |
| “B23” | 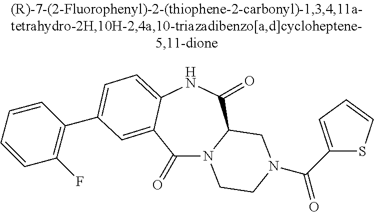 |
| “B24” | 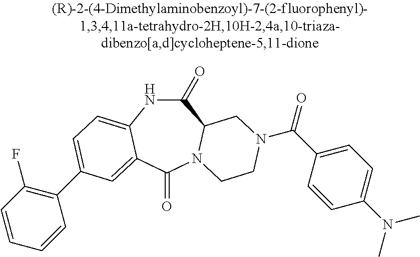 |
| “B25” | 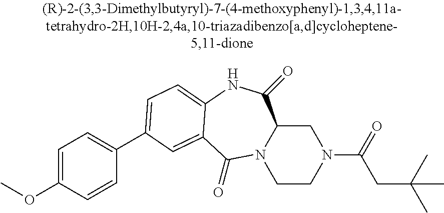 |
| “B26” | 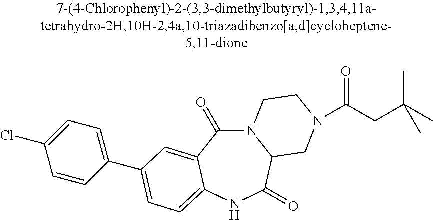 |
| “B27” | 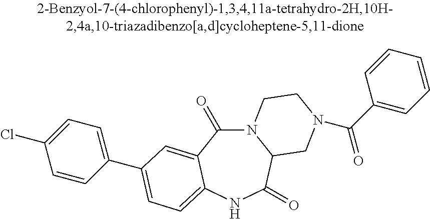 |

are excluded.

The present invention also relates to the use of the compounds of the formula I for the preparation of a medicament for the treatment of diseases in which the inhibition, regulation and/or modulation of the phosphodiesterase or lysophospholipase autotaxin plays a role.

The invention furthermore relates to medicaments comprising at least one compound according to claim 1 or a compound “B1”-“B27” and/or pharmaceutically usable salts and stereoisomers thereof, including mixtures thereof in all ratios, and optionally excipients and/or adjuvants.

The invention also relates to the compounds of the formula I and the compounds “B1”-“B27” and pharmaceutically usable salts and stereoisomers thereof, including mixtures thereof in all ratios, for the treatment and/or prophylaxis of tumours, tumour diseases and cancer diseases.

The invention furthermore relates to compounds selected from the group

| Compound | |
|---|---|
| No. | Name and/or structure |
| “10” | 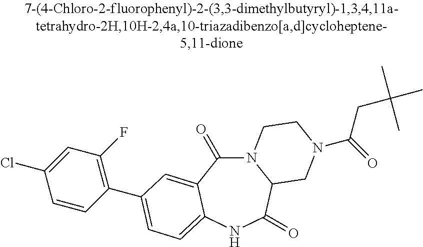 |
| “17” | 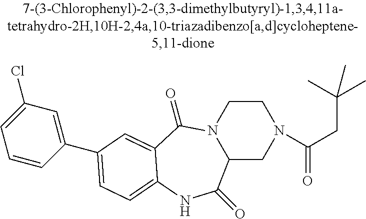 |
| “25” | 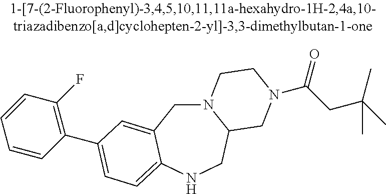 |
| “26” | 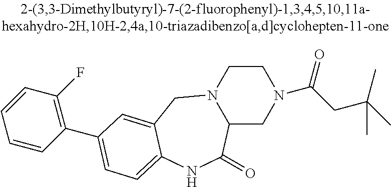 |
| “28” | 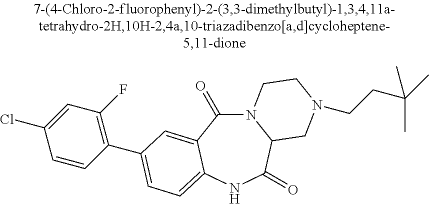 |
| “31” | 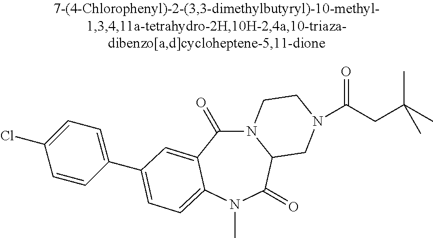 |
| “37” | 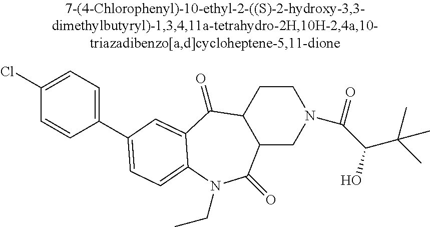 |
| “40” | Benzyl 7-(4-chlorophenyl)-5,11-dioxo-3,4,5,10,11,11a- |
| | hexahydro-1H-2,4a,10-triazadibenzo[a,d]cycloheptene-2- |
| | carboxylate |
| “41” | (R)-7-(4-Chloro-2-fluorophenyl)-2-(imidazole-1-carbonyl)- |
| | 1,3,4,11a-tetrahydro-2H,10H-2,4a,10-triaza- |
| | dibenzo[a,d]cycloheptene-5,11-dione |
| “48” | (R)-7-(4-Chloro-2-fluorophenyl)-2-[(S)-3,3-dimethyl-2- |
| | (piperidin-4-ylamino)butyryl]-1,3,4,11a-tetrahydro-2H,10H- |
| | 2,4a,10-triazadibenzo[a,d]cycloheptene-5,11-dione |
| “50” | (R)-7-(4-Chloro-2-fluorophenyl)-2-phenylmethanesulfonyl- |
| | 1,3,4,11a-tetrahydro-2H,10H-2,4a,10-triaza- |
| | dibenzo[a,d]cycloheptene-5,11-dione |
| “57” | 7-(4-Chlorophenyl)-2-((S)-2-hydroxy-3,3-dimethylbutyryl)- |
| | 1,3,4,5,10,11a-hexahydro-1H-2,4a,10-triazadibenzo[a,d]- |
| | cyclohepten-11-one |

and compounds “A1”-“A176” indicated in the examples

The compounds indicated below are described in the literature under their CAS No.

| Compound | | |
|---|---|---|
| No. | CAS No. | Name and/or structure |
| “B1” | 1009286- 73-0 | 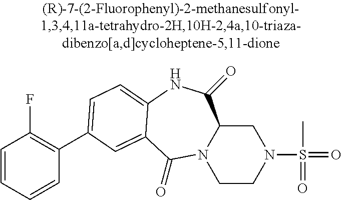 |
| “B2” | 1007918- 81-1 | 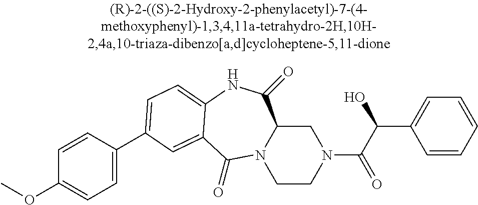 |
| “B3” | 1009763- 25-0 | 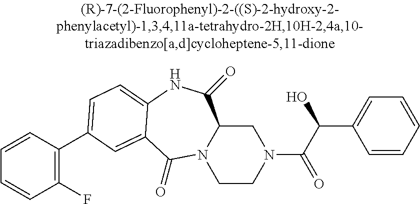 |
| “B4” | 1008455- 91-1 | 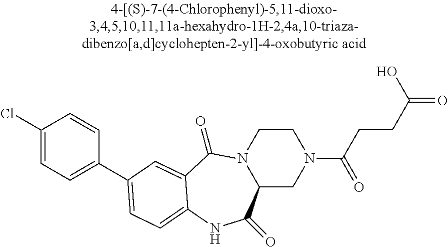 |
| “B5” | 1009416- 29-8 | 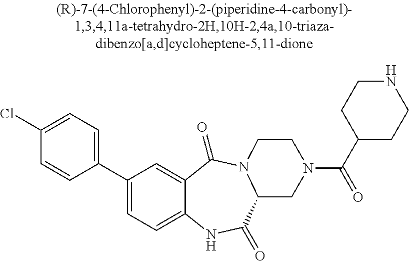 |
| “B6” | 1044747- 67-2 | 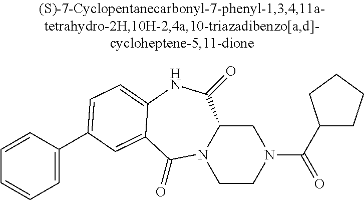 |
| “B7” | 1009702- 28-6 | 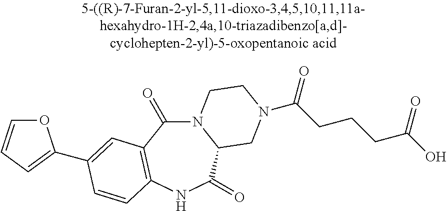 |
| “B8” | 1009702- 30-0 | 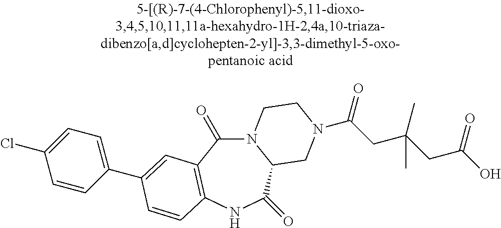 |
| “B9” | 1009180- 23-7 | 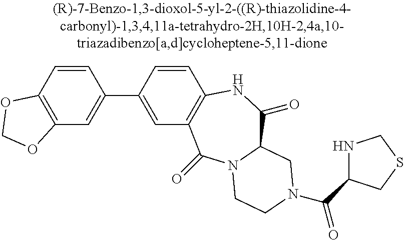 |
| “B10” | 1009713- 62-5 | 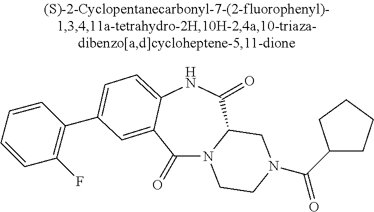 |
| “B11” | 1009317- 80-9 | 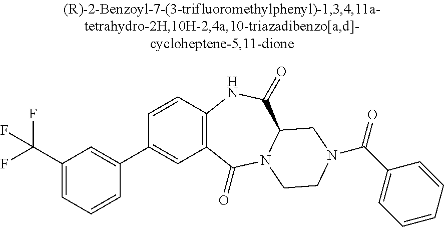 |
| “B12” | 1009787- 10-3 | 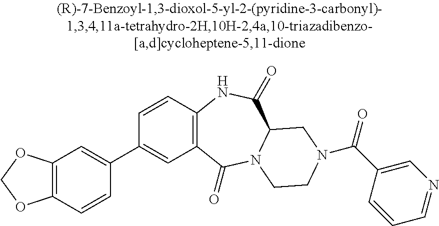 |
| “B13” | 1039033- 78-7 | 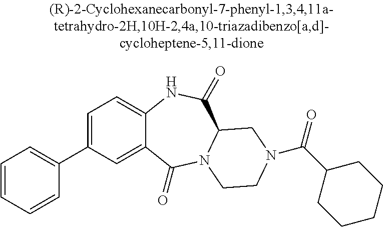 |
| “B14” | 1009745- 17-8 | 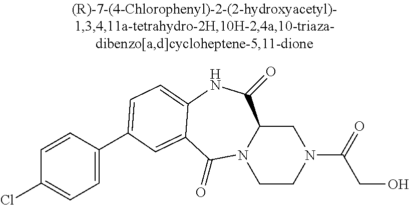 |
| “B15” | 1009286- 72-9 | 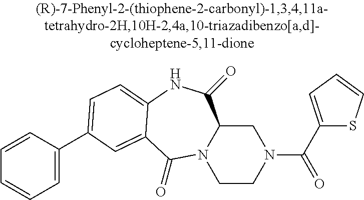 |
| “B16” | 1008483- 65-5 | 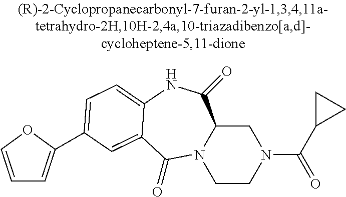 |
| “B17” | 1009751- 32-9 | 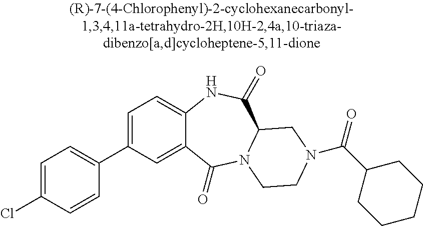 |
| “B18” | 1009597- 61-8 | 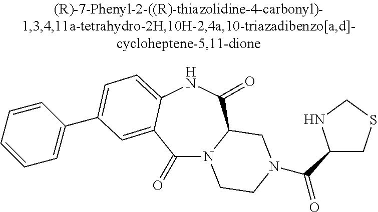 |
| “B19” | 1009179- 81-0 | 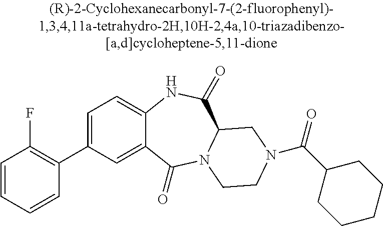 |
| “B20” | 1009751- 34-1 | 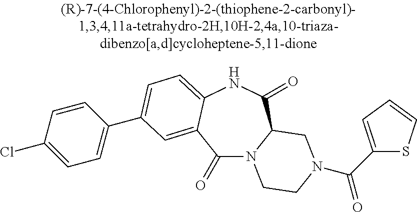 |
| “B21” | 1008488- 18-3 | 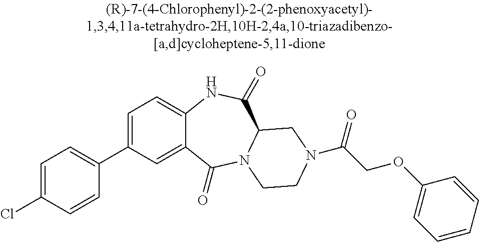 |
| “B22” | 1009416- 27-6 | 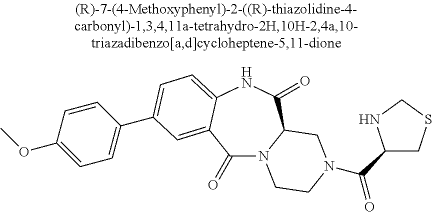 |
| “B23” | 1009286- 73-0 | 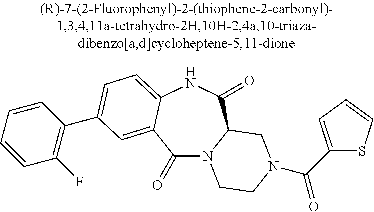 |
| “B24” | 1009745- 67-8 | 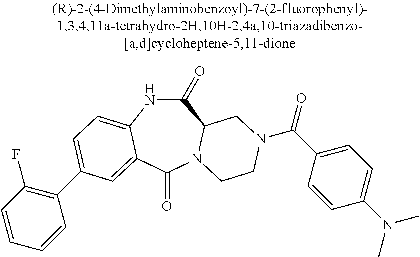 |
| “B25” | 1009335- 73-2 | 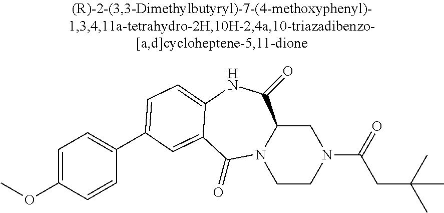 |
| “B26” | 1009709- 78-7 | 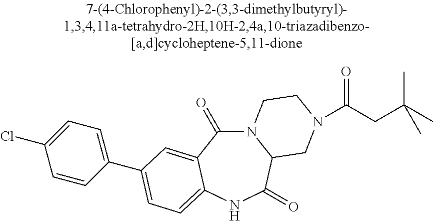 |
| “B27” | 1009757- 20-3 | 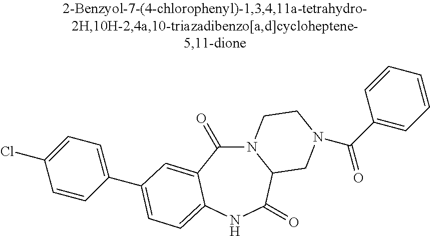 |

Compounds of the formula I also mean pharmaceutically usable derivatives thereof, optically active forms (stereoisomers), tautomers, polymorphs, enantiomers, racemates, diastereomers and the hydrates and solvates of these compounds. The term solvates of the compounds is taken to mean adductions of inert solvent molecules onto the compounds which form owing to their mutual attractive force. solvates are, for example, mono- or dihydrates or alcoholates.

Pharmaceutically usable derivatives are taken to mean, for example, the salts of the compounds according to the invention and also so-called pro-drug compounds.

Prodrug derivatives are taken to mean compounds of the formula I which have been modified by means of, for example, alkyl or acyl groups, sugars or oligopeptides and which are rapidly cleaved in the organism to form the effective compounds according to the invention.

These also include biodegradable polymer derivatives of the compounds according to the invention, as described, for example, in Int. J. Pharm. 115, 61-67 (1995).

The expression “effective amount” denotes the amount of a medicament or of a pharmaceutical active ingredient which causes in a tissue, system, animal or human a biological or medical response which is sought or desired, for example, by a researcher or physician.

In addition, the expression “therapeutically effective amount” denotes an amount which, compared with a corresponding subject who has not received this amount, has the following consequence: improved treatment, healing, prevention or elimination of a disease, syndrome, condition, complaint, disorder or side effects or also the reduction in the advance of a disease, complaint or disorder.

The expression “therapeutically effective amount” also encompasses the amounts which are effective for increasing normal physiological function.

The invention also relates to the use of mixtures of the compounds of the formula I, for example mixtures of two diastereomers, for example in the ratio 1:1, 1:2, 1:3, 1:4, 1:5, 1:10, 1:100 or 1:1000.

These are particularly preferably mixtures of stereoisomeric compounds.

A denotes alkyl and is preferably unbranched (linear) or branched, and has 1, 2, 3, 4, 5, 6, 7, 8, 9 or 10 C atoms. Alkyl preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethyl-propyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methyl-propyl, 1,1,2- or 1,2,2-trimethylpropyl, further preferably, for example, trifluoromethyl.

Alkyl very particularly preferably denotes alkyl having 1, 2, 3, 4, 5 or 6 C atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl.

One or two CH₂ groups in alkyl may also be replaced by O and/or NH.

Alkyl thus also denotes, for example, CH₂OCH₃ or NHCH₃.

Alkyl also denotes cycloalkyl.

Cycloalkyl preferably denotes cyclopropyl, cyclobutyl, cylopentyl, cyclohexyl or cycloheptyl.

Cyc denotes cyclopropyl, cyclobutyl, cylopentyl, cyclohexyl or cycloheptyl.

Alk denotes linear or branched alkylene having 1-8 C atoms, in which one or two CH₂ groups may be replaced by O and/or NH, preferably linear or branched methylene, ethylene, propylene, butylene, pentylene, hexylene, 2,2-dimethylpropylene, CH₂OCH₂, CH₂NHCH₂ or CH₂NH—.

R² preferably denotes H, (CH₂)ₙ₃NH₂, (CH₂)ₙ₃NHA, (CH₂)ₙ₃NA₂, (CH₂)ₙ₃OH, (CH₂)ₙ₃OA, (CH₂)ₙ₃Het², CH₂COHet², CH₂CONH₂, CH₂CONHA, CH₂CONA₂ or methyl.

R² very particularly preferably denotes H, (CH₂)₂NMe₂, (CH₂)₂OH, (CH₂)₂OMe, (CH₂)ₙ₃Het², CH₂COHet², CH₂CONH₂ or Me (methyl).

X, X₁ preferably denote, in each case independently of one another, CO or CH₂.

Y, Y₁ preferably denote CH.

Q preferably denotes CO, SO₂ or COO.

n1 preferably denotes 0 or 1.

n2 preferably denotes 0 or 1.

n3 preferably denotes 1 or 2.

Hal preferably denotes F, Cl or Br, but also I, particularly preferably Br or Cl.

Ar denotes, for example, phenyl, o-, m- or p-tolyl, o-, m- or p-ethylphenyl, o-, m- or p-propylphenyl, o-, m- or p-isopropylphenyl, o-, m- or p-tert-butyl-phenyl, o-, m- or p-hydroxyphenyl, o-, m- or p-nitrophenyl, o-, m- or p-aminophenyl, o-, m- or p-(N-methylamino)phenyl, o-, m- or p-(N-methyl-aminocarbonyl)phenyl, o-, m- or p-acetamidophenyl, o-, m- or p-methoxy-phenyl, o-, m- or p-ethoxyphenyl, o-, m- or p-ethoxycarbonylphenyl, o-, m- or p-(N,N-dimethylamino)phenyl, o-, m- or p-(N,N-dimethylaminocarbonyl)-phenyl, o-, m- or p-(N-ethylamino)phenyl, o-, m- or p-(N,N-diethylamino)-phenyl, o-, m- or p-fluorophenyl, o-, m- or p-bromophenyl, o-, m- or p-chlorophenyl, o-, m- or p-(methylsulfonamido)phenyl, o-, m- or p-(methyl-sulfonyl)phenyl, o-, m- or p-cyanophenyl, o-, m- or p-carboxyphenyl, o-, m- or p-methoxycarbonylphenyl, o-, m- or p-aminosulfonylphenyl, furthermore preferably 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromo-phenyl, 2,4- or 2,5-dinitrophenyl, 2,5- or 3,4-dimethoxyphenyl, 3-nitro-4-chlorophenyl, 3-amino-4-chloro-, 2-amino-3-chloro-, 2-amino-4-chloro-, 2-amino-5-chloro- or 2-amino-6-chlorophenyl, 2-nitro-4-N,N-dimethylamino- or 3-nitro-4-N,N-dimethylaminophenyl, 2,3-diaminophenyl, 2,3,4-, 2,3,5-, 2,3,6-, 2,4,6- or 3,4,5-trichlorophenyl, 2,4,6-trimethoxyphenyl, 2-hydroxy-3,5-dichlorophenyl, p-iodophenyl, 3,6-dichloro-4-aminophenyl, 4-fluoro-3-chlorophenyl, 2-fluoro-4-bromophenyl, 2,5-difluoro-4-bromophenyl, 3-bromo-6-methoxyphenyl, 3-chloro-6-methoxyphenyl, 3-chloro-4-acetamido-phenyl, 3-fluoro-4-methoxyphenyl, 3-amino-6-methylphenyl, 3-chloro-4-acetamidophenyl or 2,5-dimethyl-4-chlorophenyl.

Ar very particularly preferably denotes phenyl which is unsubstituted or mono-, di- or trisubstituted by Hal, A, OH, OA, NH₂, NHA and/or NA₂.

Irrespective of further substitutions, Het denotes, for example, 2- or 3-furyl, 2- or 3-thienyl, 1-, 2- or 3-pyrrolyl, 1-, 2,4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, indazolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, benzotriazolyl, further preferably 1,3-benzodioxol-5-yl, 1,4-benzodioxan-6-yl, 2,1,3-benzothiadiazol-4-, -5-yl or 2,1,3-benzoxadiazol-5-yl or dibenzo-furanyl.

The heterocyclic radicals may also be partially or fully hydrogenated. Irrespective of further substitutions, Het can thus also denote, for example, 2,3-dihydro-2-, -3-, -4- or -5-furyl, 2,5-dihydro-2-, -3-, -4- or 5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxanyl, 1,3-dioxan-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetra-hydro-1-, -2-, -3-, -4-, -5-, -6-, -7- or -8-quinolyl, 1,2,3,4-tetrahydro-1-,-2-,-3-, -4-, -5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or 6-yl, 2,3-(2-oxomethylenedioxy)phenyl or also 3,4-dihydro-2H-1,5-benzodioxepin-6- or -7-yl, furthermore preferably 2,3-dihydrobenzofuranyl, 2,3-dihydro-2-oxofuranyl, 3,4-dihydro-2-oxo-1H-quinazolinyl, 2,3-dihydrobenzoxazolyl, 2-oxo-2,3-dihydrobenzoxazolyl, 2,3-dihydrobenzimidazolyl, 1,3-dihydroindole, 2-oxo-1,3-dihydroindole or 2-oxo-2,3-dihydrobenzimidazolyl.

Het preferably denotes a mono-, bi- or tricyclic saturated, unsaturated or aromatic heterocycle having 1 to 4 N, O and/or S atoms, which may be unsubstituted or mono-, di- or trisubstituted by A, Het¹, OH, NH₂, NHA and/or NA₂.

Het very particularly preferably denotes pyrrolidinyl, piperidinyl, thiazolidinyl, morpholinyl, oxazolidinyl, tetrahydroquinazolinyl, piperazinyl, thiazolyl, furyl, thienyl, pyrrolyl, imidazolyl, pyrazolyl, oxazolyl, isoxazolyl, isothiazolyl, pyridyl, pyrimidinyl, triazolyl, tetrazolyl, oxadiazolyl, thiadiazolyl, imidazopyridinyl or benzotriazolyl, each of which is unsubstituted or mono-, di- or trisubstituted by A, Het¹, OH, NH₂, NHA and/or NA₂.

Irrespective of further substitutions, Het¹ denotes, for example, 2- or 3-furyl, 2- or 3-thienyl, 1-, 2- or 3-pyrrolyl, 1-, 2,4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or 5-yl, 1- or 5-tetrazolyl, 1,2,3-oxadiazol-4- or -5-yl, 1,2,4-oxadiazol-3- or -5-yl, 1,3,4-thia-diazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, 4- or 5-isoindolyl, indazolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, benzotriazolyl, further preferably 1,3-benzodioxol-5-yl, 1,4-benzodioxan-6-yl, 2,1,3-benzothiadiazol-4-, -5-yl or 2,1,3-benzoxadiazol-5-yl or dibenzofuranyl.

Het¹ preferably denotes a mono-, bi- or tricyclic aromatic heterocycle having 1 to 4 N, O and/or S atoms, which may be unsubstituted or mono-, di- or trisubstituted by A and/or Hal.

Het¹ very particularly preferably denotes furyl, thienyl, pyridyl, pyrrolyl, imidazolyl, pyrimidinyl or 1,3-benzodioxol-5-yl, where the radicals may be mono-, di- or trisubstituted by A and/or Hal.

Het² preferably denotes pyrrolidinyl, piperidinyl, thiazolidinyl, morpholinyl, oxazolidinyl, tetrahydroquinazolinyl, tetrahydropyranyl, piperazinyl, thiazolyl, furyl, thienyl, pyrrolyl, imidazolyl, pyrazolyl, oxazolyl, isoxazolyl, isothiazolyl, pyridyl, pyrimidinyl, triazolyl, tetrazolyl, oxadiazolyl or thiadiazolyl, each of which is unsubstituted or monosubstituted by A.

### Abbreviations:

DIPEA diisopropylethylamine
DAPECI N-3-dimethylaminopropyl-N′-ethylcarbodiimide
DCCI N,N′-dicyclohexylcarbodiimide
HOBt 1-hydroxybenzotriazole

Throughout the invention, all radicals which occur more than once, such as, for example, R, may be identical or different, i.e. are independent of one another.

The compounds of the formula I may have one or more chiral centres and can therefore occur in various stereoisomeric forms. The formula I encompasses all these forms.

Accordingly, the invention relates, in particular, to the use of the compounds of the formula I in which at least one of the said radicals has one of the preferred meanings indicated above.

Some preferred groups of compounds may be expressed by the following sub-formulae Ia to Ih, which conform to the formula I and in which the radi-cals not designated in greater detail have the meaning indicated for the formula I, but in which
in Ia R² denotes H, (CH₂)ₙ₃NH₂, (CH₂)ₙ₃NHA, (CH₂)ₙ₃NA₂, (CH₂)ₙ₃OH, (CH₂)ₙ₃OA, (CH₂)ₙ₃Het², CH₂COHet², CH₂CONH₂, CH₂CONHA, CH₂CONA₂ or methyl;
in Ib X, X₁ each, independently of one another, denote CO or CH₂;
in Ic Y, Y₁ denote CH;
in Id A denotes unbranched or branched alkyl having 1-10 C atoms, in which 1-7H atoms may be replaced by F and/or Cl,
   or
   cyclic alkyl having 3-7 C atoms;
in Ie Ar denotes phenyl which is unsubstituted or mono-, di- or trisubstituted by Hal, A, OH, OA, NH₂, NHA and/or NA₂;
in If Het denotes a mono-, bi- or tricyclic saturated, unsaturated or aro-matic heterocycle having 1 to 4 N, O and/or S atoms, which may be unsubstituted or mono-, di- or trisubstituted by A, Het², OH, NH₂, NHA and/or NA₂;
in Ig Het¹ denotes a mono-, bi- or tricyclic aromatic heterocycle having 1 to 4 N, O and/or S atoms, which may be unsubstituted or mono-, di- or trisubstituted by A and/or Hal;
in Ih R denotes Hal, Ar or Het¹,
   R¹ denotes SO₂A, COOA, COOH, Cyc, Het, Ar, COHet, CONHHet, CONHAr, CHO, CONH₂, CONHA, CONA₂, (CH₂)ₙ₂OH, (CH₂)ₙ₂OA, OAr, NHAr, (CH₂)ₙ₂NH₂, (CH₂)ₙ₂NHA, (CH₂)ₙ₂NA₂ or A,
   R² denotes H, (CH₂)ₙ₃NH₂, (CH₂)ₙ₃NHA, (CH₂)ₙ₃NA₂, (CH₂)ₙ₃OH, (CH₂)ₙ₃OA, (CH₂)ₙ₃Het², CH₂COHet², CH₂CONH₂, CH₂CONHA, CH₂CONA₂ or methyl,
   X, X₁ each, independently of one another, denote CO or CH₂,
   Y, Y₁ denote CH,
   Q denotes CO, SO₂ or COO,
   E denotes CO, CH(OH), CA(OH), CH(OA), CA(OA), CH(NH₂), Alk,

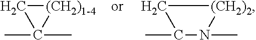

Alk denotes linear or branched alkylene having 1-8 C atoms, in which one or two CH₂ groups may be replaced by O and/or NH,
n1 denotes 0, 1 or 2,
n2 denotes 0, 1, 2, 3 or 4,
n3 denotes 1, 2, 3 or 4
Ar denotes phenyl which is unsubstituted or mono-, di- or trisubstituted by Hal, A, OH, OA, NH₂, NHA and/or NA₂,
Het denotes a mono-, bi- or tricyclic saturated, unsaturated or aro-matic heterocycle having 1 to 4 N, O and/or S atoms, which may be unsubstituted or mono-, di- or trisubstituted by A, Het², OH, NH₂, NHA and/or NA₂,
Het¹ denotes a mono-, bi- or tricyclic aromatic heterocycle having 1 to 4 N, O and/or S atoms, which may be unsubstituted or mono-, di- or trisubstituted by A and/or Hal,
Het² denotes pyrrolidinyl, piperidinyl, thiazolidinyl, morpholinyl, oxazolidinyl, tetrahydroquinazolinyl, tetrahydropyranyl, piperazinyl, thiazolyl, furyl, thienyl, pyrrolyl, imidazolyl, pyrazolyl, oxazolyl, isoxazolyl, isothiazolyl, pyridyl, pyrimidinyl, triazolyl, tetrazolyl, oxadiazolyl or thiadiazolyl, each of which is unsubstituted or monosubstituted by A,
Cyc denotes cyclic alkyl having 3-7 C atoms,
A denotes unbranched or branched alkyl having 1-10 C atoms, in which 1-7H atoms may be replaced by F and/or Cl, or
   cyclic alkyl having 3-7 C atoms,
Hal denotes F, Cl, Br or I;
   and pharmaceutically usable salts and stereoisomers thereof, including mixtures thereof in all ratios.

The compounds of the formula I and also the compounds according to the invention and also the starting materials for their preparation are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants known per se which are not mentioned here in greater detail.

Compounds of the formula I can preferably be obtained by reacting a compound of the formula II

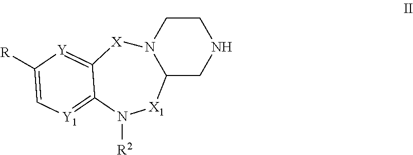

in which R, Y, Y₁, X, X₁ and R² have the meanings indicated in claim 1,
   with a compound of the formula III

L-(Q)ₙ₁-(E)ₙ₂-R¹ III
in which Q, E, R¹, n1 and n2 have the meanings indicated in claim 1 and
L denotes Cl, Br, I or a free or reactively functionally modified OH group.

In the compounds of the formula III, L preferably denotes Cl, Br, I or a free or a reactively modified OH group, such as, for example, an activated ester, an imidazolide or alkylsulfonyloxy having 1-6 C atoms (preferably methylsulfonyl-oxy or trifluoromethylsulfonyloxy) or arylsulfonyloxy having 6-10 C atoms (preferably phenyl- or p-tolylsulfonyloxy).

If L denotes OH in the compounds of the formula III, the reaction is particularly preferably carried out with addition of DAPECI and HOBT hydrate in DMF.

The reaction can also be carried out in the presence of an acid-binding agent, preferably an organic base, such as DIPEA, triethylamine, dimethylaniline, pyridine or quinoline. The addition of an alkali or alkaline-earth metal hydroxyide, carbonate or bicarbonate or another salt of a weak acid of the alkali or alkaline-earth metals, preferably of potassium, sodium, calcium or caesium, may also be favourable.

Depending on the conditions used, the reaction time is between a few minutes and 14 days, the reaction temperature is between about −30° and 140°, normally between −10° and 90°, in particular between about 0° and about 70°.

Examples of suitable inert solvents are hydrocarbons, such as hexane, petroleum ether, benzene, toluene or xylene; chlorinated hydrocarbons, such as trichloroethylene, 1,2-dichloroethane, carbon tetrachloride, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Particular preference is given to acetonitrile, dichloromethane and/or DMF.

Compounds of the formula I can furthermore preferably be obtained by reacting a compound of the formula IV

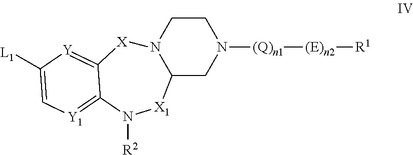

in which X, X₁, Y, Y₁, R¹, R², Q, E, n1 and n2 have the meanings indicated in claim 1 and L₁ denotes Br, CI or I, with a compound of the formula V

R—Y V
in which R has the meaning indicated in claim 1 and Y denotes a boronic acid or boronic acid ester radical

In the compounds of the formula V, Y preferably denotes

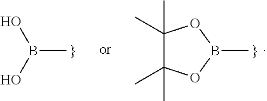

The reaction is carried out under standard conditions of a Suzuki coupling. Depending on the conditions used, the reaction time is between a few minutes and 14 days, the reaction temperature is between about −30° and 140°, normally between 0° and 100°, in particular between about 60° and about 90°.

The solvent is particularly preferably ethanol, toluene or dimethoxyethane.

The said individual compounds according to the invention can be used in their final non-salt form. On the other hand, the present invention also encompasses the use of these compounds in the form of their pharmaceutically acceptable salts, which can be derived from various organic and inorganic acids and bases by procedures known in the art. Pharmaceutically acceptable salt forms of the compounds are for the most part prepared by conventional methods. If the compound contains a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline-earth metal hydroxides, such as barium hydroxide and calcium hydroxide; alkali metal alkoxides, for example potassium ethoxide and sodium propoxide; and various organic bases, such as piperidine, diethanolamine and N-methyl-glutamine. The aluminium salts of the compounds are likewise included. In the case of certain compounds, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as ethanesulfonate, toluenesulfonate and benzenesulfonate, and other organic acids and corresponding salts thereof, such as acetate, trifluoroacetate, tartrate, maleate, succinate, citrate, benzoate, salicylate, ascorbate and the like. Accordingly, pharmaceutically acceptable acid-addition salts of the compounds include the following: acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, phthalate, but this does not represent a restriction.

Furthermore, the base salts of the compounds include aluminium, ammonium, calcium, copper, iron(III), iron(II), lithium, magnesium, manganese(III), manganese(II), potassium, sodium and zinc salts, but this is not intended to represent a restriction. Of the above-mentioned salts, preference is given to ammonium; the alkali metal salts sodium and potassium, and the alkaline-earth metal salts calcium and magnesium. Salts of the compounds which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzylethylenediamine (benzathine), dicyclohexylamine, diethanolamine, diethylamine, 2-diethyl-aminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tri-propylamine and tris(hydroxymethyl)methylamine(tromethamine), but this is not intended to represent a restriction.

Compounds of the present invention which contain basic nitrogen-containing groups can be quaternised using agents such as (C₁-C₄)alkyl halides, for example methyl, ethyl, isopropyl and tert-butyl chloride, bromide and iodide; di(C₁-C₄)alkyl sulfates, for example dimethyl, diethyl and diamyl sulfate; (C₁₀-C₁₈)alkyl halides, for example decyl, dodecyl, lauryl, myristyl and stearyl chloride, bromide and iodide; and aryl(C₁-C₄)alkyl halides, for example benzyl chloride and phenethyl bromide. Both water- and oil-soluble compounds according to the invention can be prepared using such salts.

The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, meglumine, nitrate, oleate, phosphonate, pivalate, sodium phosphate, stearate, sulfate, sulfosalicylate, tartrate, thiomalate, tosylate and tromethamine, but this is not intended to represent a restriction.

The acid-addition salts of basic compounds are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts otherwise correspond to the respective free base forms thereof.

As mentioned, the pharmaceutically acceptable base-addition salts of the compounds are formed with metals or amines, such as alkali metals and alkaline-earth metals or organic amines. Preferred metals are sodium, potassium, magnesium and calcium. Preferred organic amines are N,N′-dibenzylethylenediamine, chloroprocaine, choline, diethanolamine, ethylenediamine, N-methyl-D-glucamine and procaine.

The base-addition salts of acidic compounds according to the invention are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.

If a compound according to the invention contains more than one group which is capable of forming pharmaceutically acceptable salts of this type, the invention also encompasses multiple salts. Typical multiple salt forms include, for example, bitartrate, diacetate, difumarate, dimeglumine, diphosphate, disodium and trihydrochloride, but this is not intended to represent a restriction.

With regard to that stated above, it can be seen that the expression “pharmaceutically acceptable salt” in the present connection is taken to mean an active ingredient which comprises a compound in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

The invention furthermore relates to medicaments comprising at least one compound according to the invention and/or pharmaceutically usable salts and stereoisomers thereof, including mixtures thereof in all ratios, and optionally excipients and/or adjuvants.

Pharmaceutical formulations can be administered in the form of dosage units which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tabletting machine, giving lumps of non-uniform shape, which are broken up to form granules. The granules can be lubricated by addition of stearic acid, a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compound. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds and salts, solvates and physiologically functional derivatives thereof can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds and the salts, solvates and physiologically functional derivatives thereof can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurized dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary. Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound depends on a number of factors, including, for example, the age and weight of the animal, the precise condition that requires treatment, and its severity, the nature of the formulation and the method of administration, and is ultimately determined by the treating doctor or vet. However, an effective amount of a compound according to the invention for the treatment of neoplastic growth, for example colon or breast carcinoma, is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as a single dose per day or more usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention per se. It can be assumed that similar doses are suitable for the treatment of other conditions mentioned above.

The invention furthermore relates to medicaments comprising at least one compound according to the invention and/or pharmaceutically usable derivatives, solvates and stereoisomers thereof, including mixtures thereof in all ratios, and at least one further medicament active ingredient.

The invention also relates to a set (kit) consisting of separate packs of
(a) an effective amount of a compound according to the invention and/or pharmaceutically usable derivatives, solvates and stereoisomers thereof, including mixtures thereof in all ratios, and
(b) an effective amount of a further medicament active ingredient.

The set comprises suitable containers, such as boxes, individual bottles, bags or ampoules. The set may, for example, comprise separate ampoules, each containing an effective amount of a compound according to the invention and/or pharmaceutically usable derivatives, solvates and stereoisomers thereof, including mixtures thereof in all ratios, and an effective amount of a further medicament active ingredient in dissolved or lyophilised form.

The medicaments from Table 1 are preferably, but not exclusively, combined with the compounds according to the invention.

TABLE 1

| | | |
|---|---|---|
| Alkylating agents | Cyclophosphamide | Lomustine |
| | Busulfan | Procarbazine |
| | Ifosfamide | Altretamine |
| | Melphalan | Estramustine phosphate |
| | Hexamethylmelamine | Mechloroethamine |
| | Thiotepa | Streptozocin |
| | chloroambucil | Temozolomide |
| | Dacarbazine | Semustine |
| | Carmustine | |
| Platinum agents | Cisplatin | Carboplatin |
| | Oxaliplatin | ZD-0473 (AnorMED) |
| | Spiroplatin | Lobaplatin (Aetema) |
| | Carboxyphthalatoplatinum | Satraplatin (Johnson Matthey) |
| | Tetraplatin | BBR-3464 (Hoffrnann-La |
| | Ormiplatin | Roche) |
| | Iproplatin | SM-11355 (Sumitomo) |
| | | AP-5280 (Access) |
| Antimetabolites | Azacytidine | Tomudex |
| | Gemcitabine | Trimetrexate |
| | Capecitabine | Deoxycoformycin |
| | 5-fluorouracil | Fludarabine |
| | Floxuridine | Pentostatin |
| | 2-chlorodesoxyadenosine | Raltitrexed |
| | 6-Mercaptopurine | Hydroxyurea |
| | 6-Thioguanine | Decitabine (SuperGen) |
| | Cytarabine | Clofarabine (Bioenvision) |
| | 2-fluorodesoxycytidine | Irofulven (MGI Pharrna) |
| | Methotrexate | DMDC (Hoffmann-La Roche) |
| | Idatrexate | Ethynylcytidine (Taiho ) |
| Topoisomerase inhibitors | Amsacrine | Rubitecan (SuperGen) |
| | Epirubicin | Exatecan mesylate (Daiichi) |
| | Etoposide | Quinamed (ChemGenex) |
| | Teniposide or mitoxantrone | Gimatecan (Sigma-Tau) |
| | Irinotecan (CPT-11) | Diflomotecan (Beaufour- |
| | 7-Ethyl-10- | Ipsen) |
| | hydroxycamptothecin | TAS-103 (Taiho) |
| | Topotecan | Elsamitrucin (Spectrum) |
| | Dexrazoxanet (TopoTarget) | J-107088 (Merck & Co) |
| | Pixantrone (Novuspharrna) | BNP-1350 (BioNumerik) |
| | Rebeccamycin analogue | CKD-602 (Chong Kun Dang) |
| | (Exelixis) | KW-2170 (Kyowa Hakko) |
| | BBR-3576 (Novuspharrna) | |
| Antitumour antibiotics | Dactinomycin (Actinomycin | Amonafide |
| | D) | Azonafide |
| | Doxorubicin (Adriamycin) | Anthrapyrazole |
| | Deoxyrubicin | Oxantrazole |
| | Valrubicin | Losoxantrone |
| | Daunorubicin (Daunomycin) | Bleomycin sulfate (Blenoxan) |
| | Epirubicin | Bleomycinic acid |
| | Therarubicin | Bleomycin A |
| | Idarubicin | Bleomycin B |
| | Rubidazone | Mitomycin C |
| | Plicamycinp | MEN-10755 (Menarini) |
| | Porfiromycin | GPX-100 (Gem |
| | Cyanomorpholinodoxorubici | Pharmaceuticals) |
| | Mitoxantrone (Novantrone) | |
| Antimitotic agents | Paclitaxel | SB 408075 (GlaxoSmithKline) |
| | Docetaxel | E7010 (Abbott) |
| | Colchicine | PG-TXL (Cell Therapeutics) |
| | Vinblastine | IDN 5109 (Bayer) |
| | Vincristine | A 105972 (Abbott) |
| | Vinorelbine | A 204197 (Abbott) |
| | Vindesine | LU 223651 (BASF) |
| | Dolastatin 10 (NCI) | D 24851 (ASTA Medica) |
| | Rhizoxin (Fujisawa) | ER-86526 (Eisai) |
| | Mivobulin (Warner-Lambert) | Combretastatin A4 (BMS) |
| | Cemadotin (BASF) | Isohomohalichondrin-B |
| | RPR 109881A (Aventis) | (PharmaMar) |
| | TXD 258 (Aventis) | ZD 6126 (AstraZeneca) |
| | Epothilone B (Novartis) | PEG-Paclitaxel (Enzon) |
| | T 900607 (Tularik) | AZ10992 (Asahi) |
| | T 138067 (Tularik) | !DN-5109 (Indena) |
| | Cryptophycin 52 (Eli Lilly) | AVLB (Prescient |
| | Vinflunine (Fabre) | NeuroPharma) |
| | Auristatin PE (Teikoku | Azaepothilon B (BMS) |
| | hormone) | BNP-7787 (BioNumerik) |
| | BMS 247550 (BMS) | CA-4-prodrug (OXiGENE) |
| | BMS 184476 (BMS) | Dolastatin-10 (NrH) |
| | BMS 188797 (BMS) | CA-4 (OXiGENE) |
| | Taxoprexin (Protarga) | |
| Aromatase inhibitors | Aminoglutethimide | Exemestan |
| | Letrozole | Atamestan (BioMedicines) |
| | Anastrazole | YM-511 (Yamanouchi) |
| | Formestan | |
| Thymidylate synthase | Pemetrexed (Eli Lilly) | Nolatrexed (Eximias) |
| inhibitors | ZD-9331 (BTG) | CoFactor ™ (BioKeys) |
| DNA antagonists | Trabectedin (PharmaMar) | Mafosfamide (Baxter |
| | Glufosfamide (Baxter | International) |
| | International) | Apaziquone (Spectrum |
| | Albumin + 32P (Isotope | Pharmaceuticals) |
| | Solutions) | O6-benzylguanine (Paligent) |
| | Thymectacin (NewBiotics) | |
| | Edotreotid (Novartis) | |
| Farnesyl transferase | Arglabin (NuOncology Labs) | Tipifarnib (Johnson & |
| inhibitors | Ionafarnib (Schering-Plough | Johnson) |
| | BAY-43-9006 (Bayer) | Perillyl alcohol (DOR |
| | | BioPharma) |
| Pump inhibitors | CBT-1 (CBA Pharma) | Zosuquidar trihydrochloride |
| | Tariquidar (Xenova) | (Eli Lilly) |
| | MS-209 (Schering AG) | Biricodar dicitrate (Vertex) |
| Histone acetyl | Tacedinaline (Pfizer) | Pivaloyloxymethyl butyrate |
| transferase inhibitors | SAHA (Aton Pharma) | (Titan) |
| | MS-275 (Schering AG) | Depsipeptide (Fujisawa) |
| Metalloproteinase | Neovastat (Aeterna Labo- | CMT-3 (CollaGenex) |
| inhibitors | ratories) | BMS-275291 (Celltech) |
| Ribonucleoside | Marimastat (British Biotech) | Tezacitabine (Aventis) |
| reductase inhibitors | Gallium maltolate (Titan) | Didox (Molecules for Health) |
| | Triapin (Vion) | |
| TNF-alpha | Virulizin (Lorus | Revimid (Celgene) |
| agonists/antagonists | Therapeutics) | |
| | CDC-394 (Celgene) | |
| Endothelin-A | Atrasentan (Abbot) | YM-598 (Yamanouchi) |
| receptor antagonists | ZD-4054 (AstraZeneca) | |
| Retinoic acid | Fenretinide (Johnson & | Alitretinoin (Ligand) |
| receptor agonists | Johnson) | |
| | LGD-1550 (Ligand) | |
| Immunomodulators | Interferon | Dexosome therapy (Anosys) |
| | Oncophage (Antigenics) | Pentrix (Australian Cancer |
| | GMK (Progenics) | Technology) |
| | Adenocarcinoma vaccine | JSF-154 (Tragen) |
| | (Biomira) | Cancer vaccine (Intercell) |
| | CTP-37 (AVI BioPharma) | Norelin (Biostar) |
| | JRX-2 (Immuno-Rx) | BLP-25 (Biomira) |
| | PEP-005 (Peplin Biotech) | MGV (Progenics) |
| | Synchrovax vaccines (CTL | !3-Alethin (Dovetail) |
| | Immuno) | CLL-Thera (Vasogen) |
| | Melanoma vaccine (CTL | |
| | Immuno) | |
| | p21-RAS vaccine | |
| | (GemVax) | |
| Hormonal and | Oestrogens | Prednisone |
| antihormonal agents | Conjugated oestrogens | Methylprednisolone |
| | Ethynyloestradiol | Prednisolone |
| | chlorotrianisene | Aminoglutethimide |
| | Idenestrol | Leuprolide |
| | Hydroxyprogesterone capro | Goserelin |
| | Medroxyprogesterone | Leuporelin |
| | Testosterone | Bicalutamide |
| | Testosterone propionate | Flutamide |
| | Fluoxymesterone | Octreotide |
| | Methyltestosterone | Nilutamide |
| | Diethylstilbestrol | Mitotan |
| | Megestrol | P-04 (Novogen) |
| | Tamoxifen | 2-Methoxyoestradiol |
| | Toremofin | (EntreMed) |
| | Dexamethasone | Arzoxifen (Eli Lilly) |
| Photodynamic agents | Talaporfin (Light Sciences) | Pd-bacteriopheophorbide |
| | Theralux | (Yeda) |
| | (Theratechnologies) | Lutetium texaphyrin |
| | Motexafin gadolinium | (Pharmacyclics) |
| | (Pharmacyclics) | Hypericin |
| Tyrosine kinase inhibitors | Imatinib (Novartis) | Kahalide F (PharmaMar) |
| | Leflunomide | CEP-701 (Cephalon) |
| | (Sugen/Pharmacia) | CEP-751 (Cephalon) |
| | ZDI839 (AstraZeneca) | MLN518 (Millenium) |
| | Erlotinib (Oncogene | PKC412 (Novartis) |
| | Science) | Phenoxodiol O |
| | Canertjnib (Pfizer) | Trastuzumab (Genentech) |
| | Squalamine (Genaera) | C225 (ImClone) |
| | SU5416 (Pharmacia) | rhu-Mab (Genentech) |
| | SU6668 (Pharmacia) | MDX-H210 (Medarex) |
| | ZD4190 (AstraZeneca) | 2C4 (Genentech) |
| | ZD6474 (AstraZeneca) | MDX-447 (Medarex) |
| | Vatalanib (Novartis) | ABX-EGF (Abgenix) |
| | PKI166 (Novartis) | IMC-1C11 (ImClone) |
| | GW2016 (GlaxoSmithKline) | |
| | EKB-509 (Wyeth) | |
| | EKB-569 (Wyeth) | |
| Various agents | SR-27897 (CCK-A inhibitor, | BCX-1777 (PNP inhibitor, |
| | Sanofi-Synthelabo) | BioCryst) |
| | Tocladesine (cyclic AMP | Ranpirnase (ribonuclease |
| | agonist, Ribapharm) | stimulant, Alfacell) |
| | Alvocidib (CDK inhibitor, | Galarubicin (RNA synthesis |
| | Aventis) | inhibitor, Dong-A) |
| | CV-247 (COX-2 inhibitor, | Tirapazamine |
| | Ivy Medical) | (reducing agent, SRI |
| | P54 (COX-2 inhibitor, | International) |
| | Phytopharm) | N-Acetylcysteine |
| | CapCell ™ (CYP450 | (reducing agent, Zambon) |
| | stimulant, Bavarian Nordic) | R-Flurbiprofen (NF-kappaB |
| | GCS-IOO (gal3 antagonist, | inhibitor, Encore) |
| | GlycoGenesys) | 3CPA (NF-kappaB inhibitor, |
| | G17DT immunogen (gastrin | Active Biotech) |
| | inhibitor, Aphton) | Seocalcitol (vitamin D receptor |
| | Efaproxiral (oxygenator, | agonist, Leo) |
| | Allos Therapeutics) | 131-I-TM-601 (DNA |
| | PI-88 (heparanase inhibitor, | antagonist, TransMolecular) |
| | Progen) | Eflornithin (ODC inhibitor, |
| | Tesmilifen (histamine | ILEX Oncology) |
| | antagonist, YM | Minodronic acid (osteoclast |
| | BioSciences) | inhibitor, Yamanouchi) |
| | Histamine (histamine H2 | Indisulam (p53 stimulant, |
| | receptor agonist, Maxim) | Eisai) |
| | Tiazofurin (IMPDH inhibitor, | Aplidin (PPT inhibitor, |
| | Ribapharm) | PharmaMar) |
| | Cilengitide (integrin | Rituximab (CD20 antibody, |
| | antagonist, Merck KGaA) | Genentech) |
| | SR-31747 (IL-1 antagonist, | Gemtuzumab (CD33 antibody, |
| | Sanofi-Synthelabo) | Wyeth Ayerst) |
| | CCI-779 (mTOR kinase | PG2 (haematopoiesis |
| | inhibitor, Wyeth) | promoter, Pharmagenesis) |
| | Exisulind (PDE-V inhibitor, | Immunol ™ (triclosan |
| | Cell Pathways) | mouthwash, Endo) |
| | CP-461 (PDE-V inhibitor, | Triacetyluridine (uridine |
| | Cell Pathways) | prodrug, Wellstat) |
| | AG-2037 (GART inhibitor, | SN-4071 (sarcoma agent, |
| | Pfizer) | Signature BioScience) |
| | WX-UK1 | TransMID-107 ™ |
| | (plasminogen activator- | (immunotoxin, KS Biomedix) |
| | inhibitor, Wilex) | PCK-3145 (apoptosis |
| | PBI-1402 (PMN stimulant, | promoter, Procyon) |
| | ProMetic LifeSciences) | Doranidazole (apoptosis |
| | Bortezomib (proteasome | promoter, Pola) |
| | inhibitor, Millennium) | CHS-828 (cytotoxic |
| | SRL-172 (T-cell stimulant, | agent, Leo) |
| | SR Pharma) | trans-Retinoic acid |
| | TLK-286 (glutathione-S | (differentiator, NIH) |
| | transferase inhibitor, Telik) | MX6 (apoptosis promoter, |
| | PT-100 (growth factor | MAXIA) |
| | agonist, Point Therapeutics) | Apomine (apoptosis promoter, |
| | Midostaurin (PKC inhibitor, | ILEX Oncology) |
| | Novartis) | Urocidin (apoptosis promoter, |
| | Bryostatin-1 (PKC stimulant | Bioniche) |
| | GPC Biotech) | Ro-31-7453 (apoptosis |
| | CDA-II (apoptosis promoter, | promoter, La Roche) |
| | Everlife) | Brostallicin (apoptosis |
| | SDX-101 (apoptosis | promoter, Pharmacia) |
| | promoter, Salmedix) | |
| | Ceflatonin (apoptosis | |
| | promoter, ChemGenex) | |

The compounds of the formula I are preferably combined with the with known anti-cancer agents:

These known anti-cancer agents include the following: oestrogen receptor modulators, androgen receptor modulators, retinoid receptor modulators, cytotoxic agents, antiproliferative agents, prenylprotein transferase inhibitors, HMG-CoA reductase inhibitors, HIV protease inhibitors, reverse transcriptase inhibitors and other angiogenesis inhibitors. The present compounds are particularly suitable for administration at the same time as radiotherapy. The synergistic effects of inhibition of VEGF in combination with radiotherapy have been described in the art (see WO 00/61186).

“Oestrogen receptor modulators” refers to compounds which interfere with or inhibit the binding of oestrogen to the receptor, regardless of mechanism. Examples of oestrogen receptor modulators include, but are not limited to, tamoxifen, raloxifene, idoxifene, LY353381, LY 117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]phenyl 2,2-dimethylpropanoate, 4,4′-dihydroxybenzophenone-2,4-dinitrophenylhydrazone and SH646.

“Androgen receptor modulators” refers to compounds which interfere with or inhibit the binding of androgens to the receptor, regardless of mechanism. Examples of androgen receptor modulators include finasteride and other 5α-reductase inhibitors, nilutamide, flutamide, bicalutamide, liarozole and abiraterone acetate.

“Retinoid receptor modulators” refers to compounds which interfere with or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl)retinamide and N-4-carboxyphenyl-retinamide.

“Cytotoxic agents” refers to compounds which result in cell death primarily through direct action on the cellular function or inhibit or interfere with cell myosis, including alkylating agents, tumour necrosis factors, intercalators, microtubulin inhibitors and topoisomerase inhibitors.

Examples of cytotoxic agents include, but are not limited to, tirapazimine, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosylate, trofosfamide, nimustine, dibrospidium chloride, pumitepa, lobaplatin, satraplatin, profiromycin, cisplatin, irofulven, dexifosfamide, cis-aminedichloro(2-methylpyridine)platinum, benzylguanine, glufosfamide, GPX100, (trans,trans,trans)bis-mu-(hexane-1,6-diamine)mu-[diamine-platinum(II)]bis[diamine(chloro)platinum(II)] tetrachloride, diarisidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplastone, 3′-deamino-3′-morpholino-13-deoxo-10-hydroxycaminomycin, annamycin, galarubicin, elinafide, MEN10755 and 4-demethoxy-3-deamino-3-aziridinyl-4-methylsulfonyldaunorubicin (see WO 00/50032).

Examples of microtubulin inhibitors include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzenesulfonamide, anhydrovinblastine, N,N-dimethyl-L-valyl-L-valyl-N-methyl-L-valyl-L-prolyl-L-proline-t-butylamide, TDX258 and BMS188797.

Some examples of topoisomerase inhibitors are topotecan, hycaptamine, irinotecan, rubitecan, 6-ethoxypropionyl-3′,4′-O-exobenzylidenechartreusin, 9-methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methyl-1H,12H-benzo[de]pyrano[3′,4′:b,7]indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxyetoposide, GL331, N-[2-(dimethylamino)-ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methyl-amino]ethyl]-5-[4-hydroxy-3,5-dimethoxyphenyl]-5,5a,6,8,8a,9-hexohydrofuro(3′,′:6,7)naphtho(2,3-d)-1,3-dioxol-6-one, 2,3-(methylenedioxy)-5-methyl-7-hydroxy-8-methoxybenzo[c]phenanthridinium, 6,9-bis[(2-amino-ethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one and dimesna.

“Antiproliferative agents” include antisense RNA and DNA oligonucleotides such as G3139, ODN698, RVASKRAS, GEM231 and INX3001 and anti-metabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-mannoheptopyrano-syl]adenine, aplidine, ecteinascidin, troxacitabine, 4-[2-amino-4-oxo-4,6,7,8-tetrahydro-3H-pyrimidino[5,4-b]-1,4-thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-ylacetic acid ester, swainsonine, lometrexol, dexrazoxane, methioninase, 2′-cyano-2′-deoxy-N4-palmitoyl-1-B-D-arabino-furanosyl cytosine and 3-aminopyridine-2-carboxaldehyde thiosemicarbazone. “Antiproliferative agents” also include monoclonal antibodies to growth factors other than those listed under “angiogenesis inhibitors”, such as trastuzumab, and tumour suppressor genes, such as p53, which can be delivered via recombinant virus-mediated gene transfer (see U.S. Pat. No. 6,069,134, for example).

Particular preference is given to the use of the compounds of the formula I for the treatment and prophylaxis of tumours and/or tumour diseases and for the prophylaxis of cancer diseases.

The tumour is preferably selected from the group of tumours of the squamous epithelium, of the bladder, of the stomach, of the kidneys, of head and neck, of the oesophagus, of the cervix, of the thyroid, of the intestine, of the liver, of the brain, of the prostate, of the urogenital tract, of the lymphatic system, of the stomach, of the larynx and/or of the lung.

The tumour is furthermore preferably selected from the group lung adenocarcinoma, small-cell lung carcinomas, pancreatic cancer, ovarian carcinoma, glioblastomas, colon carcinoma and breast carcinoma.

Preference is furthermore given to the use for the treatment of a tumour of the blood and immune system, preferably for the treatment of a tumour selected from the group of acute myeloid leukaemia, chronic myeloid leukaemia, acute lymphatic leukaemia and/or chronic lymphatic leukaemia.

In another aspect, the invention encompasses the treatment of a patient who has a neoplasm, such as a cancer, by administration of a compound of the formula (I) in combination with an antiproliferative agent. Suitable anti-proliferative agents encompass those provided in Table 1.

Above and below, all temperatures are indicated in ° C. In the following examples, “conventional work-up” means: if necessary, water is added, the pH is adjusted, if necessary, to values between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. Rt values are determined by HPLC using eluents mentioned.

Mass spectrometry (MS): EI (electron impact ionisation) M⁺
FAB (fast atom bombardment) (M+H)⁺
ESI (electrospray ionisation) (M+H)⁺
   APCI-MS (atmospheric pressure chemical ionisation—mass spectrometry) (M+H)+

### Analytical HPLC and LC/MS Methods

A HPLC method: 1_{—}100_{—}2 Speed (instrument: LaChrom)
Column: Chromolith Performance RP18e 100-3 mm
Flow rate: 2 ml/min (pump: L-7100)
Solvent A: water+0.01% of TFA
Solvent B: acetonitrile+0.01% of TFA
Wavelength: 220 nm (detector: L-7455)
Gradient: 0-0.2 min. 100% of A, 0.2-3.7 min. to 100% of B, 3.7-4.4 min. 100% of B, 4.5-5.0 min. 100% of A
   B HPLC/MS method: SOP 2222 (instrument: Waters)
Column: Chromolith Flash RP18e 25-2 mm
Flow rate: 2.4 ml/min (pump: Waters 1525 Binary HPLC Pump)
Solvent A: water+0.01% of TFA
Solvent B: acetonitrile+0.01% of TFA
Wavelength: 254 nm (detector: Waters 2488 Mux-UV Detector)
Gradient: 0-8 min 2% to 100% of B.
   C LC-MS method: polar.M (instrument: Agilent 1100 Series)
Column: Chromolith Speed Rod RP18e-50-4.6
Flow rate: 2.4 ml/min
Solvent A: water+0.05% of HCOOH
Solvent B: acetonitrile+0.04% of HCOOH
WL: 220 nm
Gradient: 0-2.8 min: 4% of B to 100% of B, 2.8-3.3 min:100% of B
   D HPLC method: 1_{—}100_{—}2 (instrument: LaChrom)
Column: Chromolith Performance RP18e 100-3 mm
Flow rate: 2 ml/min (pump: L-7100)
Solvent A: water+0.05% of CHOOH
Solvent B: acetonitrile+0.04% of CHOOH
Wavelength: 220 nm (detector: L-7455)
Gradient: 0-0.2 min: 99% of A, 0.2-3.8 min: 99% of A→100% of B,
3.8-4.4 min: 100% of B, 4.4-4.5 min: 100% of B→99% of A,
4.5-5.1 min: 99°/0 of A
   E HPLC/MS method (polar) (instrument: Agilent 1100 Series)
Solvent A: water+0.05% of formic acid
Solvent B: acetonitrile+0.04% of formic acid
Flow: 2.4 ml/min, wavelength: 220 nm
Gradient: 0.0 min 4% of B
   2.8 min 100% of B
   3.3 min 100% of B
   3.4 min 4°/0 of B
Column: Chromolith® Speed ROD RP-18e 50-4.6 mm
   F SFC method (ChiracelOJ-H 20% MOH) (instrument: Berger Instruments)
Solvent: carbon dioxide+20% of methanol
Flow: 5 ml/min, wavelength: 220 nm
Gradient: isocratic
Column: ChiracelOJ-H
   G HPLC/MS method (DMSO) (instrument: Agilent 1100 Series)
Solvent A: water+0.05% of formic acid
   Solvent B: acetonitrile+0.04% of formic acid
   Flow: 2.4 ml/min, wavelength: 220 nm
   Gradient: 0.0 min 5% of B
      0.5 min 5% of B
      2.8 min 100% of B
      3.5 min 100% of B
Display of the peaks in report only from 0.8 min
   Column: Chromolith® Speed ROD RP-18e 50-4.6 mm
      H HPLC/MS method (DMSO) (instrument: Waters Acquity HPLC® with PDA and ELSD and Waters SQD (ESI+/− and APCI+/−))
Solvent A: 99.9% of acetonitrile+0.1% of TFA
Solvent B: 99.9% of water+0.1% of TFA
Flow: 2 ml/min, wavelength: 256 nm
Gradient: 0.0 min 95% of B
   8.0 min 0% of B
   8.1 min 90% of B
   8.5 min 95% of B
   11.0 min 95% of B
      Column: Waters XBridge™ C8 3.5 μm; 4.6×50 mm column; Part No. 186003053
      I HPLC/MS method (DMSO) (instrument: Waters 1525 Binary HPLC Pump, Waters In-Line Degasser AF, Waters 2777 Sample Manager, Waters 2488 Mux-UV Detector, Waters 2420 ELS Detector, Waters ZQ-MUX)
Solvent A: 99.9% of acetonitrile+0.1% of formic acid
Solvent B: 99.9% of water+0.1% of formic acid
Flow: 0.8 ml/min, wavelength: 254 nm
Gradient: 0.0 min 95% of B
   1.7 min 0°/0 of B
   3.0 min 0% of B
   3.01 min 100% of B
   6.25 min 95°/0 of B

### Column: Chromolith® Flash RP-18e (25-2 mm)

Prep. HPLC method: 1_{—}10_{—}10_{—}50
(instrument: Agilent 1100 Series)

### Column: Chromolith Prep Rod RP18e

Flow rate: 50 ml/min
Solvent A: water+0.1% of TFA
Solvent B: acetonitrile+0.1% of TFA

### WL: 220 nm

Gradient: in 10 min from 1 to 10% of acetonitrile, collect from 2 to 11 min
Prep. HPLC method: 25_{—}50_{—}10
(instrument: Agilent 1100 Series)

### Column: Chromolith Prep Rod RP18e

Flow rate: 50 ml/min
Solvent A: water+0.1% of TFA
Solvent B: acetonitrile+0.1% of TFA

### WL: 220 nm

Gradient: in 2 min from 1 to 25% of acetonitrile, from 2 to 10 min to 50% of acetonitrile, collect from 2 to 11 min
Prep. HPLC method: 30_{—}60_{—}10
(instrument: Agilent 1100 Series)

### Column: Chromolith Prep Rod RP18e

Flow rate: 50 ml/min
Solvent A: acetonitrile+0.1% of TFA
Solvent B: water+0.1% of TFA

### WL: 220 nm

Gradient: in 2 min from 1-30% of acetonitrile, from 2 to 8 min to 60% of acetonitrile, collect from 2 min to 11 min
Prep. HPLC method 20_{—}40_{—}10
(instrument: Agilent 1100 Series)

### Column: Chromolith Prep Rod RP18e

Flow rate: 50 ml/min
Solvent A: acetonitrile+0.1% of TFA
Solvent B: water+0.1% of TFA

### WL: 220 nm

Gradient: from 1-20% of acetonitrile in 2 min, from 20-40% of acetonitrile in a further 8 min, collect from 2 min to 11 min
Prep. HPLC method: method 5_{—}70_{—}10
(instrument: Agilent 1100 Series)

### Column: Chromolith Prep Rod RP18e

Flow rate: 50 ml/min
Solvent A: acetonitrile+0.1% of formic acid
Solvent B: water+0.1% of formic acid

### WL: 220 nm

Gradient: from 5-70% of ACN in 15 min
Prep. HPLC method: 1_{—}60_{—}10
(instrument: Agilent 1100 Series)

### Column: Chromolith Prep Rod RP18e

Flow rate: 50 ml/min
Solvent A: acetonitrile+0.1% of formic acid
Solvent B: water+0.1% of formic acid

### WL: 220 nm

Gradient: from 1-60% of ACN in 16 min
Prep. HPLC method: 25_{—}50_{—}10_{—}50 ml_empfind_o_equi.M
(instrument: Agilent 1100 Series)

### Column: Chromolith Prep Rod RP18e

Flow rate 50 ml/min
Solvent A: acetonitrile+0.1% of TFA
Solvent B: water+0.1% of TFA

### WL: 220 nm

Gradient: from 1-25% in 2 min., 25-50% of solvent B in 8 minutes, collect from 2-11 min
Prep. HPLC method: 15_{—}35_{—}10_{—}50 ml_normal_o_equi.M
(instrument: Agilent 1100 Series)

### Column: Chromolith Prep Rod RP18e

Flow rate: 50 ml/min
Solvent A: acetonitrile+0.1% of TFA
Solvent B: water+0.1% of TFA

### WL: 220 nm

Gradient: from 1-15% of ACN in 2 min, from 15-35% of ACN in 8 min, collect from 2 min to 11 min

### Companion Method 1:

RediSep column: 40 g of silica
Detection wavelength: 254 nm
Flow rate: 40 ml/min

### Conditioning-volume: 120.0 ml

Run time 30.0 min
Eluent A: A1 cyclohexane
Eluent B: B1 ethyl acetate

| Duration | Per cent of B | Eluent B |
|---|---|---|
| 0.0 | 0.0 | B1 ethyl acetate |
| 1.3 | 0.0 | B1 ethyl acetate |
| 18.7 | 50.0 | B1 ethyl acetate |
| 4.0 | 50.0 | B1 ethyl acetate |
| 0.0 | 50.0 | B1 ethyl acetate |
| 5.0 | 50.0 | B1 ethyl acetate |
| 1.0 | 50.0 | B1 ethyl acetate |

### EXAMPLE 1

### Preparation of 7-(4-chloro-2-fluorophenyl)-2-(3,3-dimethylbutyryl)-1,3,4,11a-tetrahydro-2H,10H-2,4-a,10-triazadibenzo[a,d]cycloheptene-5,11-dione (“10”)

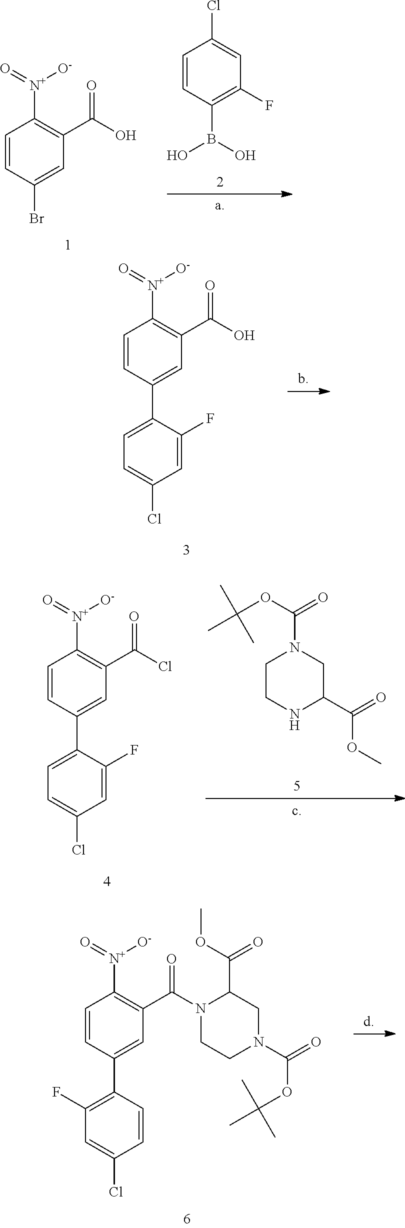

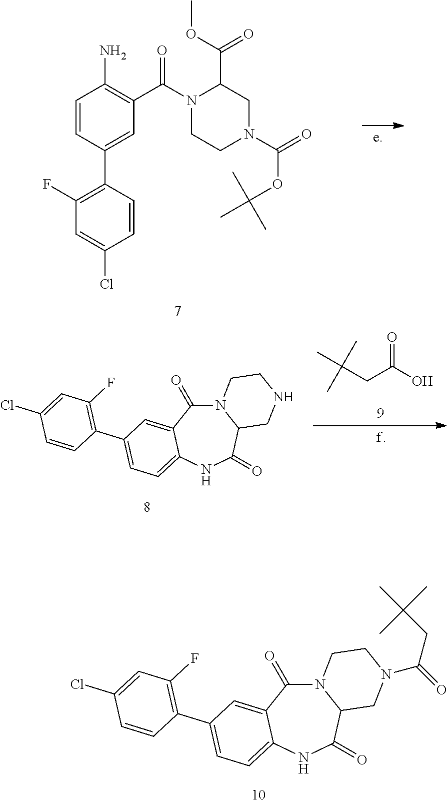

a. Preparation of 4′-chloro-2′-fluoro-4-nitrobiphenyl-3-carboxylic acid (3)

2.5 g (10 mmol) of starting material 1, 1.8 g (10 mmol) of starting material 2, and 2.9 g (35 mmol) of sodium hydrogencarbonate in 10 ml of water and 120 ml of ethylene glycol dimethyl ether are initially introduced in a 100 ml multinecked flask, and the flask is flushed with nitrogen. 0.5 g (0.4 mmol) of tetrakis(triphenylphosphine)palladium(0) are then added, and the reaction mixture is heated at 90° C. for 14 h. The solution is acidified to pH 4, and the solvent is removed. The residue is taken up in 60 ml of water and extracted with ethyl acetate. The combined organic phases are washed with water, dried over sodium sulfate, and the solvent is removed, The solid residue which remains is stirred with acetonitrile, filtered off with suction and dried, giving the product (900 mg, 3 mmol, 31% yield) as white crystals (mass: [M⁺−(OH)]=278; RT 2.96 min, HPLC method 1_{—}100_{—}2_Speed).

b. Preparation of 4′-chloro-2′-(fluoro-4-nitrobiphenyl-3-carbonyl chloride (4)

Starting material 3 (900 mg, 3 mmol) is initially introduced in 30 ml of dichloro-methane. 1.4 ml (16 mmol) of oxalyl chloride and one drop of DMF are then added with stirring.

The mixture is stirred for 14 h. The solvent is distilled off, and the crystalline residue (900 mg, 2.9 mmol. 94%) is reacted further without further purification.

c. Preparation of 1-tert-butyl 3-methyl 4-(4′-chloro-2′-fluoro-4-nitrobiphenyl-3-carbonyl)piperazine-1,3-dicarboxylate (6)

Starting material 5 (708 mg, 2.9 mmol) in 20 ml of dichloromethane is initially introduced in a 50 ml multinecked flask with dropping funnel, thermometer and N₂ inlet lube, and 1.7 ml of DIPEA are added. The solution is cooled to 0° C., and a solution of 900 mg (3 mmol) of starting material 4 in 10 ml of dichloro-methane is added dropwise over the course of 15 min with stirring. The ice bath is then removed, and the mixture is stirred for a further one hour. Water is added to the reaction mixture, the organic phase is separated off, dried over sodium sulfate, and the solvent is removed. The residue is filtered absorptively through a silica-gel column with ethyl acetate, and the filtrate is evaporated to dryness. The desired product 6 is obtained in a yield of 80% (1.5 g, 2.3 mmol) as solid (mass: [M*−(^{t}Bu)]=266; RT 3.44 min. HPLC method 1_{—}100_{—}2_Speed).

d. Preparation of 1-tert-butyl 3-methyl 4-(4-amino-4′-chloro-2′-fluoro-biphenyl-3-carbonyl)piperazine-1,3-dicarboxylate (7)

Starting material 6 (1.5 g, 2.3 mmol) is hydrogenated in 200 ml of methanol using 1 g of 5% Pd/C (50.5% of water). The catalyst is filtered off, and the solvent is removed. The product 7 (1.3 g, 2.3 mmol, 92%) is reacted further without further purification.

e. Preparation of 7-(4-chloro-2-fluorophenyl)-1,3,4,11a-tetrahydro-2H,10H-2.4a,10-triazadibenzo[a,d]cycloheptene-5,1-dione (8)

Starting material 7 (1,3 g, 3 mmol) is stirred at 110° C. for 3 h in 40 ml of glacial acetic acid, 50 ml of 4N HCL in dioxane are subsequently added at room temperature, and the reaction mixture is stirred for a further 3 h. The mixture is evaporated to dryness, the residue is dissolved in water, the pH was adjusted to 9 using 1N NaOH, and the mixture is extracted with dichloromethane. The combined organic phases are washed with water, dried over sodium sulfate, filtered, and the filtrate is evaporated to dryness. The residue is dissolved in 20 ml of methanol. The desired product 8 (530 mg, 1.5 mmol, 77%) is obtained as colourless crystals by addition of 150 ml of diethyl ether. (Mass [M+]=360; RT 2.48 min, HPLC method 1_{—}100_{—}2_Speed).

f. Preparation of 7-(4-chloro-2-fluorophenyl)-2-(3,3-dimethylbutyryl)-1,3,4,11a-tetrahydro-2H,10H-2,4a,10-triazadibenzo[a,d]cycloheptene-5,11-dione (10)

Starting material 8 (100 mg, 0.3 mmol) and 3,3-dimethytbutyric acid 9 (32.3 mg, 0.3 mmol) are dissolved in 1 ml of DMF. 63.3 mg (0.33 mmol) of DAPECI and 50.5 mg (0.33 mmol) of HOBT hydrate are added, and the mixture is stirred at room temperature for 3 h. The solvent is removed, and the residue is purified via an HPLC apparatus (method 30-60-10; 50 ml/min), thus giving the desired product 10 (28 mg, 21% yield, 94% content) as amorphous solid (mass: [M+]=458; RT 3.35 min, HPLC method 1_{—}100_{—}2_Speed); ¹H NMR (500 MHz, DMSO-d₆) δ [ppm] 10.64 (m, 1H), 7.93 (s, 1H), 7.76-7.71 (m, 1H), 7.68-7.54 (m, 2H), 7.41 (d, J=8.4, 1H), 7.24 (d, J=8.5, 1H), 4.31 (m, 1H), 4.23-3.86 (m, 2H), 3.84-3.71 and 3.65 (2×m, 2H), 3.60-3.41 (m, 2H), 2.40-2.14 (m, 2H), 1.01 (m, 9H).

The following compounds are obtained analogously

| Compound | | HPLC method; |
|---|---|---|
| No. | Name and/or structure | RT [min] |
| “A1” | (R)-7-(4-Chlorophenyl)-2-((S)-2-hydroxy-2- phenylacetyl)-1,3,4,11a-tetrahydro-2H,10H- 2,4a,10-triazadibenzo[a,d]cycloheptene-5,11-dione | B; 1.61 |
| | 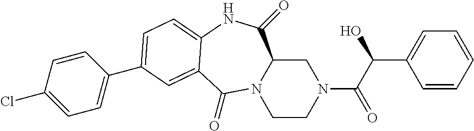 | |
| “A2” | 2-[(R)-7-(2-Fluorophenyl)-5,11-dioxo- 3,4,5,10,11,11a-hexahydro-1H-2,4a,10-triaza- dibenzo[a,d]cyclohepten-2-yl]-2-oxoacetamide | B; 1.45 |
| | 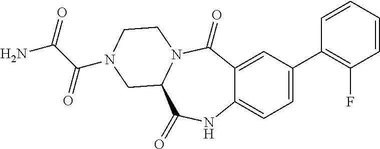 | |
| “A3” | (R)-2-((S)-2-Hydroxy-2-phenylacetyl)-7-thiophen-2- yl-1,3,4,11a-tetrahydro-2H,10H-2,4a,10-triaza- dibenzo[a,d]cycloheptene-5,11-dione | B; 1.55 |
| | 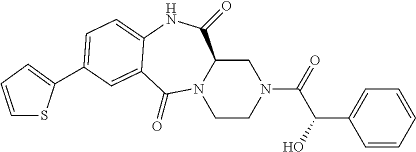 | |
| “A4” | 2-((R)-7-Furan-2-yl-5,11-dioxo-3,4,5,10,11,11a- hexahydro-1H-2,4a,10-triazadibenzo[a,d]cyclo- hepten-2-yl)-2-oxoacetamide | B; 1.34 |
| | 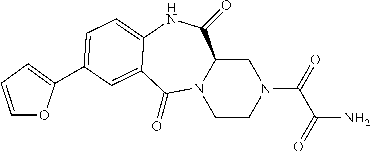 | |
| “A5” | (R)-7-(4-Chlorophenyl)-2-((R)-thiazolidine-4- carbonyl)-1,3,4,11a-tetrahydro-2H,10H-2,4a,10- triazadibenzo[a,d]cycloheptene-5,11-dione | A; 2.45 |
| | 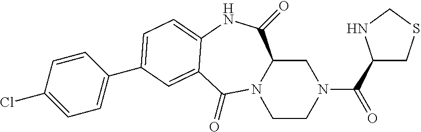 | |
| “A6” | (R)-7-Furan-2-yl-2-(2-hydroxyacetyl)-1,3,4,11a- tetrahydro-2H,10H-2,4a,10-triazadibenzo[a,d]- cycloheptene-5,11-dione | B; 1.35 |
| | 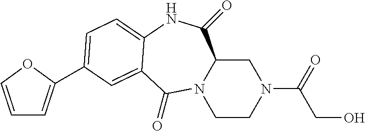 | |
| “A7” | (R)-2-(3,3-Dimethylbutyryl)-7-(2-fluorophenyl)- 1,3,4,11a-tetrahydro-2H,10H-2,4a,10-triaza- dibenzo[a,d]cycloheptene-5,11-dione | A; 3.11 |
| | 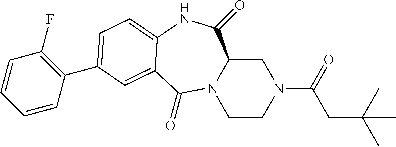 | |
| “A8” | (R)-2-(2-Hydroxyacetyl)-7-thiophen-2-yl-1,3,4,11a- tetrahydro-2H,10H-2,4a,10-triazadibenzo[a,d]- cycloheptene-5,11-dione | B; 1.3 |
| | 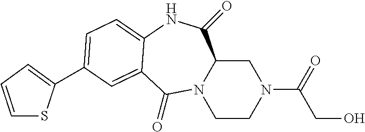 | |
| “A9” | (R)-7-(4-Chlorophenyl)-2-(4-dimethylamino- benzoyl)-1,3,4,11a-tetrahydro-2H,10H-2,4a,10- triazadibenzo[a,d]cycloheptene-5,11-dione | B; 1.71 |
| | 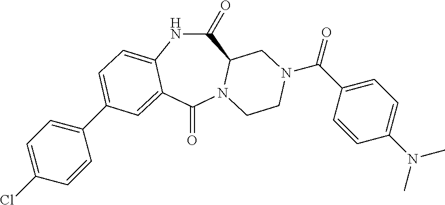 | |
| “A10” | (R)-7-(2-Fluorophenyl)-2-((R)-thiazolidine-4- carbonyl)-1,3,4,11a-tetrahydro-2H,10H-2,4a,10- triazadibenzo[a,d]cycloheptene-5,11-dione | A; 2.47 |
| | 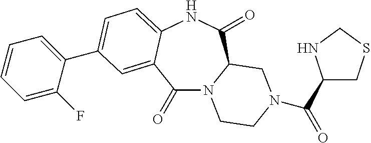 | |
| “A11” | (R)-2-(3-Fluorobenzoyl)-7-(4-methoxyphenyl)- 1,3,4,11a-tetrahydro-2H,10H-2,4a,10-triaza- dibenzo[a,d]cycloheptene-5,11-dione | B; 1.59 |
| | 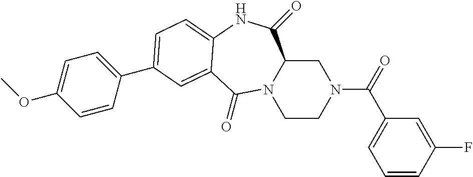 | |
| “A12” | 7-(2-Fluorophenyl)-2-((R)-thiazolidine-4-carbonyl)- 1,3,4,11a-tetrahydro-2H,10H-2,4a,10-triaza- dibenzo[a,d]cycloheptene-5,11-dione | A; 2.47 |
| | 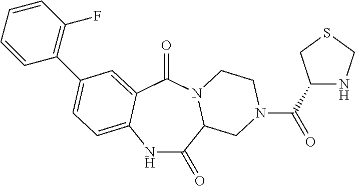 | |
| “A13” | 2-(3,3-Dimethylbutyryl)-7-(2-fluorophenyl)- 1,3,4,11a-tetrahydro-2H,10H-2,4a,10-triaza- dibenzo[a,d]cycloheptene-5,11-dione | A; 3.11 |
| | 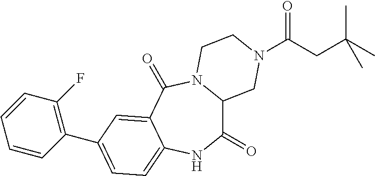 | |
| “A14” | 2-(1H-Benzotriazole-5-carbonyl)-7-(2-fluorophenyl)- 1,3,4,11a-tetrahydro-2H,10H-2,4a,10-triaza- dibenzo[a,d]cycloheptene-5,11-dione | A; 2.75 |
| | 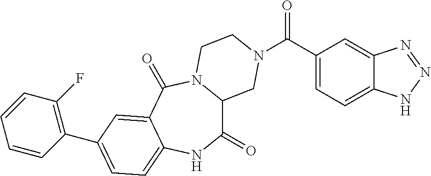 | |
| “A15” | 7-(2-Fluorophenyl)-2-(pyridine-2-carbonyl)- 1,3,4,11a-tetrahydro-2H,10H-2,4a,10-triaza- dibenzo[a,d]cycloheptene-5,11-dione | A; 2.77 |
| | 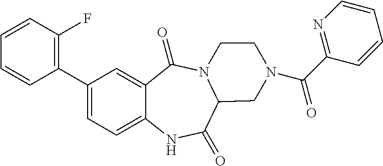 | |

¹H NMR (500 MHz, DMSO-d₆) δ [ppm] 10.67 and 10.55 (s, 1H), 8.87 and 8.79 (2 x d, J = 5.0, 1H), 8.41 and 8.25 (2 x t, J = 7.8, 1H), 8.16-7.70 (m, 4H), 7.57 (t, J = 7.1, 1H), 7.44 (m, 1H), 7.37-7.20 (m, 3H), 4.59-3.65 (m, 9H) [rotamer mixture]

| | | |
|---|---|---|
| “A16” | (S)-7-(2-Fluorophenyl)-2-((R)-thiazolidine-4- carbonyl)-1,3,4,11a-tetrahydro-2H,10H-2,4a,10- triazadibenzo[a,d]cycloheptene-5,11-dione | A; 2.47 |
| | 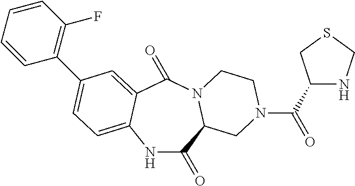 | |
| “A17” | (S)-2-(3,3-Dimethylbutyryl)-7-(2-fluorophenyl)- 1,3,4,11a-tetrahydro-2H,10H-2,4a,10-triaza- dibenzo[a,d]cycloheptene-5,11-dione | A; 3.11 |
| | 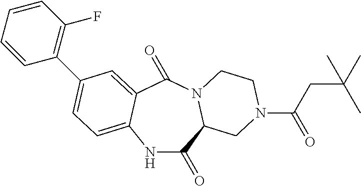 | |
| “A18” | 2-(3,3-Dimethylbutyryl)-7-(4-trifluoromethylphenyl)- 1,3,4,11a-tetrahydro-2H,10H-2,4a,10-triaza- dibenzo[a,d]cycloheptene-5,11-dione | A; 3.4 |
| | 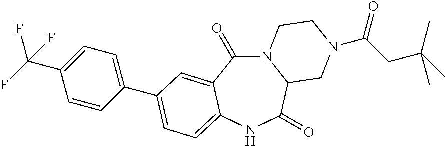 | |
| “A19” | 7-(3,5-Dichlorophenyl)-2-(3,3-dimethylbutyryl)- 1,3,4,11a-tetrahydro-2H,10H-2,4a,10-triaza- dibenzo[a,d]cycloheptene-5,11-dione | A; 3.51 |
| | 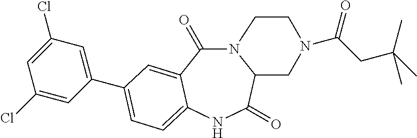 | |
| “A20” | 7-(2-Fluorophenyl)-2-(3-pyridin-2-ylpropionyl)- 1,3,4,11a-tetrahydro-2H,10H-2,4a,10-triaza- dibenzo[a,d]cycloheptene-5,11-dione | A; 2.45 |
| | 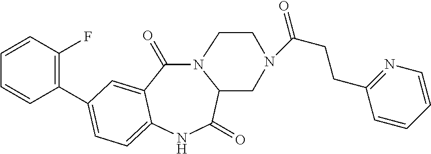 | |

¹H NMR (500 MHz, DMSO-d₆) δ [ppm] 10.65 (m, 1H), 8.71 (m, 1H), 8.30 (br. s, 1 H), 7.94 (m, 1H), 7.84 (m, 1H), 7.78-7.66 (m, 2H), 7.59-7.50 (m, 1H), 7.44 (m, 1H), 7.37-7.28 (m, 2H), 7.24 (dd, J = 8.5, 3.0, 1H), 4.40- 2.53 (m, 11H) [rotamer mixture]

| | | |
|---|---|---|
| “A21” | 7-(4-Chlorophenyl)-2-((S)-piperidine-2-carbonyl)- 1,3,4,11a-tetrahydro-2H,10H-2,4a,10-triaza- dibenzo[a,d]cycloheptene-5,11-dione | A; 2.43 |
| | 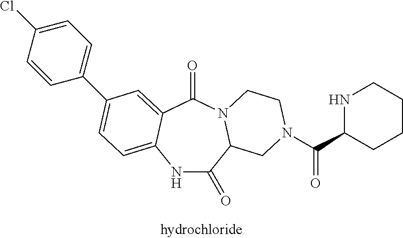 | |
| “A22” | 7-(4-Chlorophenyl)-2-(2-methanesulfonylacetyl)- 1,3,4,11a-tetrahydro-2H,10H-2,4a,10-triaza- dibenzo[a,d]cycloheptene-5,11-dione | A; 2.63 |
| | 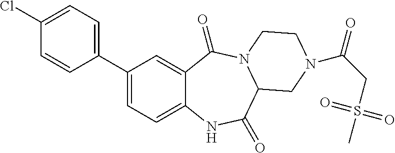 | |

¹H NMR (500 MHz. DMSO-d₆) δ [ppm] 10.66 (m, 1H), 8.03 (dd, J = 8.4, 2.3, 1H), 7.88 (m, 1H), 7.73 (m, 2H), 7.54 (d, J = 8.5, 2H), 7.24 (dd, J = 8.5, 3.7, 1H), 4.45-3.42 (m, 9H), 3.12 (m, 3H) [rotamer mixture]

| | | |
|---|---|---|
| “A23” | 7-(4-Chlorophenyl)-2-(1-methyl-3-propyl-1H- pyrazole-4-carbonyl)-1,3,4,11a-tetrahydro-2H,10H- 2,4a,10-triazadibenzo[a,d]cycloheptene-5,11-dione | A; 3.07 |
| | 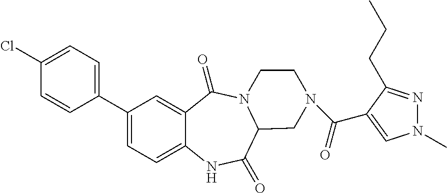 | |
| “A24” | 7-(4-Chlorophenyl)-2-(2-cyclohexylacetyl)- 1,3,4,11a-tetrahydro-2H,10H-2,4a,10-triaza- dibenzo[a,d]cycloheptene-5,11-dione | A; 3.47 |
| | 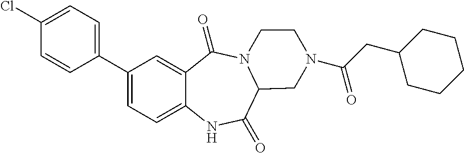 | |
| “A25” | 7-(4-Chlorophenyl)-2-(2-cyclopentylacetyl)- 1,3,4,11a-tetrahydro-2H,10H-2,4a,10-triaza- dibenzo[a,d]cycloheptene-5,11-dione | A; 3.37 |
| | 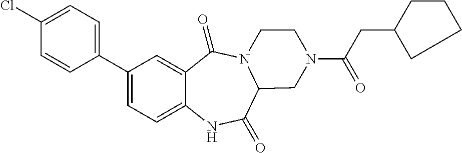 | |
| “A26” | 7-(4-Chlorophenyl)-2-[3-(1-methyl-1H-pyrazol-4-yl)- propionyl]-1,3,4,11a-tetrahydro-2H,10H-2,4a,10- triazadibenzo[a,d]cycloheptene-5,11-dione | A; 2.92 |
| | 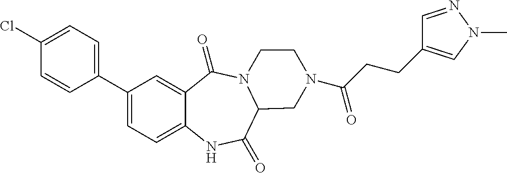 | |

¹H NMR (500 MHz, DMSO-d₆) δ [ppm] 10.62 (m, 1H), 8.02 (s, 1H), 7.86 (dd, J = 8.4, 2.3, 1H), 7.75-7.68 (m, 2H), 7.56-7.50 (m, 2H), 7.46 (m, 1H), 7.28-7.19 (m, 2H), 4.35-4.27 (m, 1H), 4.21-3.43 (m, 9H), 2.78-2.52 (m, 4H)

| | | |
|---|---|---|
| “A27” | 7-(4-Chloro-2-fluorophenyl)-2-(2-thiophen-3-yl- acetyl)-1,3,4,11a-tetrahydro-2H,10H-2,4a,10- triazadibenzo[a,d]cycloheptene-5,11-dione | A; 3.26 |
| | 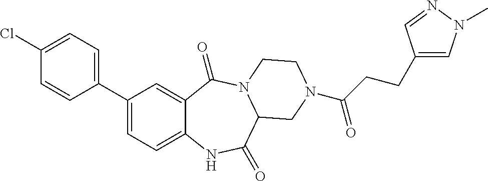 | |

¹H NMR (500 MHz, DMSO-d₆) δ [ppm] 10.69 (m, 1H), 7.93 (s, 1H), 7.79- 7.68 (m, 1H), 7.67-7.52 (m, 2H), 7.49-7.37 (m, 2H), 7.32-7.20 (m, 2H), 7.07-6.96 (m, 1H), 4.39-3.43 (m, 9H)

| | | |
|---|---|---|
| “A28” | 7-(4-Chloro-2-fluorophenyl)-2-(2-cyclopropylacetyl)- 1,3,4,11a-tetrahydro-2H,10H-2,4a,10-triaza- dibenzo[a,d]cycloheptene-5,11-dione | A; 3.13 |
| | 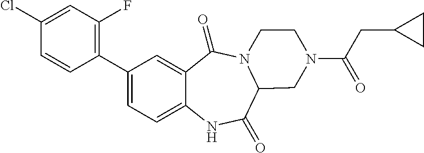 | |

¹H NMR (500 MHz, DMSO-d₆) δ [ppm] 10.65 (m, 1H), 7.93 (s, 1H), 7.73 (d, J = 8.4, 1H), 7.67-7.53 (m, 2H), 7.41 (dd, J = 8.3, 1.6, 1H), 7.24 (dd, J = 8.4, 2.7, 1H), 4.32 (m, 1H), 4.18-3.91 (m, 2H), 3.83-3.38 (m, 4H), 2.45- 2.16 (m, 2H), 1.06-0.92 (m, 1H), 0.50-0.39 (m, 2H), 0.18-0.05 (m, 2H)

| | | |
|---|---|---|
| “A29” | 7-(4-Chlorophenyl)-2-(2-pyridin-2-ylacetyl)- 1,3,4,11a-tetrahydro-2H,10H-2,4a,10-triaza- dibenzo[a,d]cycloheptene-5,11-dione | A; 2.62 |
| | 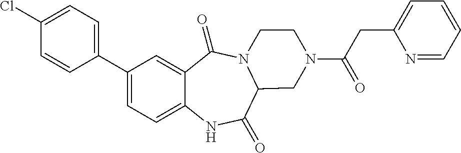 | |
| “A30” | 7-(4-Chloro-2-fluorophenyl)-2-(2-pyridin-2-ylacetyl)- 1,3,4,11a-tetrahydro-2H,10H-2,4a,10-triaza- dibenzo[a,d]cycloheptene-5,11-dione | A; 2.61 |
| | 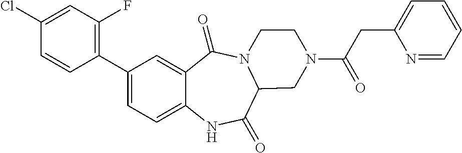 | |
| “A31” | 7-(4-Chloro-2-fluorophenyl)-2-phenylacetyl- 1,3,4,11a-tetrahydro-2H,10H-2,4a,10-triaza- dibenzo[a,d]cycloheptene-5,11-dione | A; 3.29 |
| | 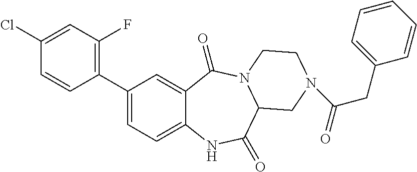 | |
| “A32” | 7-(4-Chlorophenyl)-2-phenylacetyl-1,3,4,11a- tetrahydro-2H,10H-2,4a,10-triazadibenzo[a,d]- cycloheptene-5,11-dione | D, 3.21 |
| | 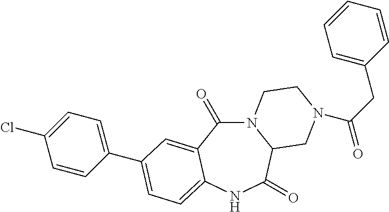 | |

¹H NMR (500 MHz, DMSO-d₆) δ [ppm] 10.71-10.58 (m, 1H), 8.01 (dd, J = 5.8, 2.3, 1H), 7.86 (ddd, J = 8.2, 5.9, 2.3, 1H), 7.72 (m, 2H), 7.53 (d, J = 8.5, 2H), 7.33-7.15 (m, 6H), 4.36-3.42 (m, 9H) [rotamer mixture]

| | | |
|---|---|---|
| “A34” | 7-(4-Chlorophenyl)-2-((S)-2-hydroxy-3,3- dimethylbutyryl)-1,3,4,11a-tetrahydro-2H,10H- 2,4a,10-triazadibenzo[a,d]cycloheptene-5,11-dione | D; 3.11 |
| | 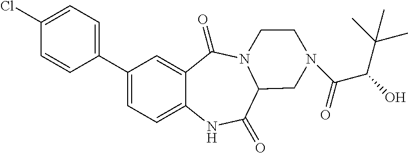 | |

¹H NMR (500 MHz, DMSO-d₆) δ [ppm] 10.64 (m, 1H), 8.02 (m, 1H), 7.93- 7.82 (m, 1H), 7.77-7.49 (m, 5H), 7.23 (m, 1H), 4.83-3.36 (m, 8H), 1.01- 0.79 (m, 9H) [rotamer mixture]

| | | |
|---|---|---|
| “A35” | 7-(4-Chlorophenyl)-2-(2-oxo-2-phenylacetyl)- 1,3,4,11a-tetrahydro-2H,10H-2,4a,10-triaza- dibenzo[a,d]cycloheptene-5,11-dione | D; 3.35 |
| | 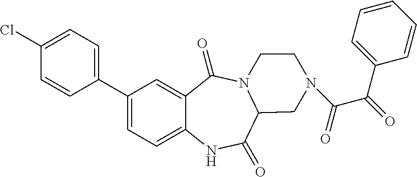 | |
| “A36” | 7-(4-Chlorophenyl)-2-(thiazole-4-carbonyl)- 1,3,4,11a-tetrahydro-2H,10H-2,4a,10-triaza- dibenzo[a,d]cycloheptene-5,11-dione | D; 2.93 |
| | 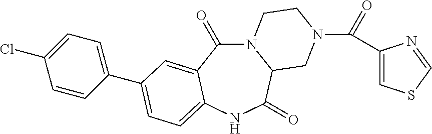 | |

¹H NMR (500 MHz, DMSO-d₆) δ [ppm] 10.60 (m, 1H), 9.16 (m, 1H), 8.24 (d, J = 1.9, 1H), 8.02 (s, 1H), 7.84 (m, 1H), 7.71 (d, J = 8.5, 2H), 7.52 (d, J = 8.5, 2H), 7.20 (m, 1H), 4.51-4.23 (m, 2H), 4.18-3.58 (m, 5H)

| | | |
|---|---|---|
| “A37” | 2-(5-Amino-2-methylthiazole-4-carbonyl)-7-(4- chlorophenyl)-1,3,4,11a-tetrahydro-2H,10H- 2,4a,10-triazadibenzo[a,d]cycloheptene-5,11-dione | D; 3.11 |
| | 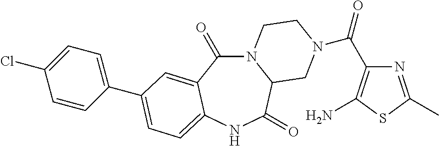 | |
| “A38” | 7-(4-Chlorophenyl)-2-[2-(4-chlorophenyl)-2- hydroxyacetyl]-1,3,4,11a-tetrahydro-2H,10H- 2,4a,10-triazadibenzo[a,d]cycloheptene-5,11-dione | D; 3.25 |
| | 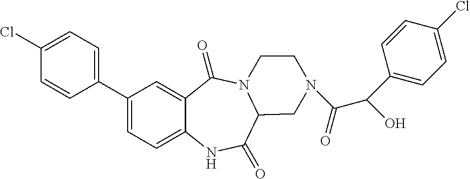 | |
| “A39” | 7-(4-Chlorophenyl)-2-(2-hydroxy-2- phenylpropionyl)-1,3,4,11a-tetrahydro-2H,10H- 2,4a,10-triazadibenzo[a.d]cycloheptene-5,11-dione | D; 3.16 |
| | 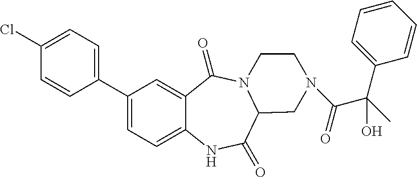 | |
| “A40” | 7-(4-Chlorophenyl)-2-[2-(2-chlorophenyl)-2- hydroxyacetyl]-1,3,4,11a-tetrahydro-2H,10H- 2,4a,10-triazadibenzo[a,d]cycloheptene-5,11-dione | D; 3.22 |
| | 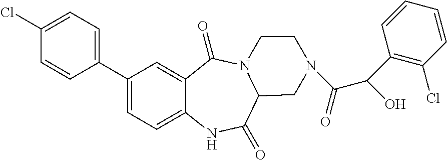 | |
| “A41” | 7-(4-Chlorophenyl)-2-[2-(3-chlorophenyl)-2- hydroxyacetyl]-1,3,4,11a-tetrahydro-2H,10H- 2,4a,10-triazadibenzo[a,d]cycloheptene-5,11-dione | D; 3.22 |
| | 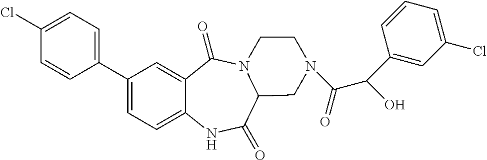 | |
| “A42” | 7-(4-Chlorophenyl)-2-(2-methoxy-2-phenylacetyl)- 1,3,4,11a-tetrahydro-2H,10H-2,4a,10-triaza- dibenzo[a,d]cycloheptene-5,11-dione | D; 3.14 |
| | 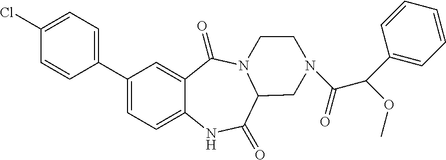 | |
| “A43” | 7-(4-Chlorophenyl)-2-((R)-2-hydroxy-2- phenylacetyl)-1,3,4,11a-tetrahydro-2H,10H- 2,4a,10-triazadibenzo[a,d]cycloheptene-5,11-dione | D; 3.13 |
| | 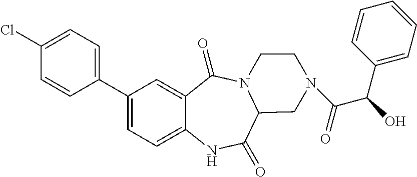 | |
| “A44” | 7-(4-Chlorophenyl)-2-(2-hydroxy-2-m-tolylacetyl)- 1,3,4,11a-tetrahydro-2H,10H-2,4a,10-triaza- dibenzo[a,d]cycloheptene-5,11-dione | D; 3.24 |
| | 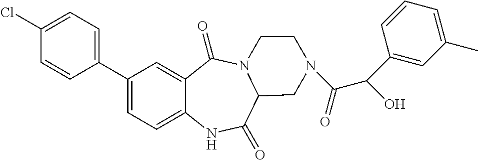 | |
| “A45” | 7-(4-Chlorophenyl)-2-[2-hydroxy-2-(3-trifluoro- methylphenyl)acetyl]-1,3,4,11a-tetrahydro-2H,10H- 2,4a,10-triazadibenzo[a,d]cycloheptene-5,11-dione | D; 3.35 |
| | 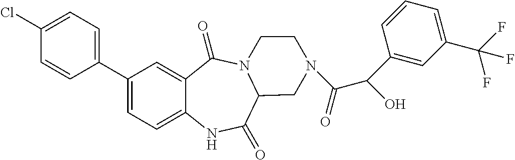 | |
| “A46” | 7-(4-Chlorophenyl)-2-(2-methyl-2-phenylpropionyl)- 1,3,4,11a-tetrahydro-2H,10H-2,4a,10-triaza- dibenzo[a,d]cycloheptene-5,11-dione | D; 346 |
| | 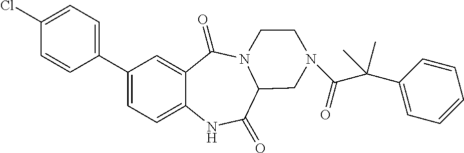 | |

¹H NMR (500 MHz, DMSO-d₆) δ [ppm] 8.01-7.91 (m, 2H), 7.85 (dd, J = 8.5, 2.2, 1H), 7.81-7.68 (m, 3H), 7.64-7.57 (m, 1H), 7.57-7.51 (m, 3H), 7.18 (m, 3H), 4.43-3.58 (m, 7H), 0.92 (m, 6H)

| | | |
|---|---|---|
| “A47” | 7-(4-Chlorophenyl)-2-[1-(4-chlorophenyl)- cyclopropanecarbonyl]-1,3,4,11a-tetrahydro- 2H,10H-2,4a,10-triazadibenzo[a,d]cycloheptene- 5,11-dione | D; 3.53 |
| | 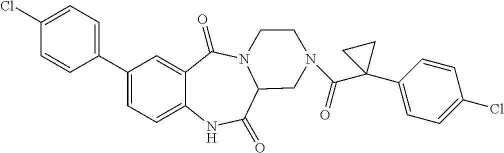 | |
| “A48” | N-Phenyl-7-(4-chlorophenyl)-5,11-dioxo- 3,4,5,10,11,11a-hexahydro-1H-2,4a,10-triaza- dibenzo[a,d]cycloheptene-2-carboxamide | D; 3.28 |
| | 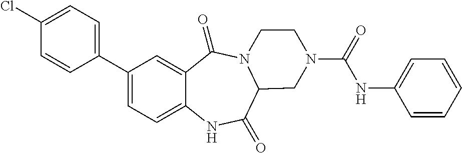 | |

¹H NMR (500 MHz, DMSO-d₆) δ [ppm] 11.04-10.64 (m, 2H), 8.05 (2 x d, J = 2.1, 1H), 7.98-7.84 (m, 3H), 7.73 (m, 2H), 7.60 (m, 1H), 7.55-7.46 (m, 4H), 7.24 (2 x d, 8.5, 1H), 4.62-3.67 (m, 7H) [rotamer mixture]

| | | |
|---|---|---|
| “A49” | 7-(4-Chlorophenyl)-2-(1-phenylcyclopropane- carbonyl)-1,3,4,11a-tetrahydro-2H,10H-2,4a,10- triazadibenzo[a,d]cycloheptene-5,11-dione | D; 3.36 |
| | 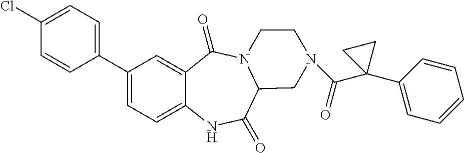 | |
| “A50” | 2-(2-Aminothiazole-4-carbonyl)-7-(4-chlorophenyl)- 1,3,4,11a-tetrahydro-2H,10H-2,4a,10-triaza- dibenzo[a,d]cycloheptene-5,11-dione | D; 2.86 |
| | 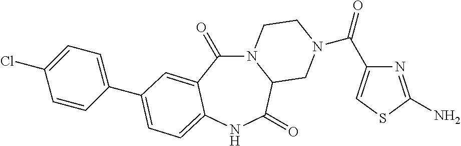 | |
| “A51” | 7-(4-Chlorophenyl)-2-(pyridine-2-carbonyl)- 1,3,4,11a-tetrahydro-2H,10H-2,4a,10-triaza- dibenzo[a,d]cycloheptene-5,11-dione | D; 2.93 |
| | 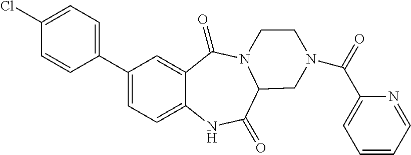 | |
| “A53” | 2-((R)-2-Amino-3,3-dimethylbutyryl)-7-(4-chloro- phenyl)-1,3,4,11a-tetrahydro-2H,10H-2,4a,10- triazadibenzo[a,d]cycloheptene-5,11-dione | D; 2.58 |
| | 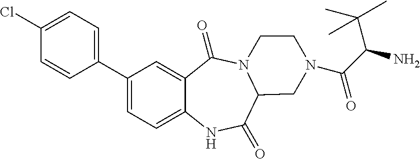 | |

¹H NMR (500 MHz, DMSO-d₆) δ [ppm] 11.04-10.44 (m, 1H), 8.12-7.82 (m, 4H), 7.77-7.67 (m, 2H), 7.59-7.49 (m, 2H), 7.25 (t, J = 8.4, 1H), 4.60- 3.17 (m, 8 H), 1.03 (m, 9H) [rotamer mixture]

| | | |
|---|---|---|
| “A54” | (S)-7-(4-Chlorophenyl)-2-((S)-2-hydroxy-3,3-di- methyl-1-oxobutyl)-1,3,4,11a-tetrahydro-2H,10H- 2,4a,10-triazadibenzo[a,d]cycloheptene-5,11-dione | D; 3.12 |
| | 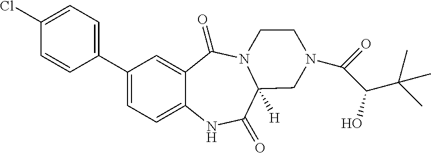 | |
| “A55” | N-{(R)-1-[7-(4-Chlorophenyl)-5,11-dioxo- 3,4,5,10,11,11a-hexahydro-1H-2,4a,10-triaza- dibenzo[a,d]cycloheptene-2-carbonyl]-2,2- dimethylpropyl}formamide | D; 2.67 |
| | 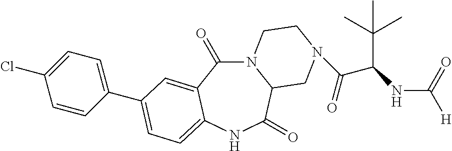 | |
| “A56” | (R)-7-(4-Chlorophenyl)-2-((S)-2-hydroxy-3,3-di- methyl-1-oxobutyl)-1,3,4,11a-tetrahydro-2H,10H- 2,4a,10-triazadibenzo[a,d]cycloheptene-5,11-dione | D; 3.17 |
| | 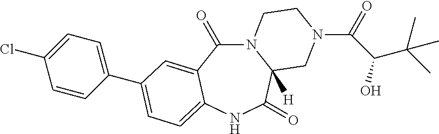 | |

¹H NMR (500 MHz, DMSO-d₆) δ [ppm] 10.67 and 10.57 (2 x s, 1H), 8.02 (dd, J = 8.8, 2.2, 1H), 7.87 (ddd, J = 8.4, 3.8, 2.4, 1H), 7.72 (dd, J = 8.5, 5.3, 2H), 7.53 (d, J = 8.1, 2H), 7.23 (dd, J = 8.4, 5.5, 1H), 4.59 (m, 1H), 4.42- 4.35 (m, 1H), 4.32-4.03 (m, 3H), 4.01-3.90 (m, 1H), 3.82 (m, 1H), 3.57 (m, 2H), 0.92 (m, 9H)

### EXAMPLE 2

### Preparation of 7-(3-chlorophenyl)-2-(3,3-dimethylbutyryl)-1,3,4,11a-tetrahydro-2H,10H-2,4-a,10-triazadibenzo[a,d]cycloheptene-5,11-dione (“17”)

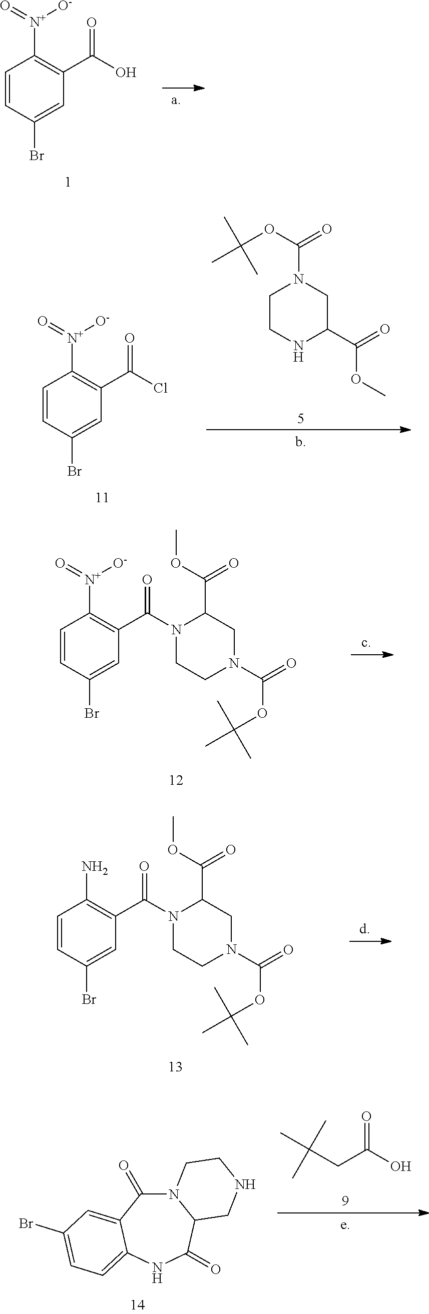

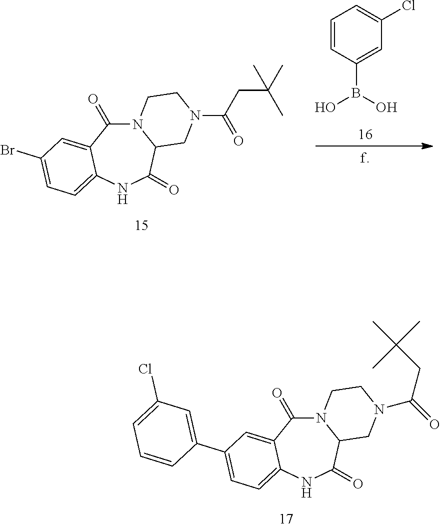

a. Preparation of 5-bromo-2-nitrobenzoyl chloride (11)

The desired product 11 are prepared quantitatively analogously to Example 1.b. from 3 g of starting material 1 (12.2 mmol), 5.5 ml (65 mmol) of oxalyl chloride and 1 ml of DMF in 30 ml of dichloromethane and are reacted further as amorphous residue (3.2 g. 12.1 mmol, 80% content) without further purification.

b. Preparation of 1-tert-butyl 3-methyl 4-(5-bromo-2-nitrobenzoyl)-piperazine-1,3-dicarboxylate (12)

Analogously to Example 1.c., starting material 5 (2 g, 8.2 mmol), starting material 11 (3.2 g, 12.1 mmol) and 7.2 ml of DIPEA(42.5 mmol) in 30 ml of dichloromethane give the desired product 12 after purification on the Companion, method 1, in a yield of 55% (3.2 g, 6.7 mmol) as colourless, crystalline solid (mass: [M⁺−(^{t}Bu)]=416; RT 3.43 min, HPLC method 1_{—}100_{—}2).

c. Preparation of 1-tert-butyl 3-methyl 4-(2-amino-5-bromobenzoyl)-piperazine-1,3-dicarboxylate (13)

Starting material 12 (3.1 g, 6.6 mmol) is hydrogenated in 30 ml of THF using 442 ml of hydrogen on 1.5 g of sponge nickel (pH-neutral, THF-moist), giving, after filtration and concentration in vacuo, the desired product 13 (2.6 g, 5.9 mmol, 90%) as amorphous solid (mass: [M⁺−(^{t}Bu)]=386; RT 3.29 min, HPLC method 1_{—}100_{—}2). It is reacted further without further purification.

d. Preparation of 7-bromo-1,3,4,11a-tetrahydro-2H10H-2,4a,10-triazadibenzo[a,d]cycloheptene-5,11-dione (14)
Analogously to Example 1.e., starting material 13 (2.2 g, 4.9 mmol), 50 ml of glacial acetic acid and 50 ml of 4N HCL in methanol give, after purification on the preparative HPLC (method 1_{—}10_{—}10_{—}50), the desired product 14 (200 mg, 0.65 mmol, 13%) as amorphous solid (mass [M+]=312; RT 1.73 min, HPLC method 1_{—}100_{—}2).
e. Preparation of 7-bromo-2-(3,3-dimethylbutyryl)-1.3.4,11a-tetrahydro-2H, 10H-2,4a, 10-triazadibenzo[a,d]cycloheptene-5,11-dione (15)

Analogously to Example 1.f., starting material 14 (200 mg, 0.6 mmol), 3,3-dimethylbutyric acid 9 (0.1 ml, 0.6 mmol), 106 mg of HOBT hydrate (0.8 mmol), 148 mg (0.8 mmol) of DAPECI in 2 ml of DMF give the desired product 15 (230 mg, 66% yield, 75% content) as pink amorphous solid, which is reacted further without further purification (mass: [M+]=409; RT 2.87 min, HPLC method 1_{—}100_{—}2).

f. Preparation of 7-(3-chlorophenyl)-2-(3,3-dimethylbutyryl)-1,3,4,11a-tetra-hydro-2H, 10H-2,4a, 10-triazadibenzo[a,d]cycloheptene-5,11-dione (17)

Analogously to Example 1.a., 230 mg (0.6 mmol) of starting material 15, 100 mg (0.6 mmol) of starting material 16, and 166 mg (2 mmol) of sodium hydrogencarbonate in 10 ml of water and 10 ml ot ethylene glycol dimethyl ether with 0.5 g (0.4 mmol) of tetrakis(triphenylphosphine)palladium(0) give the desired product 17 after purification via preparative HPLC (method 25_{—}50_{—}10) as colourless, crystalline solid (12.5 mg, 0.3 mmol, 5% yield; mass: [M*)]=440; RT 3.25 min, HPLC method 1_{—}100_{—}2).

### EXAMPLE 3

### Preparation of 1-[7-(2-fluorophenyl)-3,4,5,10,11,11a-hexahydro-1H-2,4-a,10-triazadibenzo[a,d]cyclohepten-2-yl]-3,3-dimethylbutan-1-one (“25”) and 2-(3,3-dimethylbutyryl)-7-(2-fluorophenyl)-1,3,4,5,10,11a-hexahydro-2H-2,4-a,10-triazadibenzo[a,d]cyclohepten-11-one (“26”)

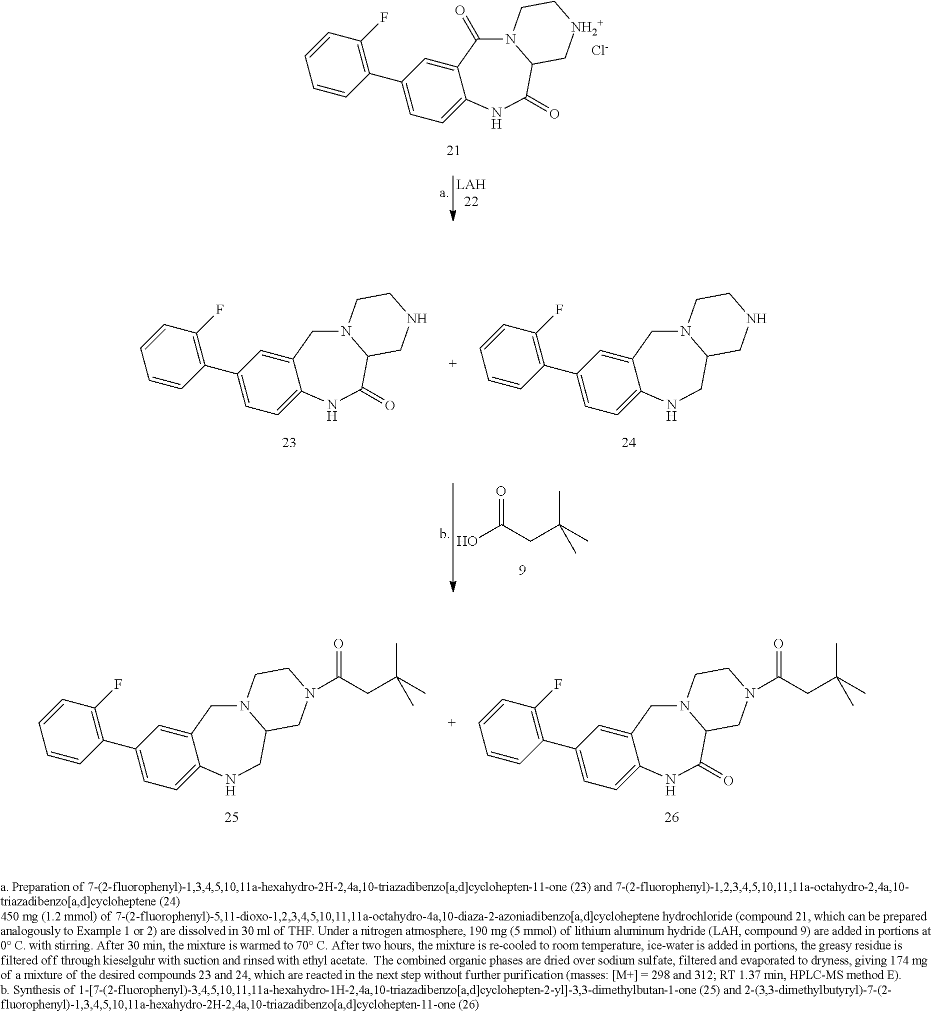

170 mg (about 0.6 mmol) of a mixture of compounds 23 and 24 and 67 mg (0.6 mmol) of dimethylbutyric acid (compound 9) are dissolved in 2 ml of DMF, and 134 mg (0.7 mmol) of DAPECI and 92 mg (0.6 mmol) of HOBT are subsequently added. The mixture is stirred at RT for 3 h. The reaction mixture is subsequently evaporated, and the residue is purified via preparative HPLC (method: 20_{—}40_{—}10).

Combination of the associated fractions and evaporation in vacuo gives the desired products:

77 mg (0.181 mmol, 31% yield) of compound 25 (mass: [M+]=396; RT 2.70 min, HPLC method 1_{—}100_{—}2 Speed).
9.5 mg (0.022 mmol, 4% yield) of compound 26 (mass: [M+]=410; RT 2.95 min, HPLC method 1_{—}100_{—}2 Speed);

¹H NMR (500 MHz, DMSO-d₆) δ [ppm] 10.47 (m, 1H), 7.66-7.50 (m, 3H), 7.43 (m, 1H), 7.36-7.27 (m, 2H), 7.19 (d, J=8.5, 1 H), 4.40-1.93 (m, 11H), 0.95 (d, J=6.6, 9H) [rotamer mixture].

### EXAMPLE 4

### Preparation of 7-(4-chloro-2-fluorophenyl)-2-(3,3-dimethylbutyl)-1,3,4,11a-tetrahydro-2H,10H-2,4-a,10-triazadibenzo[a,d]cycloheptene-5,11-dione (28)

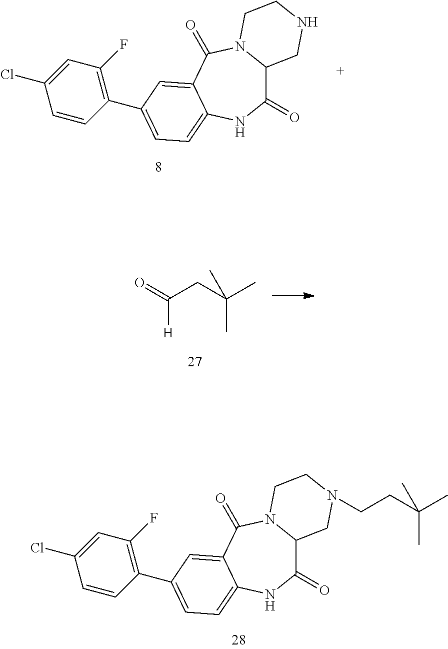

100 mg (0.3 mmol) of compound 8 (prepared analogously to Example 1, a.-e.) and 28 mg (0.3 mmol) of 3,3-dimethylbutyraldehyde (27) are initially introduced in 2 ml of dichloroethane and 1 ml of THF, and 17 mg (0.3 mmol) of acetic acid are added. The solution is then stirred at room temperature for 2 h. 107 mg (0.5 mmol) of sodium triacetoxyborohydride are then added, and the mixture is stirred for a further 14 h. Saturated sodium hydrogencarbonate solution is added to the batch, which is then extracted 2× with ethyl acetate and dried over sodium sulfate and filtered. The filtrate is evaporated to dryness and filtered through a silica gel with ethyl acetate, giving the desired product 28 in a yield of 35% (46 mg, 0.1 mmol), (mass: [M+]=444; RT 2.95 min, HPLC method 1_{—}100_{—}2 Speed).

### EXAMPLE 5

### Preparation of 7-(4-chlorophenyl)-2-(3,3-dimethylbutyryl)-10-methyl-1,3,4,11a-tetrahydro-2H,10H-2,4-a,10-triazadibenzo[a,d]cycloheptene-5,11-dione (“31”)

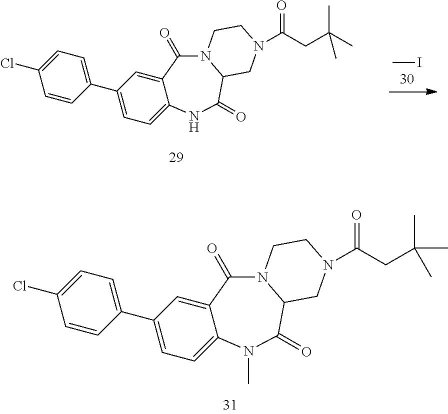

100 mg of compound 29 (0.2 mmol, prepared analogously to Example 1, a. -f.) are dissolved in 10 ml of THF and stirred under a nitrogen atmosphere for a few minutes. The mixture is then cooled to 0° C., and 6.5 mg (0.3 mmol) of sodium hydride (as 60% suspension in paraffin oil) are added. Stirring is continued, and the mixture is warmed to room temperature. After 30 minutes, a clear solution forms. This is re-cooled to 0° C., and 28 mg of methyl iodide are added. The mixture is again warmed to room temperature and stirred for a further 14 h.

The solvent is then removed in vacuo, and the residue is diluted in ethyl acetate. The mixture is washed with water and saturated sodium chloride solution. The mixture is dried over sodium sulfate, filtered, and the solvent is removed. The yellow solid residue which remains (130 mg) is purified via preparative HPLC (method 25_{—}50_{—}10), giving the desired product 31 as white solid (30 mg, 0.06 mmol, 28% yield; mass: [M+]=454; RT 3.42 min, HPLC method 1_{—}100_{—}2);

¹H NMR (500 MHz, DMSO-d₆) δ [ppm] 7.99-7.92 (m, 2H), 7.79-7.73 (m, 2H), 7.57-7.51 (m, 3H), 4.32 (m, 1H), 4.23-4.08 and 3.98-3.89 (2×m, 2H), 3.78-3.66 (m, 2H), 3.61 (m, 1H), 3.45-3.33 (m, 1H), 3.37 (s, 3H), 2.49-2.16 (m, 2H), 1.04 (m, 9H).

### EXAMPLE 6

### Preparation of 7-(4-chlorophenyl)-10-ethyl-2-((S)-2-hydroxy-3,3-dimethyl-butyryl)-1,3,4,11a-tetrahydro-2H,10H-2,4-a,10-triazadibenzo[a,d]cycloheptene-5,11-dione (37)

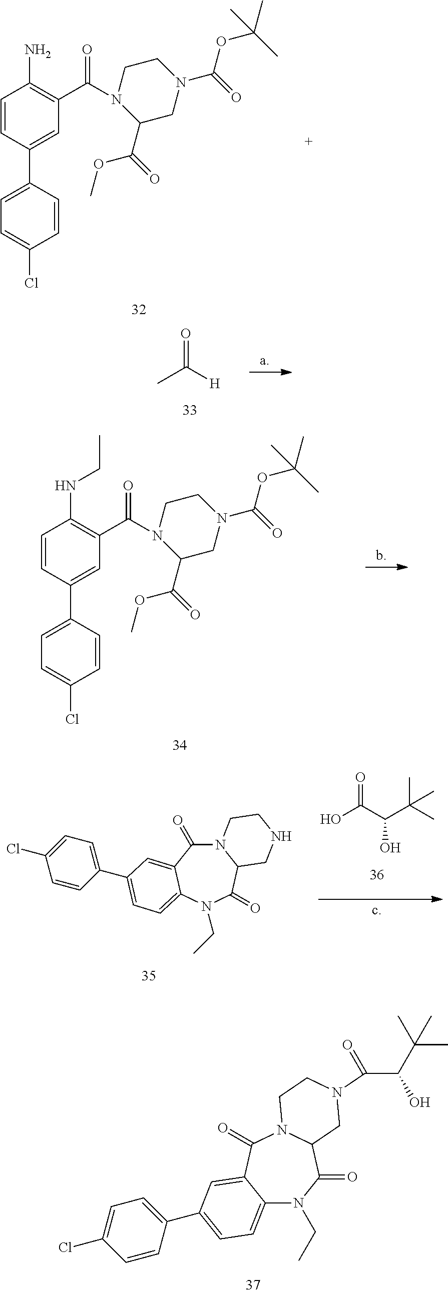

a. Preparation of 1-tert-butyl 3-methyl 4-(4′-chloro-4-ethylaminobiphenyl-3-carbonyl)piperazine-1,3-dicarboxylate (34)

200 mg (0.4 mmol) of compound 32 (prepared analogously to Example 1 a.-d.) are dissolved in 10 ml of dichloroethane and siirred under a nitrogen atmosphere. 18 mg of acetaldehyde (33) and one drop of acetic acid are then added. After 5 minutes, 148 mg (0.7 mmol) of sodium triacetoxyborohydride are added to the yellow reaction mixture, and the reaction is stirred further overnight at room temperature.

The reaction mixture is washed with water and saturated sodium chloride solution, dried over sodium sulfate and filtered. The filtrate is evaporated in vacuo, and the residue is purified via preparative HPLC (method 40_{—}70_{—}10), thus giving the desired product 34 as brown solid (70 mg, 0.13 mmol, 31% yield; mass: [M+ without BOC]=402; RT 4.09 min, HPLC method 1_{—}100_{—}2).

b. Preparation of 7-(4-chlorophenyl)-10-ethyl-1,3,4,11a-tetrahydro-2H, 10H-2,4a,10-triazadibenzo[a,d]cycloheptene-5,11-dione (35)

60 mg of compound 34 (0.1 mmol) and 35 ml of acetic acid in a flask with reflux condenser is stirred at 110° C. for 3 h and then cooled. 25 ml of HCl in methanol are then added at room temperature, and the reaction is stirred for a further 1.25 h.

Water is added, and the pH is adjusted to 9 using 2N NaOH. The mixture is extracted with dichloromethane. The combined organic phases are washed with water and saturated sodium chloride solution, dried over sodium sulfate, filtered and evaporated. The resultant yellow solid 35 (40 mg, 0.094 mmol, 79% yield) is reacted further without further purification. (Mass: [M+]=470; RT 2.57 min, HPLC method 1_{—}1000_{—}2).

c. Preparation of 7-(4-chlorophenyl)-10-ethyl-2-((S)-2-hydroxy-3,3-dimethyl-butyryl)-1,3,4,11a-tetrahydro-2H,10H-2,4a,10-triazadibenzo[a,d]cycloheptene-5,11-dione (37)
Compounds 35 (40 mg, 0.09 mmol), 36 (15 mg, 0.09 mmol) and 4-methyl-morpholine (10 mg, 0.1 mmol) are dissolved in 2 ml of DMF in a flask. 25 mg (0.1 mmol) of EDCI and 20 mg (0.1 mmol) of HOBT are then added, and the reaction mixture is stirred at room temperature for 14 h. The yellow reaction mixture is diluted with ethyl acetate, and water is added. The organic phase is separated off, washed with water and saturated sodium chloride solution, dried over sodium sulfate, filtered and evaporated. The resultant yellow residue (26 mg) is purified via preparative HPLC (method 25_{—}50_{—}10), giving the desired product 37 (9 mg, 0.02 mmol, 17% yield; mass: [M+]=484; RT 3.33-3.36 min, HPLC method 1_{—}100_{—}2).

The following is obtained analogously

### 7-(4-chlorophenyl)-10-ethyl-2-((S)-2-hydroxy-3,3-dimethylbutyryl)-1,3,4,11a-tetrahydro-2H,10H-2,4-a,10-triazadibenzo[a,d]cycloheptene-5,11-dione (“A57”)

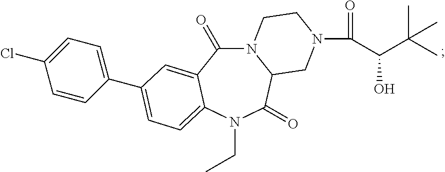

HPLC method D; RT 3.33 min;

¹H NMR (500 MHz, DMSO-d₆) δ [ppm] 10.53 (s, 1H), 7.95 (s, 1H), 7.85 (dd, J=8.4, 2.3, 1H), 7.72 (s, 2H), 7.53 (d, J=8.5, 2H), 7.26 (m, 1H), 4.57-2.73 (m, 13H), 1.58-1.32 (m, 9H).

### EXAMPLE 7

### Preparation of the enantiomers (S)- and (R)-7-(4-chlorophenyl)-1,3,4,11a-tetrahydro-2H,10H-2,4-a,10-triazadibenzo[a,d]cycloheptene-5,11-dione (“19” and “20”)

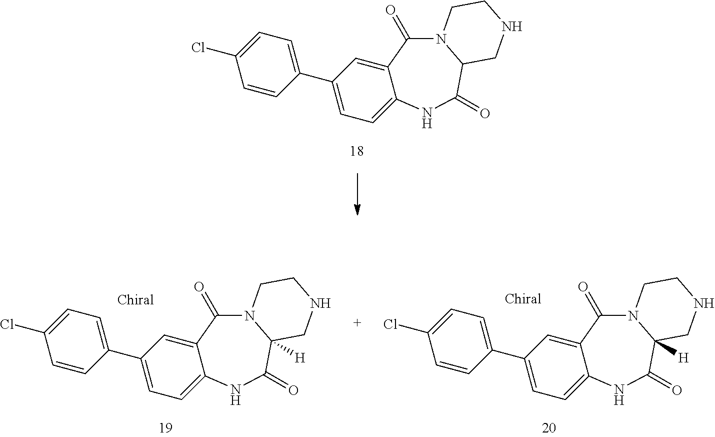

About 145 mg of substance 18 are dissolved in 25 ml of methanol/2 ml of diethylamine/10 ml of acetonitrile in an ultrasound bath and separated on a preparative SFC over a 3×25 cm 5 μm Chiralpak AS-H column with 80 ml of CO₂/20 ml of MeOH+5% of diethylamine.

Combination of identical fractions and evaporation gave the (S)-enantiomer (19, 65 mg, 0.19 mmol, 45%) and the (R)-enantiomer (20, 86 mg, 0.25 mmol), in each case in enantiomerically pure form. These can be reacted further analogously to compound 8.

### EXAMPLE 8

### Preparation of (R)-7-(4-chloro-2-fluorophenyl)-1,3,4,11a-tetrahydro-2H,10H-2,4-a,10-triazadibenzo[a,d]cycloheptene-5,11-dione ((R)-8) and 7-(4-chloro-2-fluorophenyl)-1,3,4,11a-tetrahydro-2H,10H-2,4-a,10-triaza-dibenzo[a,d]cycloheptene-5,11-dione ((S)-8)

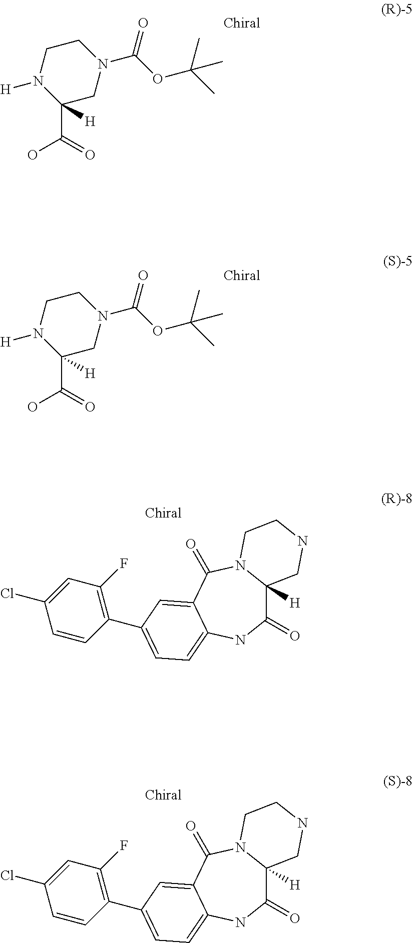

a. Synthesis of (R)-7-(4-chloro-2-fluorophenyl)-1,3,4,11a-tetrahydro-2H,10H-2,4-a,10-triazadibenzo[a,d]cycloheptene-5,11-dione ((R)-8) Analogously to Example 1, a.-e., merely with compound 5 being replaced by enantiomerically pure (R)-5, giving the desired compound (R)-8 in enantiomerically pure form (mass: [M+]=360; RT 2.45 min, HPLC method 1_{—}100_{—}2=method D).

b. Synthesis of (S)-7-(4-chloro-2-fluorophenyl)-1,3,4,11a-tetrahydro-2H,10H-2,4-a,10-triazadibenzo[a,d]cycloheptene-5,11-dione ((S)-8) Analogously to Example 1, a.-e., merely with compound 5 being replaced by enantiomerically pure (S)-5, giving the desired compound (S)-8 in enantiomerically pure form (mass: [M+]=397; RT 2.44 min, HPLC method 1_{—}100_{—}2=method D).

### EXAMPLE 9

### Preparation of benzyl 7-(4-chlorophenyl)-5,11-dioxo-3,4,5,10,11,11a-hexa-hydro-1H-2,4-a,10-triazadibenzo[a,d]cycloheptene-2-carboxylate (40)

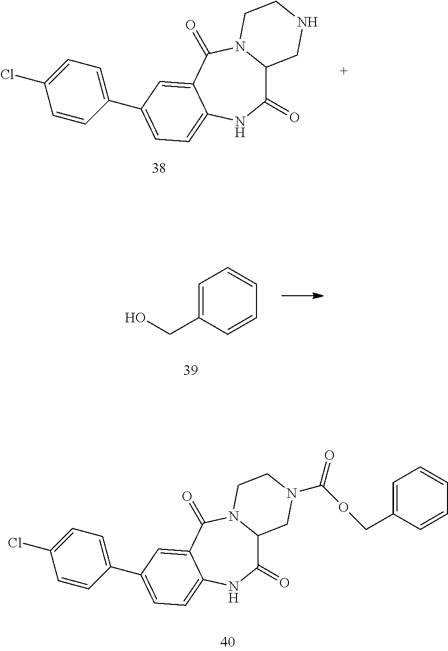

0.1 ml (0.5 mmol) of benzyl alcohol 39 and 79 mg (0.5 mmol) of carbonyl-diimidazole are dissolved in 2 ml of anhydrous DMF and stirred at room temperature for 2 h. A solution of 166 mg (0.5 mmol) of compound 38 in 2 ml of anhydrous DMF is then added, and the resultant solution is stirred at room temperature for 4 days. The yellow reaction solution is then diluted with ethyl acetate and washed twice with water and once with saturated NaCl solution. The organic phase is subsequently dried over Na₂SO₄, filtered and evaporated in vacuo, leaving a yellow powder. This is purified via preparative HPLC (method HPLC 25_{—}50_{—}10), thus giving the desired product 40 as yellow solid (19 mg, 0.04 mmol, 8% yield; mass: [M+]=476; RT 3.46 min, HPLC method 1_{—}100_{—}2=method D).

### EXAMPLE 10

### Preparation of (R)-7-(4-chloro-2-fluorophenyl)-2-(imidazole-1-carbonyl)-1,3,4,11a-tetrahydro-2H,10H-2,4-a,10-triazadibenzo[a,d]cycloheptene-5,11-dione (41)

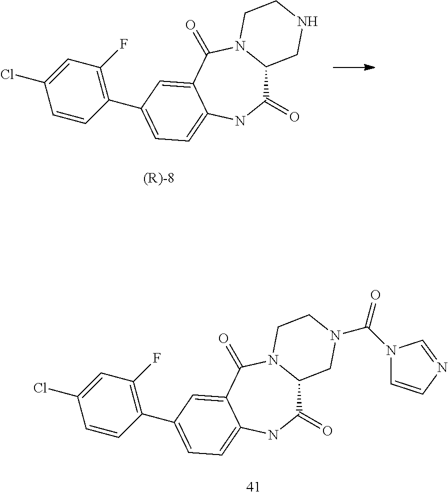

199 mg (0.5 mmol) of compound (R)-8 are dissolved in 10 ml of dichloro-methane, and 81 mg (0.5 mmol) of carbonyldiimidazole and 69 μl (0.5 mmol) of triethylamine are added. The mixture is stirred overnight at room temperature. The solution is then evaporated to dryness, and the residue is purified via preparative HPLC (method 25_{—}50_{—}10), thus giving the desired product 41 as yellowish solid (61 mg, yield 27%, mass: [M+]=454; RT 2.86 min, HPLC method 1_{—}100_{—}2=method D).

### EXAMPLE 11

### Preparation of 4′-chloro-4-({(R)-4-[(S)-3,3-dimethyl-2-(piperidin-4-ylamino)-butyryl]piperazine-2-carbonyl}amino)-2′-fluorobiphenyl-3-carboxylic acid (47) and (R)-7-(4-chloro-2-fluorophenyl)-2-[(S)-3,3-dimethyl-2-(piperidin-4-ylamino)butyryl]-1,3,4,11a-tetrahydro-2H,10H-2,4-a,10-triazadibenzo[a,d]-cycloheptene-5,11-dione (48)

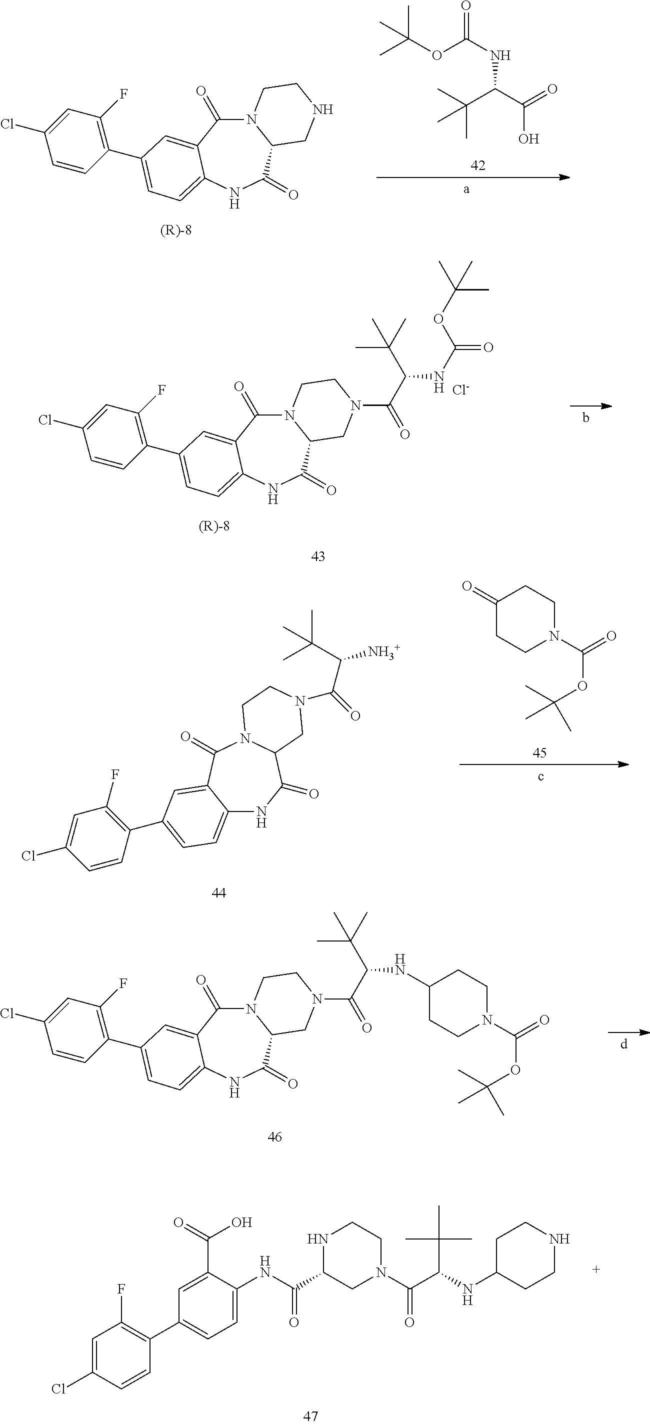

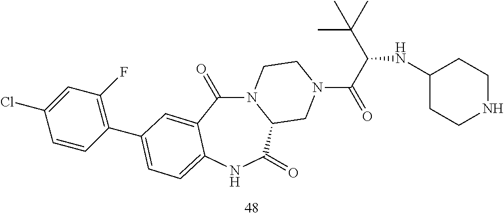

a. Synthesis of tert-butyl {(S)-1-[(R)-7-(4-chloro-2-fluorophenyl)-5,11-dioxo-3,4,5,10,11,11a-hexahydro-1H-2,4a,10-triazadibenzo[a,d]cycloheptene-2-carbonyl]-2,2-dimethylpropyl}carbamate (43)

Starting material (R)-8 (198 mg, 0.5 mmol) is initially introduced in 10 ml of dichloromethane, DIPEA (0.1 ml, 0.5 mmol) and subsequently 96 mg (0.5 mmol) of DAPECI and 76 mg (0.5 mmol) of HOBT hydrate and starting material 42 (116 mg, 0.5 mmol) are added to the resultant solution, and the reaction mixture is stirred at room temperature. After 8 h, the organic reaction mixture is washed twice with water and dried over sodium sulfate, filtered, and the filtrate is evaporated to dryness (R1), thus giving the desired product 43 (260 mg, 0.45 mmol, 91% yield, mass: [M+ without Boc]=473; HPLC method D, RT=3.55 min) as solid, which is reacted further without further purification.

b. Synthesis of (S)-1-[(R)-7-(4-chloro-2-fluorophenyl)-5,11-dioxo- 3,4,5,10,11,11a-hexahydro-1H-2,4a,10-triazadibenzo[a,d]cycloheptene-2- carbonyl]-2,2dimethylpropylammonium (44)

260 mg (0.5 mmol) of compound 43 are dissolved in 10 ml of 4N HCL in dioxane and stirred at room temperature for 2 h. The reaction mixture is subsequently evaporated to dryness, and the solid residue is triturated with acetonitrile. The resultant crystals are filtered off with suction and rinsed with a little acetonitrile, thus isolating the desired product 44 (105 mg, 0.2 mmol, 45% yield, HPLC method D, RT=2.57 min), which is reacted in the next step without further purification.

c. Synthesis of tert-butyl 4-{(S)-1-[(R)-7-(4-chloro-2-fluorophenyl)-5,11- dioxo-3,4,5,10,11,11a-hexahydro-1H-2,4a,10-triazadibenzo[a,d]cyclo- heptene-2-carbonyl]-2,2-dimethylpropylamino}piperidine-1-carboxylate (46)

Compound 44 (105 mg, 0.2 mmol) and 1-Boc-4-piperidone 45 (50 mg, 0.3 mmol) are dissolved in a mixture of 10 ml of 1,2-dichloroethane and 2 ml of dioxane and stirred at room temperature for 2 h. 108 mg (0.5 mmol) of sodium triacetoxyborohydride are then added in 2 portions, and the mixture is left to stir at RT for a turther 4 h. The reaction solution is evaporated to dryness, and the residue is purified via the preparative HPLC apparatus (method 5_{—}70_{—}10). Combination and evaporation of the corresponding fractions isolates the desired product 46 (73 mg, 0.11 mmol, 44% yield, mass: [M+]=656; HPLC method D, RT=2.83 min) as colourless solid.

d. Synthesis of 4,′-chloro-4-({(R)-4-[(S)-3,3-dimethyl-2-(piperidin-4-yl- amino)butyryl]piperazine-2-carbonyl}amino)-2′-fluorobiphenyl-3-carboxylic acid (47) and (R)-7-(4-chloro-2-fluorophenyl)-2-[(S)-3,3-dimethyl-2-(piperidin-4-ylamino)butyryl]-1,3,4,11a-tetrahydro-2H,10H-2,4a,10-triazadibenzo-[a,d]cycloheptene-5,11-dione (48)

73 mg (0.1 mmol) of compound 46 are dissolved in 30 ml of 2N HCL and stirred at room temperature for 2 h. The reaction mixture is subsequently evaporated to dryness, and the solid residue is triturated with acetonitrile. The resultant crystals are filtered off with suction and rinsed with a little acetonitrile. The crystals are purified again via the preparative HPLC apparatus (method 1_{—}60_{—}10), thus isolating the products 47 (10.5 mg, 0.018 mmol, 16% yield, mass: [M+]=574; HPLC method D, RT=2.55 min) and 48 (24.8 mg, 0.045 mmol, 40% yield, mass: [M+]=556; HPLC method D, RT=2.40 min).

### EXAMPLE 12

### Preparation of (R)-7-(4-chloro-2-fluorophenyl)-2-phenylmethanesulfonyl-1,3,4,11a-tetrahydro-2H,10H-2,4-a,10-triazadibenzo[a,d]cycloheptene-5,11-dione (50)

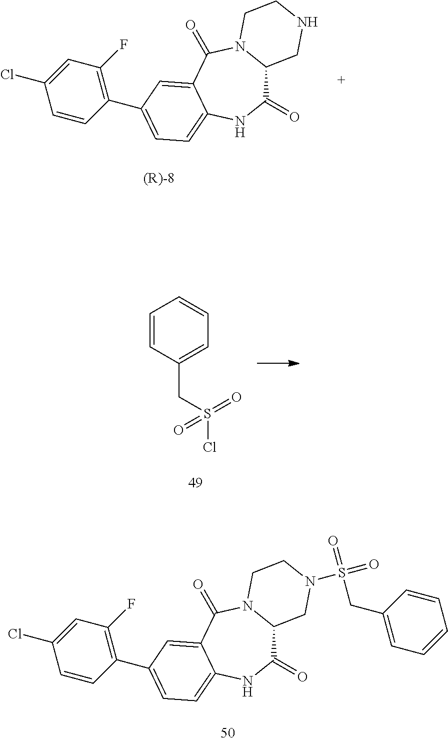

100 mg (0.3 mmol) of (R)-8 are dissolved in 5 ml of dichloromethane. 81 mg (0.4 mmol) of phenylmethanesulfonyl chloride and 80 μl of triethylamine (0.6 mmol) are subsequently added. The mixture is stirred at room temperature for 2.5 h. The reaction solution is then washed with dilute HCl and water, dried over sodium sulfate and evaporated to dryness. The residue is purified via preparative HPLC (method 25_{—}50_{—}10_{—}50ml_empfind_o_equi.M), thus giving 8 mg (0.016 mmol, 6% yield) of the desired product 50 (mass: [M+]=513; HPLC method D, RT=3.46 min);

¹H NMR (400 MHz, DMSO-d₆) δ [ppm] 10.71 (s, 1H), 7.98-7.88 (m, 1H), 7.78-7.71 (m, 1H), 7.62 (t, J=8.6, 1 H), 7.57 (dd, J=10.8, 2.1, 1 H), 7.42 (dd, J=7.4, 1.8, 3H), 7.36-7.30 (m, 3H), 7.25 (d, J=8.5, 1 H), 4.53 (q, J=13.7, 2H), 4.32-4.19 (m, 2H), 3.96 (dd, J=13.7, 5.0, 1H), 3.51-3.22 (m, 2H), 3.22-3.11 (m, 2H).

### EXAMPLE 13

### Preparation of 7-(4-chlorophenyl)-2-((S)-2-hydroxy-3,3-dimethylbutyryl)-1,3,4,5,10,11a-hexahydro-2H-2,4-a,10-triazadibenzo[a,d]cyclohepten-11-one (57)

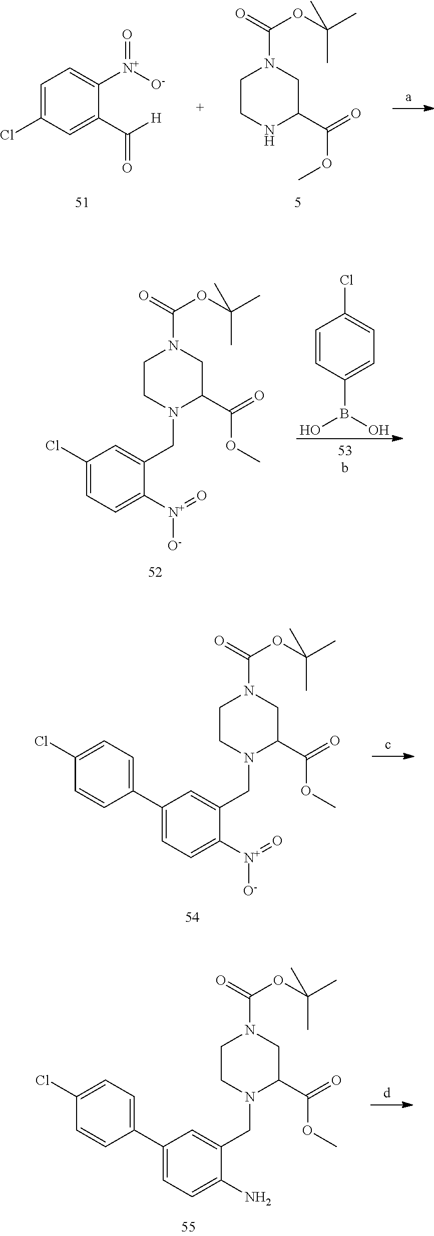

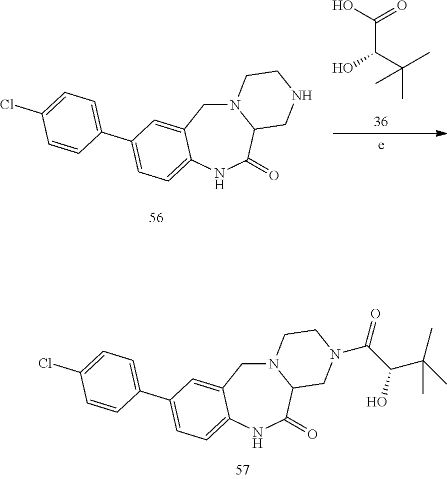

a. Preparation of 1-tert-butyl 3-methyl 4-(5-chloro-2-nitrobenzyl)piperazine-1,3-dicarboxylate (52)

1.5 g (8.2 mmol) of the aldehyde 51 and 2.0 g (8.2 mmol) of the amine 5 are initially introduced in a mixture of 50 ml of dichloroethane and 50 ml of THF. 0.940 ml of glacial acetic acid are then added, and the mixture is stirred at RT for about 3 h. 5.5 g (24.6 mmol) of NaB(OAc)₃ and a further 0.940 ml of acetic acid are subsequently added, and the mixture is stirred overnight at room temperature. The batch is stirred with saturated NaHCO₃ solution, diluted with dichloromethane and extracted by shaking. The organic phase is again washed by shaking with water, the aqueous phase is again extracted by shaking with DCM. The combined organic phases are dried over Na₂SO₄, filtered off with suction and evaporated to dryness in vacuo. The 3.5 g of crude product obtained are dissolved in THF, adsorbed onto Isolute and separated on silica gel 60 (Flashmaster). The relevant fractions are combined and evaporated to dryness in a rotary evaporator, thus giving the desired product 52 (1.6 g, 21% yield) in a purity of 45% (mass: [M+]=414; HPLC method D, RT=3.86 min) as yellow oil, which is reacted further without further purification.

b. Preparation of 1-tert-butyl 3-methyl 4-(4′-chloro-4-nitrobiphenyl-3-yl-methyl)piperazine-1,3-dicarboxylate (54)

Starting materials 52 (1.6 g, 3.9 mmol), 53 (605 mg, 3.9 mmol), 10 ml of ethylene glycol dimethyl ether and 5 ml of water are initially introduced in a microwave vial (10-20 ml) (clear solution), and 1.1 g (13.5 mmol) of sodium hydrogencarbonate are then added in portions with stirring (suspension). Nitrogen is introduced into the suspension by means of a cannula with stirring. 447 mg (0.3 mmol) of tetrakistriphenylphosphinepalladium are added in a counter-stream of nitrogen, and the MW vessel is sealed. The reaction mixture is heated by means of the microwave (140° C., 30 min). The batch is filtered and rinsed with EA. The filtrate is diluted with EA and water and washed by shaking. The organic phase is again washed with water. The organic phase is washed with saturated NaCl solution, dried over Na₂SO₄, filtered off with suction and evaporated to dryness in vacuo. This organic residue (2.3 g, 19% according 10 HPLC, black viscous mass) is dissolved in DCM, adsorbed onto Isolute adsorbent and purified on silica gel 60 (Companion). The relevant fractions are combined and evaporated to dryness in vacuo, thus giving the desired product 54 in a purity of about 50% (1.3 g, mass: [M+]=490; HPLC method D, RT=4.27 min) as yellow, viscous mass, which is reacted further without further purification.

c. Preparation of 1-tert-butyl 3-methyl 4-(4-amino-4′-chlorobiphenyl-3-yl- methyl)piperazine-1,3-dicarboxylate (55)

1.2 g (1.2 mmol) of the starting material 54 are hydrogenated using hydrogen on a sponge nickel catalyst (1 g) in 20 ml of THF at room temperature for 22 h. 82 ml of hydrogen are taken up in the process. Evaporation of the reaction solution in vacuo gives the desired product 55 as brown, viscous mass (1.1 g, 62% purity, mass: [M+]=460; HPLC method D, RT=4.03 min).

d. Preparation ot 7-(4-chlorophenyl)-1,3,4,5,10,11a-hexahydro-2H-2,4a,10- triazadibenzo[a,d]cyclohepten-11-one (56)

Starting material 55 (1.1 g, 1.5 mmol) and 15 ml of acetic acid are introduced into a 50 ml round-bottomed flask and stirred at 110° C. for 2 h (yellow solution). 10 ml of HCl/isopropanol (5-6 N) are then added in order to cleave off the BOC group. The mixture is stirred for a further hour, and the yellow solution is then evaporated to dryness. This residue is stirred with water and EA. Saturated NaHCO₃ solution is then added until pH 8 becomes established. The phases are then separated. The aqueous phase is extracted by shaking a further twice with n-butanol. The combined organic phases are dried over Na₂SO₄, filtered off with suction and evaporated to dryness in vacuo, giving the desired product 56 (790 mg, 25% yield, mass: [M+]=328; HPLC method D, RT=2.45 min) as brown solid.

e. Preparation of 7-(4-chlorophenyl)-2-((S)-2-hydroxy-3,3-dimethylbutyryl)- 1,3,4,5,10,11a-hexahydro-2H-2,4a,10-triazadibenzo[a,d]cyclohepten-11-one (57)

250 mg (0.8 mmol) of the starting material 56, 101 mg (0.8 mmol) of the starting material 5, 219 mg (1.1 mmol) of DIPEA, 152 mg (1.0 mmol) of HOBt hydrate and 5 ml of DMF are introduced successively, together with a magnetic stirrer bar, into a reaction vessel (2-5 ml), which is then sealed by means of a septum and heated by means of microwaves (120° C., 30 min). The batch is diluted with ethyl acetate and water and washed by shaking. The organic phase is washed again with water and saturated NaCl solution. The organic phase is dried over Na₂SO₄, filtered off with suction and evaporated to dryness in vacuo. The residue obtained is dissolved in DMSO and purified on the preparative HPLC on RP silica gel (method 15_{—}35_{—}10_{—}50 ml_normal_o_- equi.M). The relevant fractions are combined and evaporated to dryness in vacuo. After evaporation (110 mg), the contaminated fractions are purified again on RP silica gel (prep. HPLC. method 15_{—}35_{—}10_{—}50 ml_normal_o_- equi.M). The relevant fractions are combined, the acetonitrile is distilled off in vacuo, and the aqueous residue is freeze-dried. The desired product 57 is isolated as colourless solid (39.5 mg, 0.09 mmol, 12% yield, mass: [M+]=442; HPLC method D, RT=3.16 min).

### EXAMPLE 14

The following compounds are obtained analogously to Example 1

| | | HPLC |
|---|---|---|
| Compound | | method; |
| No. | Name and/or structure | RT [min] |
| “A58” | 7-(4-Chloro-2-fluorophenyl)-2-(2-methyl-2-phenyl- propionyl)-1,3,4,11a-tetrahydro-2H,10H-2,4a,10- triazadibenzo[a,d]cycloheptene-5,11-dione | D; 3.44 |
| | 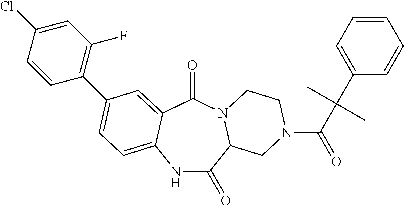 | |
| “A59” | 2-((S)-2-Amino-3-methylbutyryl)-7-(4-chloro-2- fluorophenyl)-1,3,4,11a-tetrahydro-2H,10H-2,4a,10- triazadibenzo[a,d]cycloheptene-5,11-dione | D; 2.49 |
| | 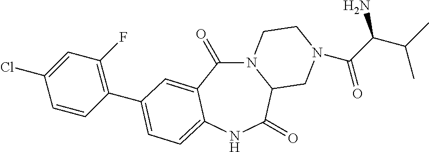 | |
| “A60” | 7-(4-Chloro-2-fluorophenyl)-2-((S)-2-hydroxy-3,3- dimethylbutyryl)-1,3,4,11a-tetrahydro-2H,10H- 2,4a,10-triazadibenzo[a,d]cycloheptene-5,11-dione | D; 3.17 |
| | 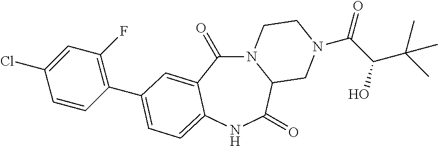 | |
| “A61” | 2-((S)-2-Amino-3,3-dimethylbutyryl)-7-(4-chloro-2- fluorophenyl)-1,3,4,11a-tetrahydro-2H,10H-2,4a,10- triazadibenzo[a,d]cycloheptene-5,11-dione | D; 2.59 |
| | 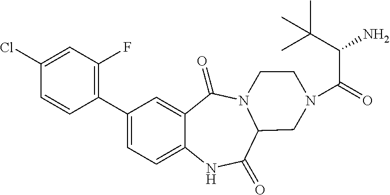 | |
| “A62” | (S)-2-((S)-2-Amino-3-methylbutyryl)-7-(4-chloro-2- fluorophenyl)-1,3,4,11a-tetrahydro-2H,10H-2,4a,10- triazadibenzo[a,d]cycloheptene-5,11-dione | D; 2.49 |
| | 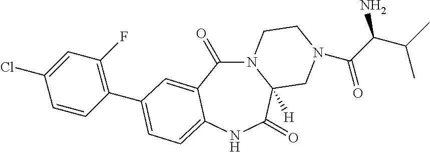 | |
| “A63” | (R)-2-((S)-2-Amino-3-methylbutyryl)-7-(4-chloro-2- fluorophenyl)-1,3,4,11a-tetrahydro-2H,10H-2,4a,10- triazadibenzo[a,d]cycloheptene-5,11-dione | D; 2.49 |
| | 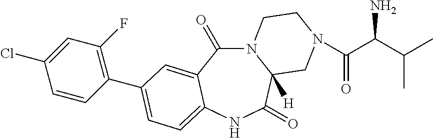 | |
| “A64” | (S)-7-(4-Chloro-2-fluorophenyl)-2-((S)-2-hydroxy-3,3- dimethylbutyryl)-1,3,4,11a-tetrahydro-2H,10H- 2,4a,10-triazadibenzo[a,d]cycloheptene-5,11-dione | D; 3.17 |
| | 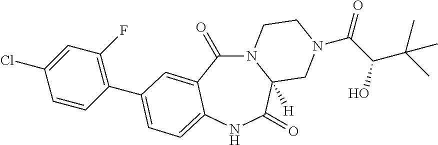 | |
| “A65” | (R)-2-((S)-2-Amino-3,3-dimethylbutyryl)-7-(4-chloro- 2-fluorophenyl)-1,3,4,11a-tetrahydro-2H,10H- 2,4a,10-triazadibenzo[a,d]cycloheptene-5,11-dione | D; 2.59 |
| | 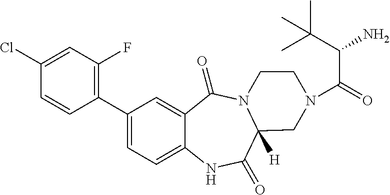 | |
| “A66” | (S)-2-((S)-2-Amino-3,3-dimethylbutyryl)-7-(4-chloro- 2-fluorophenyl)-1,3,4,11a-tetrahydro-2H,10H- 2,4a,10-triazadibenzo[a,d]cycloheptene-5,11-dione | D; 2.59 |
| | 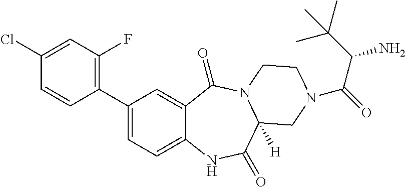 | |
| “A67” | 7-(4-Chloro-2-fluorophenyl)-2-(3-methyl-3H- imidazole-4-carbonyl)-1,3,4,11a-tetrahydro-2H,10H- 2,4a,10-triazadibenzo[a,d]cycloheptene-5,11-dione | D; 2.54 |
| | 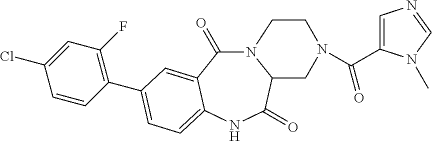 | |
| “A68” | 7-(4-Chloro-2-fluorophenyl)-2-((R)-5,5-dimethyl- thiazolidine-4-carbonyl)-1,3,4,11a-tetrahydro- 2H,10H-2,4a,10-triazadibenzo[a,d]cycloheptene- 5,11-dione | D; 2.73 |
| | 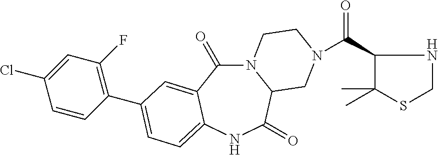 | |
| “A69” | 7-(4-Chloro-2-fluorophenyl)-2-((2S,3S)-3-hydroxy- pyrrolidine-2-carbonyl)-1,3,4,11a-tetrahydro-2H,10H- 2,4a,10-triazadibenzo[a,d]cycloheptene-5,11-dione | D; 2.47 |
| | 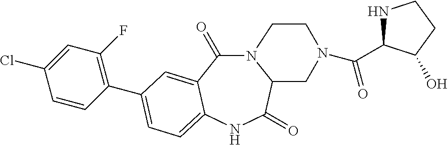 | |
| “A70” | 7-(4-Chloro-2-fluorophenyl)-2-((2S,4R)-4-hydroxy- pyrrolidine-2-carbonyl)-1,3,4,11a-tetrahydro-2H,10H- 2,4a,10-triazadibenzo[a,d]cycloheptene-5,11-dione | D; 2.47 |
| | 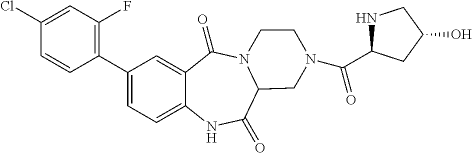 | |
| “A71” | 7-(4-Chlorophenyl)-2-(2-pyridin-4-ylthiazolidine-4- carbonyl)-1,3,4,11a-tetrahydro-2H,10H-2,4a,10- triazadibenzo[a,d]cycloheptene-5,11-dione | D; 2.48 |
| | 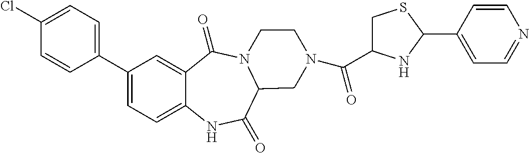 | |
| “A72” | 7-(4-Chlorophenyl)-2-(2-trifluoromethylimidazo[1,2- a]pyridine-3-carbonyl)-1,3,4,11a-tetrahydro-2H,10H- 2,4a,10-triazadibenzo[a,d]cycloheptene-5,11-dione | D; 3.25 |
| | 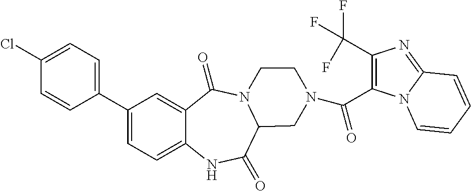 | |
| “A73” | 7-(4-Chlorophenyl)-2-(2,4-dimethylthiazole-5- carbonyl)-1,3,4,11a-tetrahydro-2H,10H-2,4a,10- triazadibenzo[a,d]cycloheptene-5,11-dione | D; 3.0 |
| | 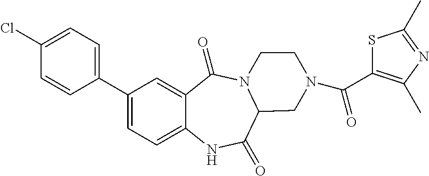 | |
| “A74” | 7-(4-Chlorophenyl)-2-((S)-2-hydroxy-3-methyl- butyryl)-1,3,4,11a-tetrahydro-2H,10H-2,4a,10-triaza- dibenzo[a,d]cycloheptene-5,11-dione | D; 3.01 |
| | 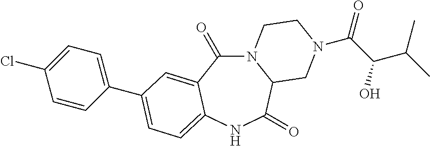 | |
| “A75” | 7-(4-Chlorophenyl)-2-(3H-imidazole-4-carbonyl)- 1,3,4,11a-tetrahydro-2H,10H-2,4a,10-triaza- dibenzo[a,d]cycloheptene-5,11-dione | D; 2.51 |
| | 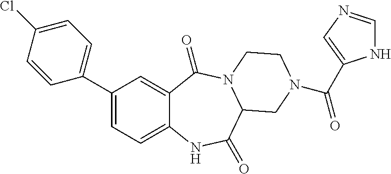 | |
| “A76” | 7-(4-Chlorophenyl)-2-(2-pyridin-3-ylthiazole-4- carbonyl)-1,3,4,11a-tetrahydro-2H,10H-2,4a,10- triazadibenzo[a,d]cycloheptene-5,11-dione | D; 2.97 |
| | 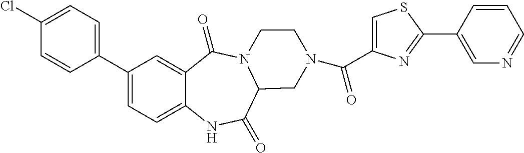 | |
| “A77” | 7-(4-Chloro-2-fluorophenyl)-2-((R)-2-hydroxy-3- methylbutyryl)-1,3,4,11a-tetrahydro-2H,10H-2,4a,10- triazadibenzo[a,d]cycloheptene-5,11-dione | D; 3.51 |
| | 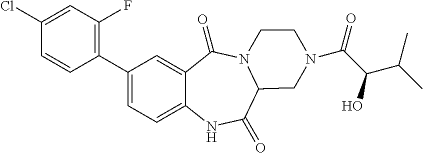 | |
| “A78” | 7-(2,4-Dichlorophenyl)-2-(3,3-dimethylbutyryl)- 1,3,4,11a-tetrahydro-2H,10H-2,4a,10-triaza- dibenzo[a,d]cycloheptene-5,11-dione | D; 3.43 |
| | 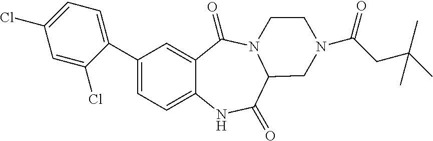 | |

¹H NMR (500 MHz, DMSO-d₆) δ [ppm] 10.68 and 10.62 (2 x s, 1H), 7.84- 7.80 (m, 1H), 7.77 (d, J = 2.1, 1H), 7.64 (ddd, J = 8.3, 3.7, 2.3, 1H), 7.57- 7.52 (m, 1H), 7.48 (dd, J = 8.3, 4.8, 1H), 7.24 (d, J = 8.4, 1H), 4.32 (2 x t, 4.8, 1H), 4.24-4.04 and 3.96-3.87 (2 x m, 2H), 3.84-3.74 and 3.67 (2 x m, 2H), 3.61-3.51 and 3.50-3.41 (2 x m, 2H), 2.36 (q, J = 14.9) and 2.21 (q, J = 14.5) together 2 H, 1.02 and 1.01 (2 x s, 9 H); (rotamer mixture)

| | | |
|---|---|---|
| “A79” | 7-(4-Chloro-3,5-difluorophenyl)-2-(3,3-dimethyl- butyryl)-1,3,4,11a-tetrahydro-2H,10H-2,4a,10- triazadibenzo[a,d]cycloheptene-5,11-dione | D; 3.42 |

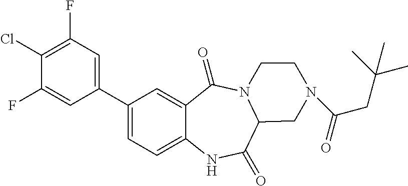

| | | |
|---|---|---|
| “A80” | (S)-2-((S)-2-Amino-3,3-dimethylbutyryl)-7-(4-chloro- phenyl)-1,3,4,11a-tetrahydro-2H,10H-2,4a,10-triaza- dibenzo[a,d]cycloheptene-5,11-dione | D; 2.58 |
| | 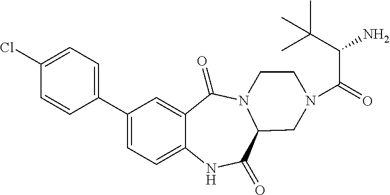 | |
| “A81” | (S)-7-(4-Chlorophenyl)-2-((S)-1-piperidin-4-yl- pyrrolidine-2-carbonyl)-1,3,4,11a-tetrahydro-2H,10H- 2,4a,10-triazadibenzo[a,d]cycloheptene-5,11-dione | D; 2.25 |
| | 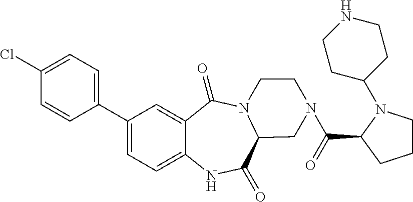 | |
| “A82” | 7-(2,3-Difluoro-4-methylphenyl)-2-(3,3-dimethyl- butyryl)-1,3,4,11a-tetrahydro-2H,10H-2,4a,10- triazadibenzo[a,d]cycloheptene-5,11-dione | D; 3.29 |
| | 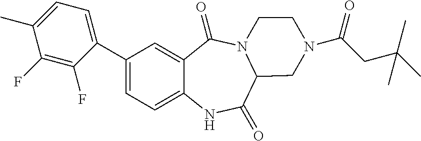 | |
| “A83” | 2-(3,3-Dimethylbutyryl)-7-(2-fluoro-5-trifluoromethyl- phenyl)-1,3,4,11a-tetrahydro-2H,10H-2,4a,10- triazadibenzo[a,d]cycloheptene-5,11-dione | D; 3.34 |
| | 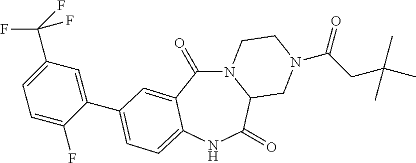 | |
| “A84” | 2-(3,3-Dimethylbutyryl)-7-(2-fluoro-4-trifluoromethyl- phenyl)-1,3,4,11a-tetrahydro-2H,10H-2,4a,10- triazadibenzo[a,d]cycloheptene-5,11-dione | D; 3.4 |
| | 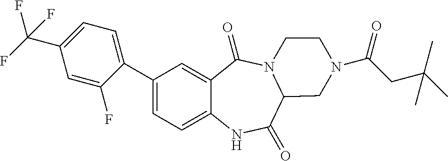 | |
| “A85” | 7-(5-Chloro-2-fluorophenyl)-2-(3,3-dimethylbutyryl)- 1,3,4,11a-tetrahydro-2H,10H-2,4a,10-triaza- dibenzo[a,d]cycloheptene-5,11-dione | D; 3.27 |
| | 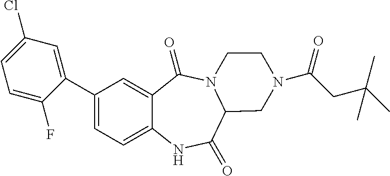 | |
| “A86” | 2-(3,3-Dimethylbutyryl)-7-(2,4-dimethylphenyl)- 1,3,4,11a-tetrahydro-2H,10H-2,4a,10-triaza- dibenzo[a,d]cycloheptene-5,11-dione | D; 3.32 |
| | 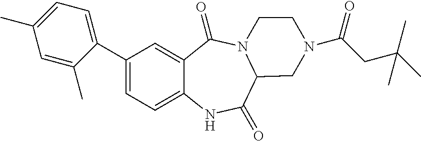 | |
| “A87” | 2-(3,3-Dimethylbutyryl)-7-(3-trifluoromethylphenyl)- 1,3,4,11a-tetrahydro-2H,10H-2,4a,10-triaza- dibenzo[a,d]cycloheptene-5,11-dione | D; 3.31 |
| | 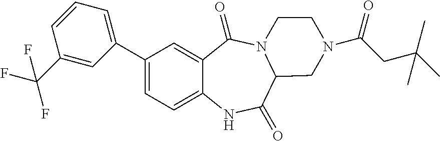 | |
| “A88” | 7-(2,3-Difluorophenyl)-2-(3,3-dimethylbutyryl)- 1,3,4,11a-tetrahydro-2H,10H-2,4a,10-triaza- dibenzo[a,d]cycloheptene-5,11-dione | D; 3.13 |
| | 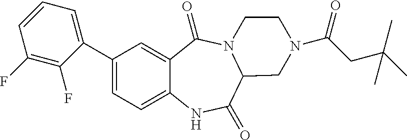 | |
| “A89” | 7-(2,5-Difluorophenyl)-2-(3,3-dimethylbutyryl)- 1,3,4,11a-tetrahydro-2H,10H-2,4a,10-triaza- dibenzo[a,d]cycloheptene-5,11-dione | D; 3.11 |
| | 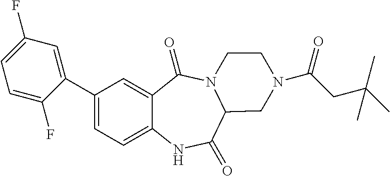 | |
| “A90” | 2-(3,3-Dimethylbutyryl)-7-(3,4-dimethylphenyl)- 1,3,4,11a-tetrahydro-2H,10H-2,4a,10-triaza- dibenzo[a,d]cycloheptene-5,11-dione | D; 3.33 |
| | 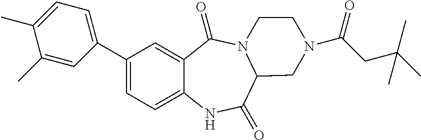 | |
| “A91” | 7-(2,4-Difluorophenyl)-2-(3,3-dimethylbutyryl)- 1,3,4,11a-tetrahydro-2H,10H-2,4a,10-triaza- dibenzo[a,d]cycloheptene-5,11-dione | D; 3.15 |
| | 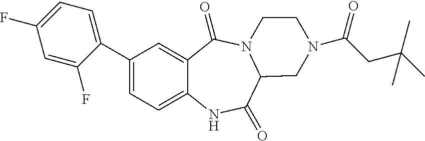 | |
| “A92” | 2-(3,3-Dimethylbutyryl)-7-(2,4,5-trifluorophenyl)- 1,3,4,11a-tetrahydro-2H,10H-2,4a,10-triaza- dibenzo[a,d]cycloheptene-5,11-dione | D; 3.19 |
| | 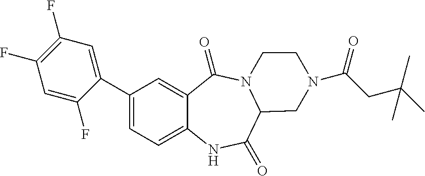 | |
| “A93” | 7-(4-Chloro-2-fluorophenyl)-2-((R)-1-piperidin-4- ylpyrrolidine-2-carbonyl)-1,3,4,11a-tetrahydro- 2H,10H-2,4a,10-triazadibenzo[a,d]cycloheptene- 5,11-dione | D; 2.25 |
| | 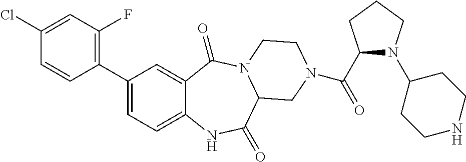 | |
| “A94” | 7-(4-Chloro-3-fluorophenyl)-2-(3,3-dimethylbutyryl)- 1,3,4,11a-tetrahydro-2H,10H-2,4a,10-triaza- dibenzo[a,d]cycloheptene-5,11-dione | D; 3.29 |
| | 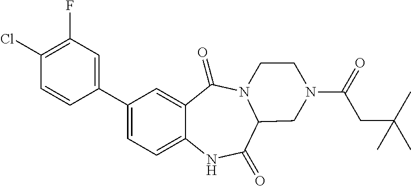 | |
| “A95” | 7-(4-Chloro-2-fluorophenyl)-2-[3-(1-methyl-1H- pyrazol-4-yl)propionyl]-1,3,4,11a-tetrahydro-2H,10H- 2,4a,10-triazadibenzo[a,d]cycloheptene-5,11-dione | D; 2.92 |
| | 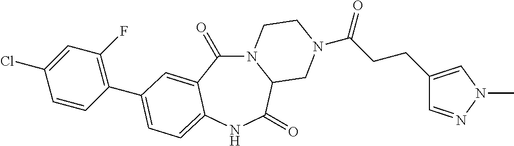 | |
| “A96” | (R)-7-(4-Chloro-2-fluorophenyl)-2-((S)-2-hydroxy-3,3- dimethylbutyryl)-1,3,4,11a-tetrahydro-2H,10H- 2,4a,10-triazadibenzo[a,d]cycloheptene-5,11-dione | D; 3.16 |
| | 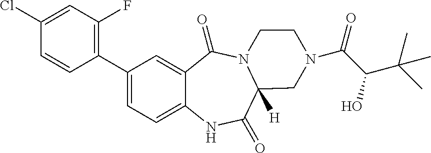 | |

¹H NMR (500 MHz, DMSO-d₆) δ [ppm] 10.70 and 10.60 (2 x s, 1H), 7.93 (d, J = 6.3, 1H), 7.74 (d, J = 8.4, 1H), 7.65-7.54 (m, 2H), 7.41 (dd, J = 8.3, 1.8, 1H), 7.25 (dd, J = 8.4, 5.4, 1H), 4.58 (dd, J = 28.6, 7.6, 1H), 4.44- 3.74 (m, 5H), 3.65-3.41 (m, 3H), 0.92 (d, J = 16.9, 9H)

| | | |
|---|---|---|
| “A97” | (R)-7-(4-Chloro-2-fluorophenyl)-2-[3-(1-methyl-1H- pyrazol-4-yl)propionyl]-1,3,4,11a-tetrahydro-2H,10H- 2,4a,10-triazadibenzo[a,d]cycloheptene-5,11-dione | F; 5.25 |
| | 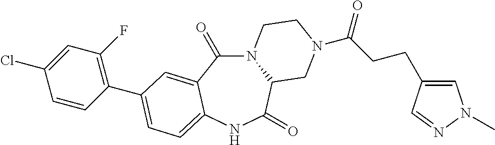 | |
| “A98” | (S)-7-(4-Chloro-2-fIuorophenyl)-2-[3-(1-methyl-1H- pyrazol-4-yl)propionyl]-1,3,4,11a-tetrahydro-2H,10H- 2,4a,10-triazadibenzo[a,d]cycloheptene-5,11-dione | F; 9.23 |
| | 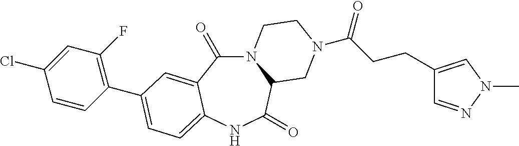 | |
| “A99” | 7-(4-Chloro-2-fluorophenyl)-2-((S)-1-piperidin-4-yl- pyrrolidine-2-carbonyl)-1,3,4,11a-tetrahydro-2H,10H- 2,4a,10-triazadibenzo[a,d]cycloheptene-5,11-dione | D; 2.31 |
| | 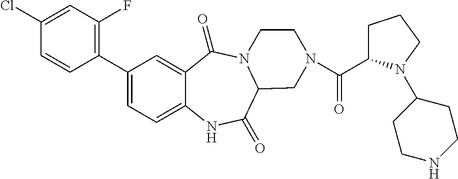 | |

¹H NMR (500 MHz, DMSO-d₆) δ [ppm] 10.82 (d, J = 13.9) and 10.67 (d, J = 6.5) together 1 H, 9.67-9.39 (br. m, 1H), 8.02-7.90 (m, 1H), 7.75 (d, J = 6.3, 1H), 7.64-7.54 (m, 2H), 7.42 (d, J = 8.3, 1H), 7.30-7.22 (m, 1H), 4.86-4.59 (m, 1H), 4.52-4.36 (m, 2H), 4.16-3.88 (m, 4H), 3.85-3.56 (m, 4H), 3.54-3.29 (m, 3H), 3.20 (s, 1H), 3.02-2.72 (m, 2H), 2.39-1.58 (m, 6H).

| | | |
|---|---|---|
| “A100” | (R)-7-(4-Chloro-2-fluorophenyl)-2-(3,3-dimethyl- butyryl)-1,3,4,11a-tetrahydro-2H,10H-2,4a,10- triazadibenzo[a,d]cycloheptene-5,11-dione | D; 3.35 |
| | 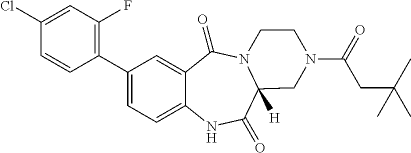 | |

¹H NMR (400 MHz, DMSO-d₆) δ [ppm] 10.61 and 10.56 (2 x s, 1H), 7.86 (d, J = 1.4, 1H), 7.70-7.63 (m, 1H), 7.59-7.47 (m, 2H), 7.34 (dd, J = 8.4, 2.0, 1H), 7.17 (d, J = 8.5, 1H), 4.25 (dt, J = 16.3, 4.8, 1H), 4.17-3.95 (m, 2H), 3.90-3.78 (m) and 3.70 (dd, J = 14.2, 4.8) together 2 H, 3.63-3.38 (m, 2H), 2.28 (q, J = 14.8, 1H), 2.20-2.05 (m, 1H), 0.94 and 0.94 (2 x s, 9H)

| | | |
|---|---|---|
| “A101” | (R)-7-(2,3-Difluoro-4-methylphenyl)-2-((S)-1- piperidin-4-ylpyrrolidine-2-carbonyl)-1,3,4,11a- tetrahydro-2H,10H-2,4a,10-triaza- dibenzo[a,d]cycloheptene-5,11-dione | D; 2.28 |
| | 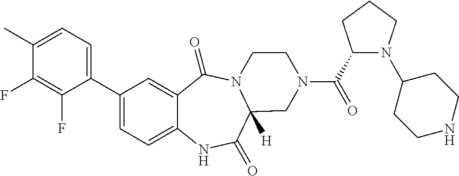 | |
| “A102” | (R)-7-(4-Chloro-3-fluorophenyl)-2-((S)-1-piperidin-4- ylpyrrolidine-2-carbonyl)-1,3,4,11a-tetrahydro- 2H,10H-2,4a,10-triazadibenzo[a,d]cycloheptene- 5,11-dione | D; 2.41 |
| | 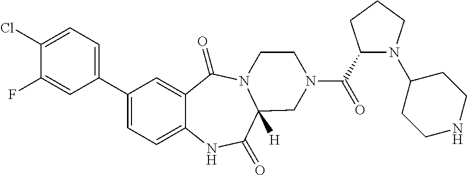 | |
| “A103” | 7-(2-Chlorophenyl)-2-(3,3-dimethylbutyryl)- 1,3,4,11a-tetrahydro-2H,10H-2,4a,10-triaza- dibenzo[a,d]cycloheptene-5,11-dione | D; 3.2 |
| | 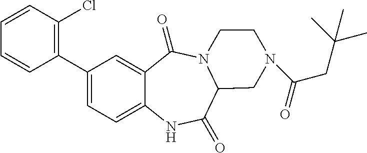 | |
| “A104” | 7-(3,4-Dichlorophenyl)-2-(3,3-dimethylbutyryl)- 1,3,4,11a-tetrahydro-2H,10H-2,4a,10-triaza- dibenzo[a,d]cycloheptene-5,11-dione | D; 3.43 |
| | 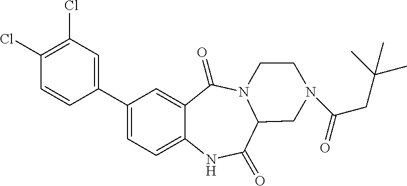 | |
| “A105” | 2-(3,3-Dimethylbutyryl)-7-p-tolyl-1,3,4,11a- tetrahydro-2H,10H-2,4a,10-triaza- dibenzo[a,d]cycloheptene-5,11-dione | I; 1.74 |
| | 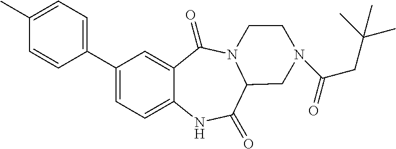 | |
| “A106” | 7-(4-Chlorophenyl)-2-(1-phenylcyclopentane- carbonyl)-1,3,4,11a-tetrahydro-2H,10H-2,4a,10- triazadibenzo[a,d]cycloheptene-5,11-dione | D; 359 |
| | 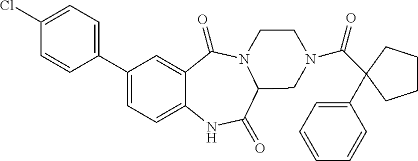 | |
| “A107” | 7-(4-Chlorophenyl)-2-(1,4-dimethylpiperidine-4- carbonyl)-1,3,4,11a-tetrahydro-2H,10H-2,4a,10- triazadibenzo[a,d]cycloheptene-5,11-dione | I; 1.35 |
| | 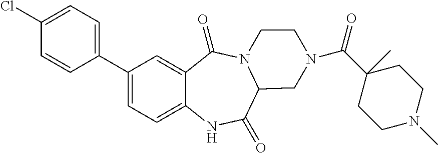 | |
| “A108” | 2-(3,3-Dimethylbutyryl)-7-(1-methyl-1H-pyrazol-4-yl)- 1,3,4,11a-tetrahydro-2H,10H-2,4a,10-triaza- dibenzo[a,d]cycloheptene-5,11-dione | D; 2.79 |
| | 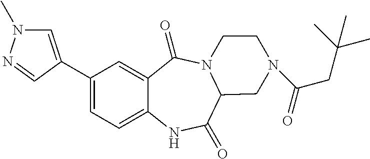 | |
| “A109” | 7-(4-Chlorophenyl)-2-(2,2-dimethylpropionyl)- 1,3,4,11a-tetrahydro-2H,10H-2,4a,10-triaza- dibenzo[a,d]cycloheptene-5,11-dione | D; 314 |
| | 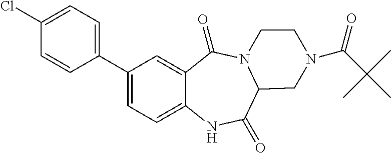 | |
| “A110” | 7-(4-Chlorophenyl)-2-[1-(pyridine-4-carbonyl)- pyrrolidine-2-carbonyl]-1,3,4,11a-tetrahydro-2H,10H- 2,4a,10-triazadibenzo[a,d]cycloheptene-5,11-dione | H; 3.0 |
| | 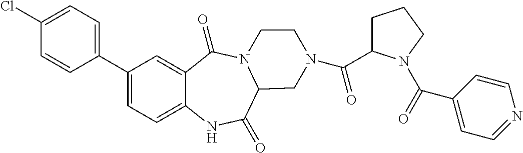 | |
| “A111” | N-(4-Chlorophenyl)-2-[7-(4-chlorophenyl)-5,11- dioxo-3,4,5,10,11,11a-hexahydro-1H-2,4a,10-triaza- dibenzo[a,d]cycloheptene-2-carbonyl]pyrrolidine-1- carboxamide | D; 3.33 + 3.39 |
| | 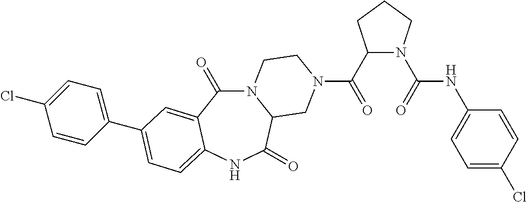 | |
| “A112” | 7-(4-Chlorophenyl)-2-((R)-2-cyclohexyl-2-hydroxy- acetyl)-1,3,4,11a-tetrahydro-2H,10H-2,4a,10- triazadibenzo[a,d]cycloheptene-5,11-dione | D; 3.14 |
| | 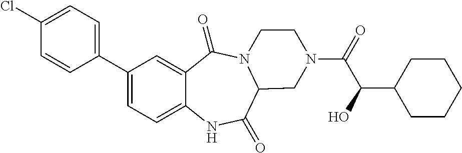 | |
| “A113” | 7-(4-Chlorophenyl)-2-((S)-2-cyclohexyl-2-hydroxy- acetyl)-1,3,4,11a-tetrahydro-2H,10H-2,4a,10- triazadibenzo[a,d]cycloheptene-5,11-dione | D; 3.29 |
| | 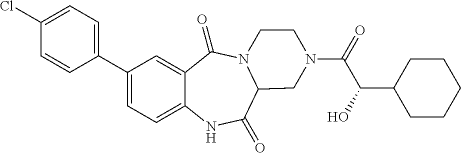 | |
| “A114” | 7-(4-Chlorophenyl)-2-((S)-pyrrolidine-2-carbonyl)- 1,3,4,11a-tetrahydro-2H,10H-2,4a,10-triaza- dibenzo[a,d]cycloheptene-5,11-dione | D; 2.45 |
| | 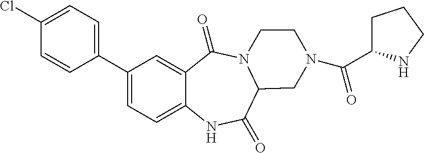 | |
| “A115” | 7-(4-Chloro-2-fluorophenyl)-2-[(S)-1-(2-dimethyl- aminoethyl)pyrrolidine-2-carbonyl]-1,3,4,11a- tetrahydro-2H,10H-2,4a,10-triaza- dibenzo[a,d]cycloheptene-5,11-dione | D; 254 |
| | 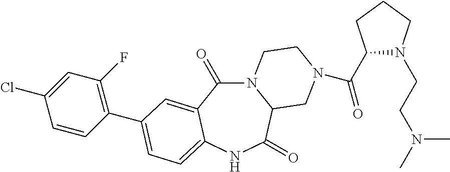 | |
| “A116” | (R)-2-Acetyl-7-(4-chloro-2-fluorophenyl)-1,3,4,11a- tetrahydro-2H,10H-2,4a,10-triaza- dibenzo[a,d]cycloheptene-5,11-dione | D; 291 |
| | 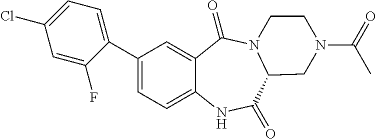 | |
| “A117” | (R)-7-(4-Chloro-2-fluorophenyl)-2-((S)-1-piperidin-4- ylpyrrolidine-2-carbonyl)-1,3,4,11a-tetrahydro- 2H,10H-2,4a,10-triazadibenzo[a,d]cycloheptene- 5,11-dione | D; 2.29 |
| | 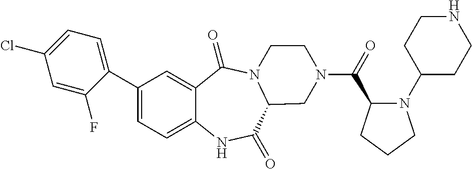 | |
| “A118” | (R)-7-(4-Chloro-2-fluorophenyl)-2-[(S)-1-(1- methylpiperidin-4-yl)pyrrolidine-2-carbonyl]- 1,3,4,11a-tetrahydro-2H,10H-2,4a,10-triaza- dibenzo[a,d]cycloheptene-5,11-dione | D; 2.28 |
| | 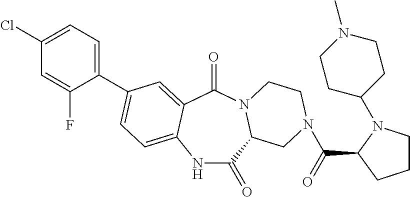 | |
| “A119” | (R)-2-[(S)-1-(2-Aminoethyl)pyrrolidine-2-carbonyl]-7- (4-chloro-2-fluorophenyl)-1,3,4,11a-tetrahydro- 2H,10H-2,4a,10-triazadibenzo[a,d]cycloheptene- 5,11-dione | D; 2.29 |
| | 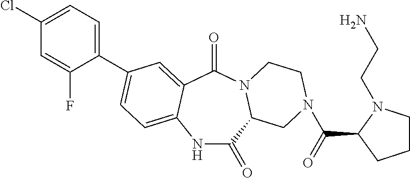 | |

¹H NMR (500 MHz, DMSO-d₆) δ [ppm] 10.73 (br. s, 1H), 8.29 (s, 2H), 7.94 (s, 1H), 7.71 (dd, J = 33.4, 8.0, 1H), 7.66-7.46 (m, 2H), 7.41 (d, J = 8.3, 1H), 7.26 (dd, J = 8.3, 4.5, 1H), 4.48-4.18 (m, 2H), 4.05-3.45 (m, 7H), 3.23-3.01 (m, 1H), 2.96-2.59 (m, 4H), 2.23 (d, J = 7.4, 1H), 1.73 (m, 3H)

| | | |
|---|---|---|
| “A120” | (R)-7-(4-Chloro-2-fluorophenyl)-2-[(S)-1-(tetrahydro- pyran-4-yl)pyrrolidine-2-carbonyl]-1,3,4,11a-tetra- hydro-2H,10H-2,4a,10-triazadibenzo[a,d]- cycloheptene-5,11-dione | D; 251 |
| | 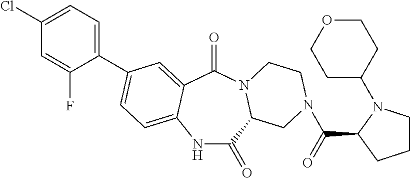 | |
| “A121” | (R)-7-(4-Chloro-2-fluorophenyl)-2-((S)-1-cyclohexyl- pyrrolidine-2-carbonyl)-1,3,4,11a-tetrahydro-2H,10H- 2,4a,10-triazadibenzo[a,d]cycloheptene-5,11-dione | D; 2.64 |
| | 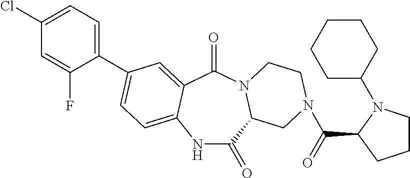 | |
| “A122” | (R)-2-((S)-2-Amino-3-hydroxypropionyl)-7-(4- chloro-2-fluorophenyl)-1,3,4,11a-tetrahydro- 2H,10H-2,4a,10-triazadibenzo[a,d]cycloheptene- 5,11-dione | D; 2.45 |
| | 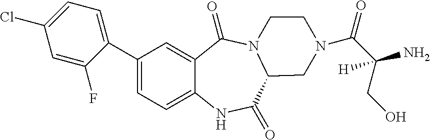 | |
| “A123” | (R)-7-(4-Chloro-2-fluorophenyl)-2-((S)-1-piperidin-3- ylpyrrolidine-2-carbonyl)-1,3,4,11a-tetrahydro- 2H,10H-2,4a,10-triazadibenzo[a,d]cycloheptene- 5,11-dione | D; 2.27 |
| | 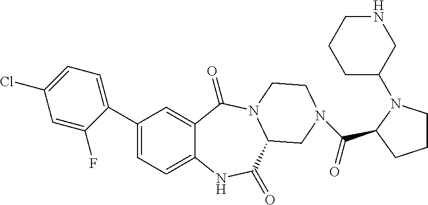 | |
| “A124” | (R)-2-((S)-2-Amino-3-methoxypropionyl)-7-(4-chloro- 2-fluorophenyl)-1,3,4,11a-tetrahydro-2H,10H- 2,4a,10-triazadibenzo[a,d]cycloheptene-5,11-dione | D; 2.45 |
| | 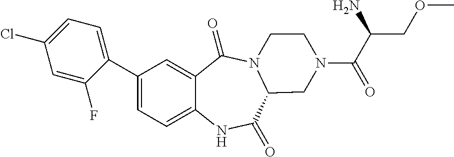 | |
| “A125” | (S)-7-(4-Chloro-2-fluorophenyl)-2-(3,3-dimethyl- butyryl)-1,3,4,11a-tetrahydro-2H,10H-2,4a,10- triazadibenzo[a,d]cycloheptene-5,11-dione | D; 3.34 |
| | 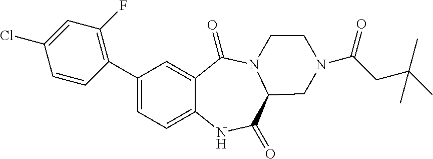 | |
| “A126” | (S)-7-(4-Chloro-2-fluorophenyl)-2-((S)-1-piperidin-4- ylpyrrolidine-2-carbonyl)-1,3,4,11a-tetrahydro- 2H,10H-2,4a,10-triazadibenzo[a,d]cycloheptene- 5,11-dione | D; 2.23 |
| | 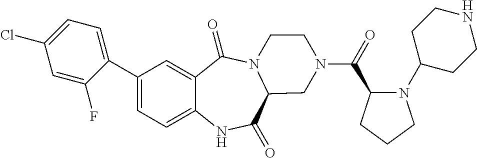 | |
| “A128” | 7-(6-Chloropyridin-3-yl)-2-(3,3-dimethylbutyryl)- 1,3,4,11a-tetrahydro-2H,10H-2,4a,10-triaza- dibenzo[a,d]cycloheptene-5,11-dione | I; 1.51 |
| | 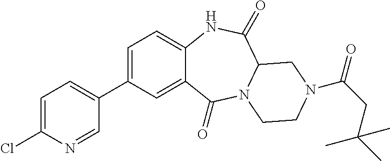 | |
| “A129” | 7-(5-Chlorothiophen-2-yl)-2-(3,3-dimethylbutyryl)- 1,3,4,11a-tetrahydro-2H,10H-2,4a,10-triaza- dibenzo[a,d]cycloheptene-5,11-dione | I; 1.68 |
| | 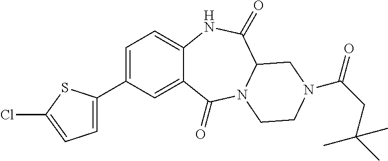 | |
| “A130” | 2-(3,3-Dimethylbutyryl)-7-(4-fluorophenyl)-1,3,4,11a- tetrahydro-2H,10H-2,4a,10-triaza- dibenzo[a,d]cycloheptene-5,11-dione | I; 1.68 |
| | 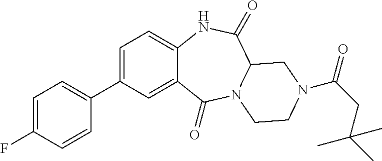 | |
| “A131” | 2-(3,3-Dimethylbutyryl)-7-phenyl-1,3,4,11a- tetrahydro-2H,10H-2,4a,10-triaza- dibenzo[a,d]cycloheptene-5,11-dione | I; 1.67 |
| | 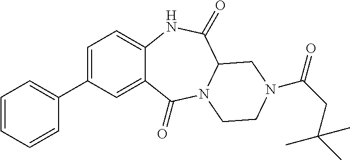 | |
| “A132” | 4-[2-(3,3-Dimethylbutyryl)-5,11-dioxo- 1,2,3,4,5,10,11,11a-octahydro-2,4a,10-triaza- dibenzo[a,d]cyclohepten-7-yl]benzonitrile | I; 1.55 |
| | 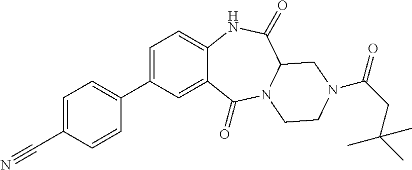 | |
| “A133” | 2-(3,3-Dimethylbutyryl)-7-trifluoromethoxy-1,3,4,11a- tetrahydro-2H,10H-2,4a,10-triaza- dibenzo[a,d]cycloheptene-5,11-dione | I; 1.65 |
| | 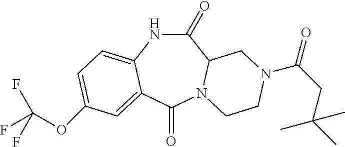 | |
| “A134” | 7-(4-Chlorophenyl)-10-(2-dimethylaminoethyl)-2- (3,3-dimethylbutyryl)-1,3,4,11a-tetrahydro-2H,10H- 2,4a,10-triazadibenzo[a,d]cycloheptene-5,11-dione | I; 1.43 |
| | 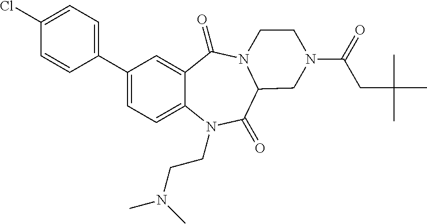 | |
| “A135” | 7-(4-Chlorophenyl)-2-(3,3-dimethylbutyryl)-10-(2- methoxyethyl)-1,3,4,11a-tetrahydro-2H,10H-2,4a,10- triazadibenzo[a,d]cycloheptene-5,11-dione | I; 1.81 |
| | 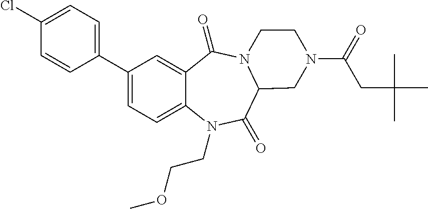 | |
| “A136” | 7-(4-Chlorophenyl)-2-(2,2,2-trifluoroacetyl)- 1,3,4,11a-tetrahydro-2H,10H-2,4a,10-triazadibenzo- [a,d]cycloheptene-5,11-dione | I; 1.65 |
| | 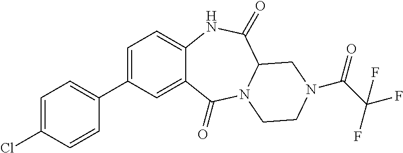 | |
| “A137” | 2-((S)-2-Amino-3,3-dimethylbutyryl)-7-(4-chloro- phenyl)-1,3,4,11a-tetrahydro-2H,10H-2,4a,10- triazadibenzo[a,d]cycloheptene-5,11-dione | I; 1.46 |
| | 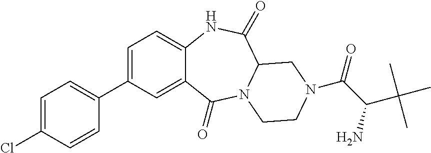 | |
| “A138” | 7-(4-Chlorophenyl)-2-((S)-2-methoxy-3,3-dimethyl- butyryl)-1,3,4,11a-tetrahydro-2H,10H-2,4a,10- triazadibenzo[a,d]cycloheptene-5,11-dione | I; 1.65 |
| | 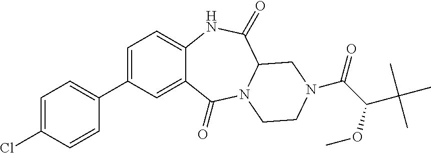 | |
| “A139” | 3-(4-Chlorophenyl)-8-(3,3-dimethylbutyryl)-7,8,9,9a- tetrahydro-6H,11H-1,5a,8,11-tetraazadibenzo- [a,d]cycloheptene-5,10-dione | I; 1.63 |
| | 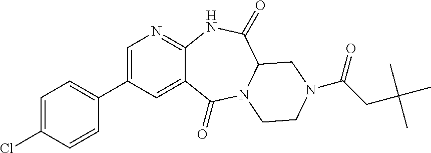 | |
| “A140” | 2-(3,3-Dimethylbutyryl)-7-(4-ethynylphenyl)- 1,3,4,11a-tetrahydro-2H,10H-2,4a,10-triaza- dibenzo[a,d]cycloheptene-5,11-dione | I; 1.71 |
| | 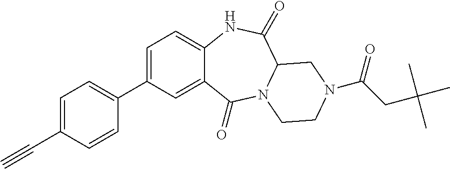 | |
| “A141” | 7-(4-Chlorophenyl)-2-(5,5-dimethylthiazolidine-4- carbonyl)-1,3,4,11a-tetrahydro-2H,10H-2,4a,10- triazadibenzo[a,d]cycloheptene-5,11-dione | I; 1.45 |
| | 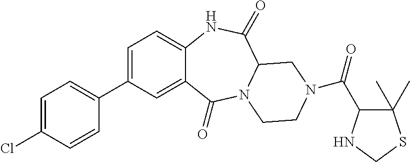 | |

### EXAMPLE 15

The following is obtained analogously to Example 2

| | | HPLC method; |
|---|---|---|
| Compound No. | Name and/or structure | RT [min] |
| “A142” | 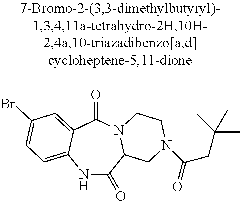 | D; 2.79 |

### EXAMPLE 16

The following are obtained analogously to Example 5

| | | HPLC method; |
|---|---|---|
| Compound No. | Name and/or structure | RT [min] |
| “A143” | 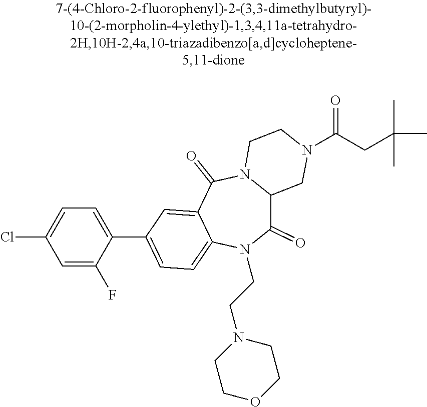 | D; 2.71 |
| “A144” | 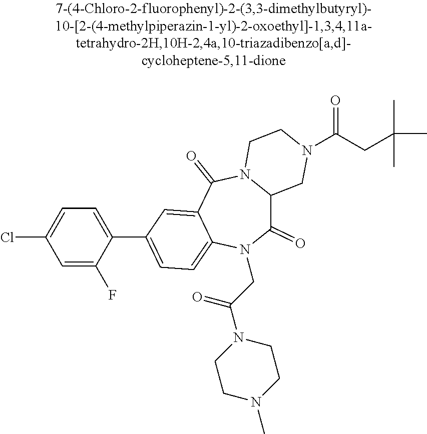 | D; 2.69 |
| “A145” | 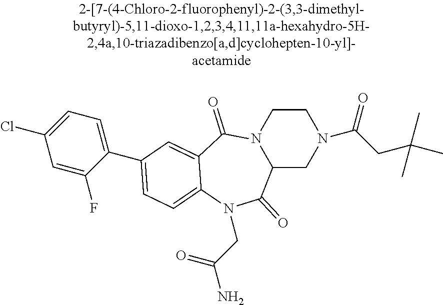 | D; 312 |
| “A146” | 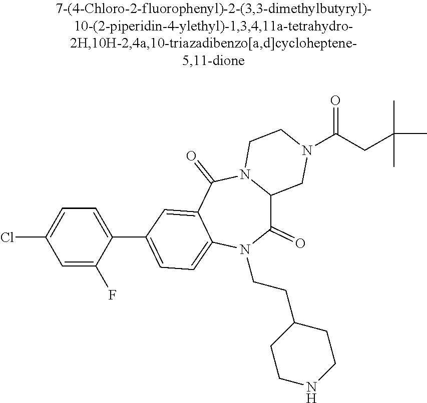 | D; 2.73 |
| “A147” | 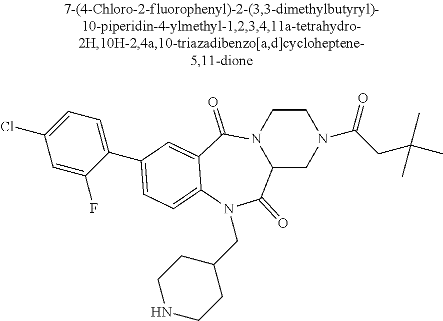 | D; 2.71 |
| “A148” | 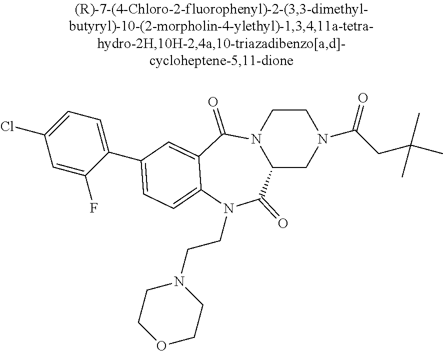 | D; 2.74 |

¹H NMR (500 MHz, DMSO-d₆) δ [ppm] 7.85 (s, 1 H), 7.78 (d, J = 8.6, 1 H),
7.70-7.61 (m, 2 H), 7.58 (dd, J = 10.8, 2.0, 1 H), 7.41 (d, J = 8.3, 1 H), 4.50-
4.41 (m, 1 H), 4.31 (m, 1 H), 4.26-4.10 (m) and 3.99 (dd, J = 8.8, 6.2)
together 2 H, 3.87-3.49 (m, 4 H), 3.43-3.15 (m, 5 H), 2.45-2.00 (m, 8 H),
1.01 and 0.99 (2 × s, 9 H)

| | | |
|---|---|---|
| “A149” | 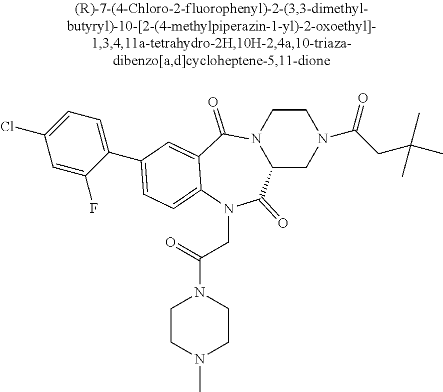 | D; 2.71 |
| “A150” | 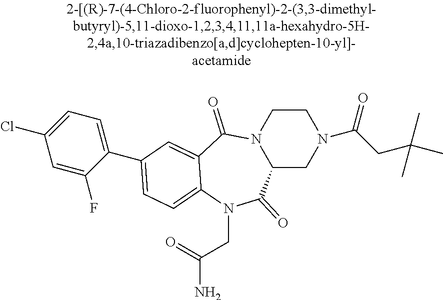 | D; 3.11 |
| “A151” | 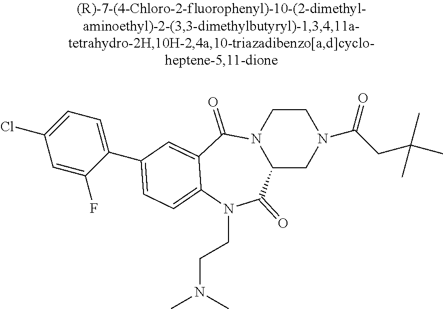 | D; 2.73 |
| “A152” | 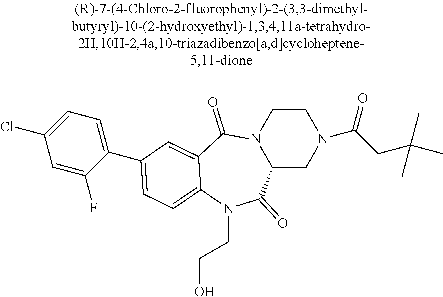 | D; 3.2 |

¹H NMR (500 MHz, DMSO-d₆) δ [ppm] 7.85 (s, 1 H), 7.83-7.72 (m, 2 H),
7.64 (q, J = 8.6, 1 H), 7.58 (dd, J = 10.7, 2.0, 1 H), 7.41 (d, J = 8.3, 1 H), 4.75
(s, 1 H), 4.29 (m, 1 H), 4.24-3.92 (m, 3 H), 3.90-3.75 (m, 2 H), 3.72-3.44
(m, 4 H), 2.40 (d, J = 14.8) and 2.31 (d, J = 14.8) together 1 H, 2.20 (q, J = 14.5,
1 H), 1.01 and 1.00 (2 × s, 9 H)

| | | |
|---|---|---|
| “A153” | 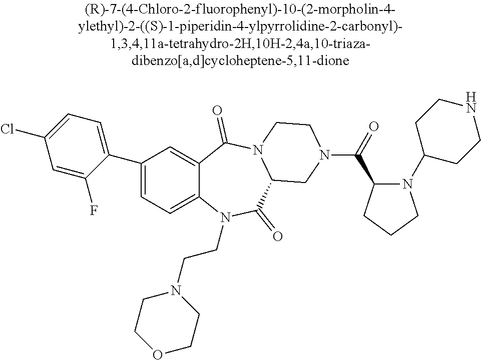 | D; 2.14 |
| “A154” | 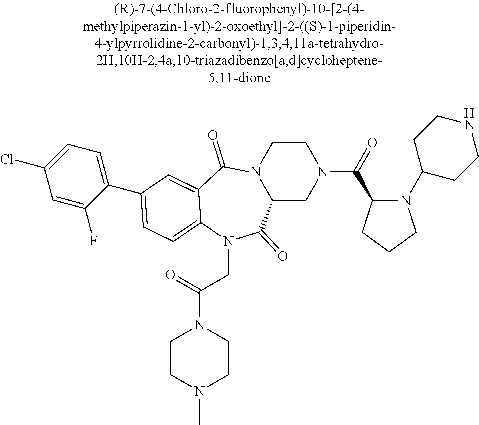 | D; 2.13 |
| “A155” | 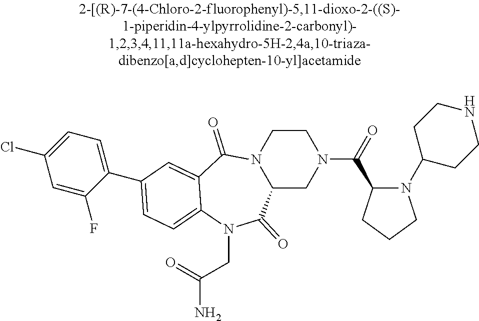 | D; 2.29 |
| “A156” | 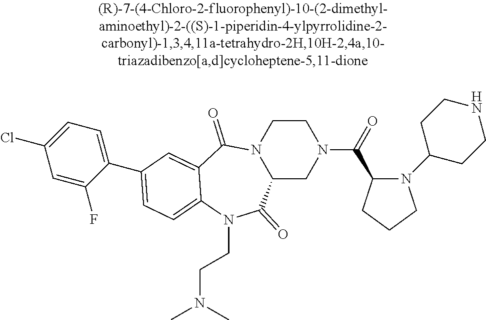 | D; 2.08 |
| “A157” | 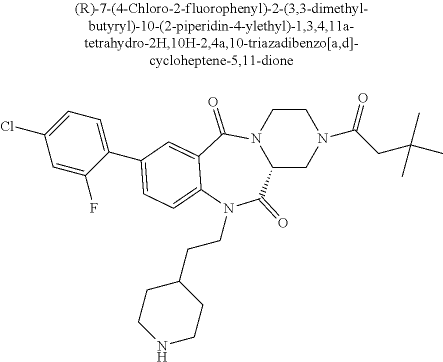 | D; 2.75 |

¹H NMR (500 MHz, DMSO-d₆) δ [ppm] 7.86 (s, 1 H), 7.81 (d, J = 8.6, 1 H),
7.70-7.61 (m, 2 H), 7.58 (dd, J = 10.8, 2.0, 1 H), 7.42 (d, J = 8.3, 1 H), 4.38-
4.10 (m, 3 H), 4.03-3.91 (m, 1 H), 3.85-3.50 (m, 6 H), 3.34-3.19 (m,
2 H), 3.11 (m, 2 H), 2.65-2.52 (m, 2 H), 2.44-2.13 (m, 2 H), 1.70 (d, J = 11.2,
1 H), 1.51 (d, J = 12.9, 1 H), 1.35 (m, 2 H), 1.01 and 1.00 (2 × s, 9 H)

| | | |
|---|---|---|
| “A158” | 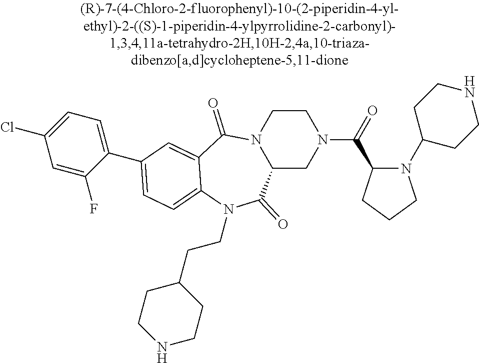 | D; 2.13 |
| “A159” | 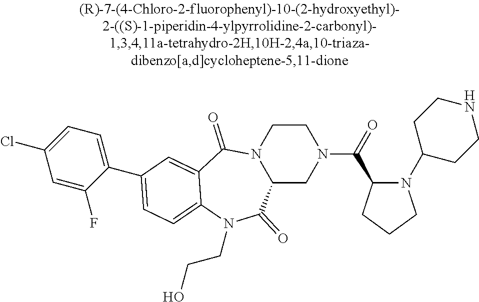 | D; 2.27 |
| “A160” | 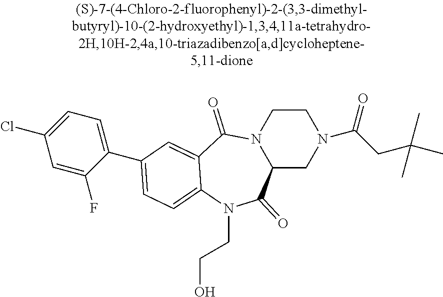 | D; 3.24 |
| “A161” | 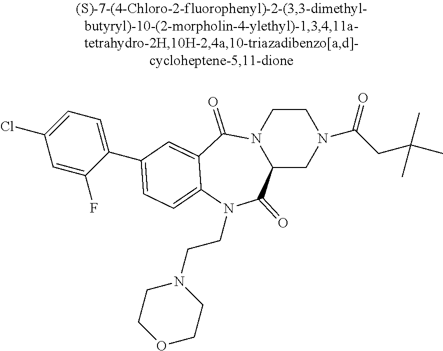 | D; 2.69 |
| “A162” | 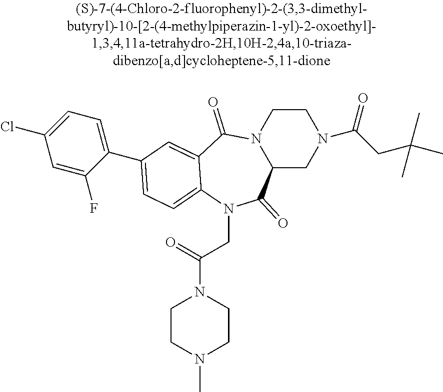 | D; 267 |
| “A163” | 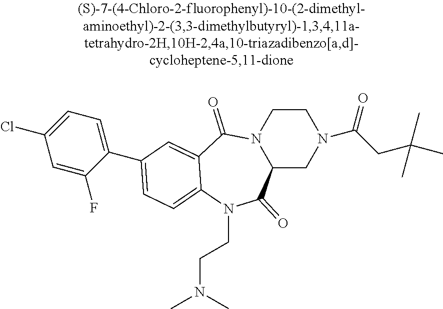 | D; 2.66 |
| “A164” | 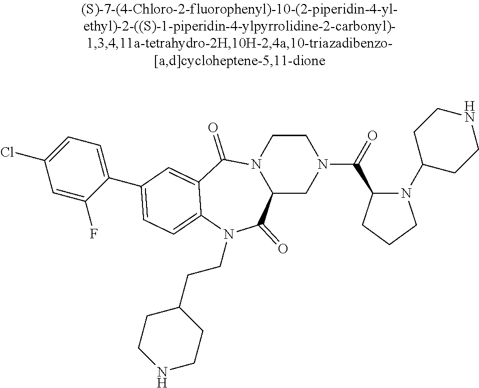 | D; 2.13 |
| “A165” | 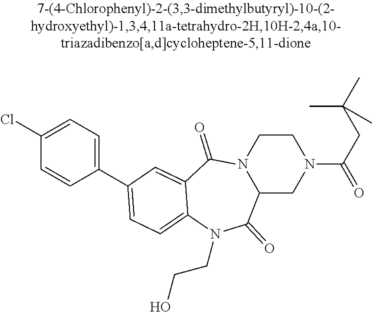 | I; 1.66 |
| “A166” | 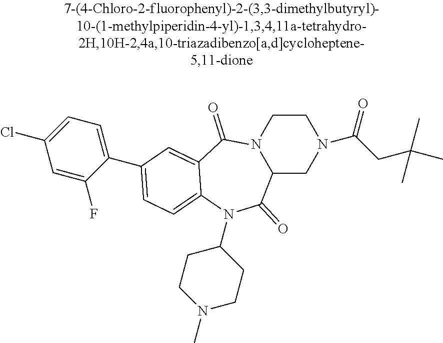 | D; 2.63 |

### EXAMPLE 17

The following are obtained analogously to Example 9

| | | HPLC method; |
|---|---|---|
| Compound No. | Name and/or structure | RT [min] |
| “A167” | 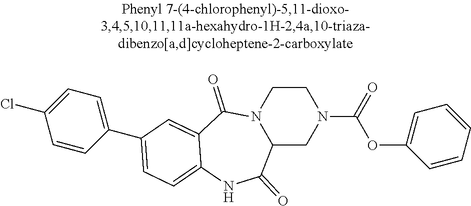 | D; 3.39 |

¹H NMR (500 MHz, DMSO-d₆) δ [ppm] 10.67 and 10.65 (2 × s, 1 H), 8.04 (s,
1 H), 7.88 (dd, J = 8.4, 2.3, 1 H), 7.73 (d, J = 8.5, 2 H), 7.54 (d, J = 8.5, 2 H),
7.40 (t, J = 7.9, 2 H), 7.20 (m, 4 H), 4.45-4.32 (m) and 4.14 (dd, J = 13.9,
4.3) together 2 H, 4.06-3.94 (m, 1 H), 3.92-3.78 (m, 2 H), 3.74 (dd, J = 13.7,
4.7) and 3.69-3.47 (m) together 2 H; rotamer mixture

| | | |
|---|---|---|
| “A168” | 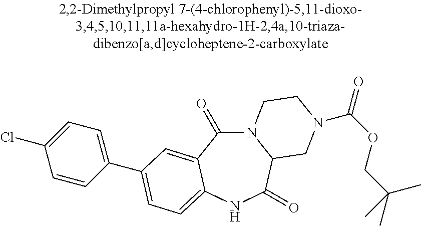 | D; 3.56 |

¹H NMR (500 MHz, DMSO-d₆) δ [ppm] 10.60 (s, 1 H), 8.02 (d, J = 2.2, 1 H),
7.86 (dd, J = 8.4, 2.3, 1 H), 7.72 (d, J = 8.5, 2 H), 7.53 (d, J = 8.5, 2 H), 7.22
(d, J = 8.0, 1 H), 4.32 (s, 1 H), 4.13 (dd, J = 51.4, 10.9, 1 H), 3.94 (ddd, J = 12.4,
7.8, 4.3, 1 H), 3.88-3.56 (m, 4 H), 3.46 (s, 1 H), 2.55 (s, 1 H), 0.93 (s, 9 H)

| | | |
|---|---|---|
| “A170” | 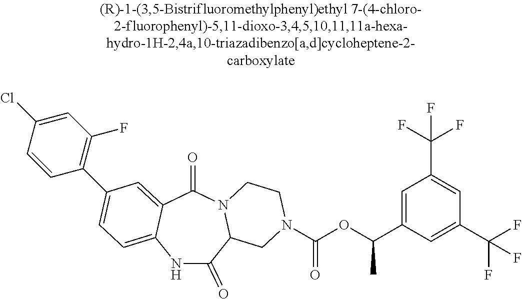 | D; 3.96 |

¹H NMR (500 MHz, DMSO-d₆) δ [ppm] 10.68, 10.64 nd 10.60 (3 × s, 1 H),
8.19-7.87 (m, 4 H), 7.77-7.69 (m, 1 H), 7.65-7.21 (m, 4 H), 5.97-5.90
(m, 1 H), 4.41-4.00 (m, 2 H), 3.99-3.39 (m, 5 H), 1.63-1.49 (m, 3 H).

### EXAMPLE 18

The following are obtained analogously to Example 12

| | | HPLC method; |
|---|---|---|
| Compound No. | Name and/or structure | RT [min] |
| “A173” | 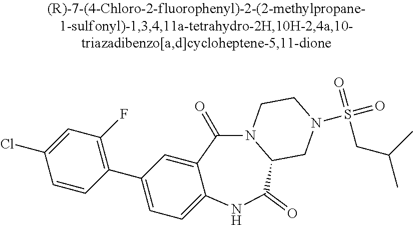 | D; 3.41 |
| “A174” | 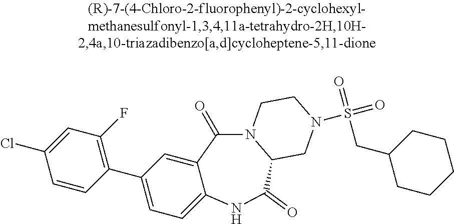 | D; 3.66 |

### EXAMPLE 19

The following is obtained analogously to Example 13

| | | HPLC method; |
|---|---|---|
| Compound No. | Name and/or structure | RT [min] |
| “A176” | 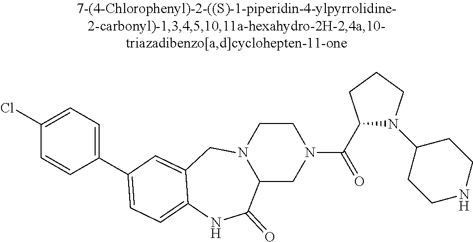 | D; 2.28 |

### Pharmacological Data

### Autotaxin Inhibition (Enzyme Test)

TABLE 1

| Compound | | % Control at | Compound | | % Control at |
|---|---|---|---|---|---|
| No. | IC50 | 10 μM | No. | IC50 | 10 μM |
| “B1” | | 75 | “B22” | | 71 |
| “B2” | | 84 | “B23” | | 76 |
| “A1” | C | | “B24” | | 83 |
| “B3” | | 47 | “A10” | C | 34 |
| “B4” | | 71 | “B25” | C | 47 |
| “B5” | | 64 | “A11” | | 69 |
| “B6” | | 85 | “A12” | C | |
| “B7” | | 70 | “A13” | C | |
| “B8” | | 70 | “A14” | C | 38 |
| “B9” | | 74 | “A15” | C | 55 |
| “A3” | | 86 | “25” | C | 53 |
| “B10” | | 76 | “26” | C | 73 |
| “A5” | C | | “A16” | C | 18 |
| “A4” | | 75 | “A17” | B | |
| “B11” | | 85 | “B26” | B | |
| “B12” | | 84 | “A18” | C | 70 |
| “A6” | | 73 | “A19” | C | 65 |
| “A7” | C | | “A20” | C | 63 |
| “B13” | | 53 | “A21” | C | 82 |
| “B14” | | 80 | “A22” | C | 61 |
| “B15” | | 79 | “A23” | C | 54 |
| “A8” | | 67 | “A24” | C | 67 |
| “B16” | | 82 | “A25” | C | 11 |
| “B17” | C | 26 | “A26” | C | 12 |
| “B18” | | 56 | “A27” | C | 12 |
| “B19” | C | 41 | “A28” | C | 4 |
| “B20” | | 71 | “A29” | C | 51 |
| “B21” | | 57 | “A30” | C | 23 |
| “A9” | | 84 | “A31” | C | 17 |
| “A32” | | 76 | “A45” | C | 61 |
| “A33” | C | 72 | “A46” | C | |
| “10” | B | 1 | “A47” | C | |
| “A34” | B | | “A48” | C | 20 |
| “A35” | C | 11 | “A49” | C | |
| “A36” | B | | “A50” | | 80 |
| “A37” | C | 66 | “A51” | C | 37 |
| “B27” | C | 59 | “A52” | C | |
| “A38” | C | 35 | “A53” | C | |
| “A39” | C | 22 | “A54” | | 84 |
| “A40” | C | 48 | “A55” | | 71 |
| “A41” | C | 68 | “A56” | B | |
| “A42” | C | 12 | “A57” | | |
| “A43” | C | 31 | “17” | | |
| “A44” | C | 48 | | | |
| “40” | C | 22 | “A70” | C | 28 |
| “41” | C | 48 | “A71” | C | 7 |
| “47” | B | 8 | “A72” | C | 70 |
| “48” | B | 2 | | | |
| “57” | C | 40 | “A73” | | 78 |
| “A58” | C | −27 | “A74” | C | −2 |
| “A59” | C | −9 | “A75” | | 55 |
| “A60” | B | −27 | “A76” | C | 41 |
| “A61” | C | −11 | “A77” | C | 11 |
| “A62” | C | 27 | “A78” | C | 16 |
| “A63” | C | 67 | “A79” | B | −14 |
| “A64” | C | 31 | “A80” | | 44 |
| “A65” | C | 48.5 | “A81” | | 33 |
| “A66” | B | 9.5 | “A82” | B | −16 |
| “A67” | C | 19 | “A83” | | 81 |
| “A68” | B | −18 | “A84” | | 29 |
| “A69” | | 58 | “A85” | C | 13 |
| “A86” | | 78 | “A102” | B | 7 |
| “A87” | C | 74 | “A103” | C | −10 |
| “A88” | C | −6 | “A104” | B | −13 |
| “A89” | C | 4 | “A105” | C | 22 |
| “A90” | C | 24 | “A106” | B | −37 |
| “A91” | C | −6 | “A107” | C | 51 |
| “A92” | C | 1 | “A108” | | 90 |
| “A93” | | 17 | “A109” | C | −21 |
| “A94” | C | −18 | “A110” | C | 51 |
| “A95” | C | 15 | “A111” | C | 3 |
| “A96” | B | 1 | “A112” | C | 5 |
| “A97” | B | 19 | “A113” | C | −6 |
| “A98” | C | 90 | “A114” | C | 28 |
| “A99” | C | 19 | “A115” | C | 80 |
| “A100” | C | 11 | “A116” | C | 71 |
| “A101” | B | 8 | “A117” | C | 16 |
| “A118” | B | 8 | “A134” | C | −19 |
| “A119” | C | 45 | “A135” | C | −9 |
| “A120” | B | 8 | “A136” | | 63 |
| “A121” | C | 32 | “A137” | C | −18 |
| “A122” | | 69 | “A138” | B | −21 |
| “A123” | B | 6 | “A139” | C | 45 |
| “A124” | C | 39 | “A140” | C | −10 |
| “A125” | C | 25 | “A141” | B | −21 |
| “A126” | C | 20 | “A142” | | 85 |
| | | | “A143” | B | 3 |
| “A128” | C | 42 | “A144” | C | 11 |
| “A129” | C | 6 | “A145” | B | 2 |
| “A130” | C | −1 | “A146” | C | 10 |
| “A131” | C | 14 | “A147” | C | 62 |
| “A132” | | 84 | “A148” | C | 10 |
| “A133” | C | 27 | “A149” | C | 15 |
| “A150” | B | 3 | “A166” | C | 10 |
| “A151” | C | 16 | “A167” | C | 26 |
| “A152” | B | 3 | “A168” | C | 52 |
| “A153” | C | 21 | “A171” | C | 48 |
| “A154” | C | 27 | “A173” | B | 1 |
| “A155” | B | 10 | “A174” | B | 21 |
| “A156” | B | 7 | “A176” | C | 63 |
| “A157” | B | 2 | | | |
| “A158” | C | 21 | | | |
| “A159” | C | 16 | | | |
| “A160” | B | 2 | | | |
| “A161” | B | 3 | | | |
| “A162” | C | 30 | | | |
| “A163” | C | 10 | | | |
| “A164” | C | 12 | | | |
| “A165” | B | −20 | | | |

IC50: <100 nM = A 100 nM-1 μM = B >1 μM = C

### EXAMPLE A

### Autotaxin Test (Enzyme Test)

### Test Description

The autotaxin activity is measured indirectly using Amplex Red reagent. Amplex Red is measured here as fluorogenic indicator for the H₂O₂ formed. In detail, autotaxin converts the substrate lysophosphatidylcholine (LPC) into phosphocholine and lysophosphatidylic acid (LPA). After this reaction, the phosphocholine is reacted with alkaline phosphatase to give inorganic phosphate and choline. In the next step, choline is oxidised by choline oxidase to give betaine, with formation of H₂O₂. H₂O₂ reacts with Amplex Red reagent in the presence of peroxidase (horseradish peroxidase) in a 1:1 stoichiometry and forms the highly fluorescent resorufin. The fluorescence is measured in a reaction-dependent kinetic mode in order that fluorescent signals from possible other fluorescent substances which are not involved in the reaction can be corrected out.

### Test Procedure

3 μl of a standard solution or of the test substances (substances with the name A(n)) in individual concentrations dissolved in 20 mM Hepes pH 7.2 with a maximum of 11% of DMSO are pre-incubated together with 20 μl (19 ng) of highly purified recombinant autotaxin in test buffer in a black microtitre plate provided with 384 wells at 22° C. for 30 min. The reaction is then initiated by addition of 10 μl of L-α-lysophosphatidylcholine (LPC), where the final concentration of LPC is 75 μM. The mixture is incubated at 37° C. for 90 min. After the incubation, Amplex Red reagent, peroxidase (horseradish peroxidase) and choline oxidase is added, and the fluorescence is immediately measured at 612 nm with excitation of 485 nm in a “Tecan Ultra multimode” reader. The activity of autotaxin is calculated indirectly via detection of the H₂O₂ formed.

### Material:

Microtitre plate: PS microplate, 384 wells, small volume, black Corning, Cat#3677
Protein: recombinant autotaxin (Baculovirale Hi5 Expression)
Substrate: L-α-lysophosphatidylcholine (chicken egg)); Avanti Polar Lipids #830071 P
Standard: C14 LPA, Avanti Polar Lipids, Cat#857120P
Detection reagent: Amplex Red reagent; Invitrogen #A12222; 5 mg dissolved in 1.923 ml of DMSO peroxidase type VI-A (horseradish) from Sigma #P6782; 5 mg dissolved in 7.45 ml of test buffer, choline oxidase; Sigma # C5896; 50 U dissolved in 2.47 ml of test buffer
Detection reagent mix: 1:50 dilution of detection reagent in test buffer
Test buffer: 10 mM Tris HCl, Merck, Cat #1.08219, pH 8, 1 mM; CaCl₂×2 H2O, Merck #1.02382

The following examples relate to medicaments:

### EXAMPLE B

### Injection Vials

A solution of 100 g of an active ingredient of the formula I and 5 g of disodium hydrogenphosphate in 3 l of bidistilled water is adjusted to pH 6.5 using 2 N hydrochloric acid, sterile filtered, transferred into injection vials, lyophilised under sterile conditions and sealed under sterile conditions. Each injection vial contains 5 mg of active ingredient.

### EXAMPLE C

### Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### EXAMPLE D

### Solution

A solution is prepared from 1 g of an active ingredient of the formula I, 9.38 g of NaH₂PO₄.2 H₂O, 28.48 g of Na₂HPO₄.12 H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE E

### Ointment

500 mg of an active ingredient of the formula I are mixed with 99.5 g of Vaseline under aseptic conditions.

### EXAMPLE F

### Tablets

A mixture of 1 kg of active ingredient of the formula I, 4 kg of lactose, 1.2 kg of potato starch, 0.2 kg of talc and 0.1 kg of magnesium stearate is pressed in a conventional manner to give tablets in such a way that each tablet contains 10 mg of active ingredient.

### EXAMPLE G

### Dragees

Tablets are pressed analogously to Example E and subsequently coated in a conventional manner with a coating of sucrose, potato starch, talc, tragacanth and dye.

### EXAMPLE H

### Capsules

2 kg of active ingredient of the formula I are introduced into hard gelatine capsules in a conventional manner in such a way that each capsule contains 20 mg of the active ingredient.

### EXAMPLE I

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1. Compounds of the formula I 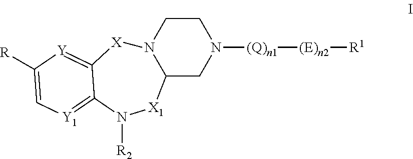
in which
R denotes Hal, Ar or Het¹,
R¹ denotes SO₂A, COOA, COOH, Cyc, Het, Ar, COHet, CONHHet, CONHAr, CHO, CONH₂, CONHA, CONA₂, (CH₂)ₙ₂OH, (CH₂)ₙ₂OA, OAr, NHAr, A, Hal, (CH₂)ₙ₂NH₂, (CH₂)ₙ₂NHA, (CH₂)ₙ₂NA₂ or NHCOA,
R² denotes H, (CH₂)ₙ₃NH₂, (CH₂)ₙ₃NHA, (CH₂)ₙ₃NA₂, (CH₂)ₙ₃OH, (CH₂)ₙ₃OA, (CH₂)ₙ₃Het², CH₂COHet², CH₂CONH₂, CH₂CONHA, CH₂CONA₂ or A,
X, X₁ each, independently of one another, denote CO, CH(OH), CH(OA), CH(NH₂), CH₂ or CF₂,
Y, Y₁ each, independently of one another, denote CH or N,
Q denotes C═O, COO, C═S, C═NH, CH(OH), CH(NH₂), SO, SO₂ or CF₂,
E denotes CO, CH(OH), CA(OH), CH(OA), CA(OA), CH(NH₂), Alk, 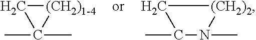
Alk denotes linear or branched alkylene having 1-8 C atoms, in which one or two CH₂ groups may be replaced by O and/or NH,
n1 denotes 0, 1 or 2,
n2 denotes 0, 1, 2, 3 or 4,
n3 denotes 1, 2, 3 or 4,
Ar denotes phenyl, naphthyl or biphenyl, each of which is unsubstituted or mono-, di- or trisubstituted by Hal, A, OH, OA, NH₂, NHA, NA₂, NO₂, CN, COOH, COOA, CONH₂, CONHA, CONA₂, NHCOA, NHSO₂A, SO₂NH₂ and/or SO₂A,
Het denotes a mono-, bi- or tricyclic saturated, unsaturated or aromatic heterocycle having 1 to 4 N, O and/or S atoms, which may be unsubstituted or mono-, di- or trisubstituted by Hal, Het², A, OH, OA, NH₂, NHA, NA₂, NO₂, CN, COOH, COOA, CONH₂, CONHA, CONA₂, NHCOA, NHSO₂A, SO₂NH₂, SO₂A, NHCONH₂, CHO, COA, ═S, ═NH, ═NA and/or ═O (carbonyl oxygen),
Het¹ denotes a mono-, bi- or tricyclic aromatic heterocycle having 1 to 4 N, O and/or S atoms, which may be unsubstituted or mono-, di- or trisubstituted by Hal, A, OH, OA, NH₂, NHA, NA₂, NO₂, CN, COOH, COOA, CONH₂, CONHA, CONA₂, NHCOA, NHSO₂A, SO₂NH₂, SO₂A, NHCONH₂, CHO and/or COA,
Het² denotes pyrrolidinyl, piperidinyl, thiazolidinyl, morpholinyl, oxazolidinyl, tetrahydroquinazolinyl, tetrahydropyranyl, piperazinyl, thiazolyl, furyl, thienyl, pyrrolyl, imidazolyl, pyrazolyl, oxazolyl, isoxazolyl, isothiazolyl, pyridyl, pyrimidinyl, triazolyl, tetrazolyl, oxadiazolyl or thiadiazolyl, each of which is unsubstituted or monosubstituted by A,
Cyc denotes cyclic alkyl having 3-7 C atoms,
A denotes unbranched or branched alkyl having 1-10 C atoms,
in which 1-7H atoms may be replaced by F, Cl and/or Br,
and/or in which one or two CH₂ groups may be replaced by O and/or NH,
or
cyclic alkyl having 3-7 C atoms,
Hal denotes F, Cl, Br or I,
and pharmaceutically usable salts and stereoisomers thereof, including mixtures thereof in all ratios,
where the compounds “B1”-“B27”

| No. | Name and/or structure |
|---|---|
| “B1” | 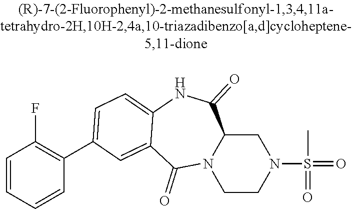 |
| “B2” | 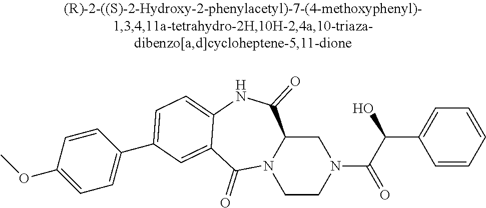 |
| “B3” | 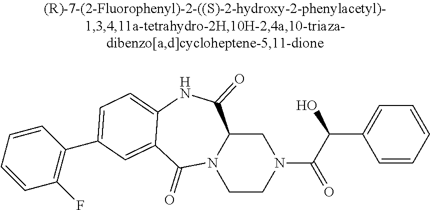 |
| “B4” | 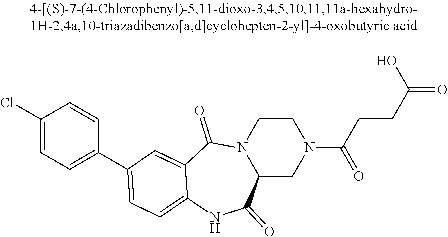 |
| “B5” | 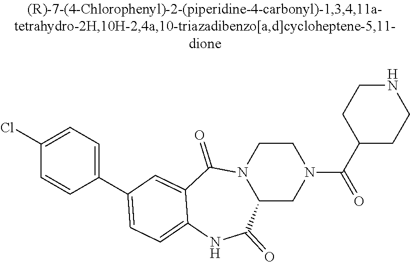 |
| “B6” | 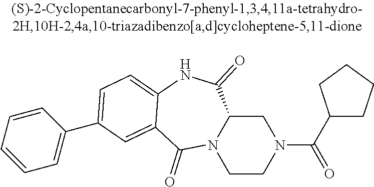 |
| “B7” | 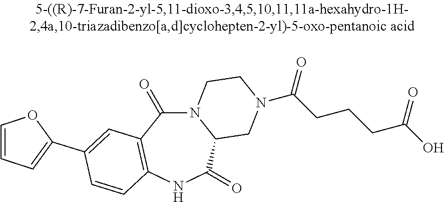 |
| “B8” | 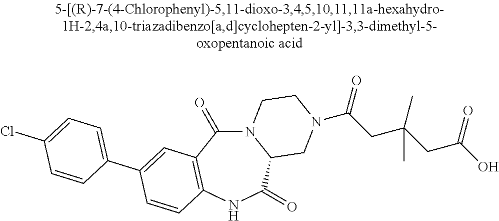 |
| “B9” | 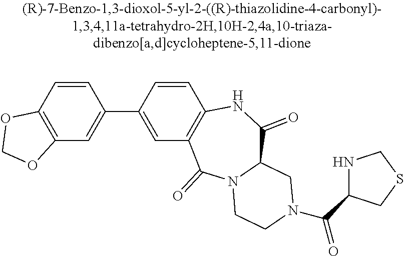 |
| “B10” | 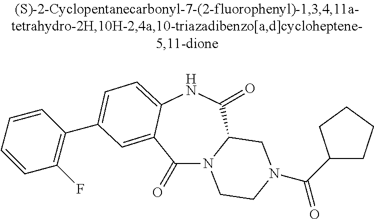 |
| “B11” | 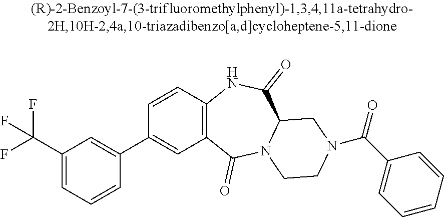 |
| “B12” | 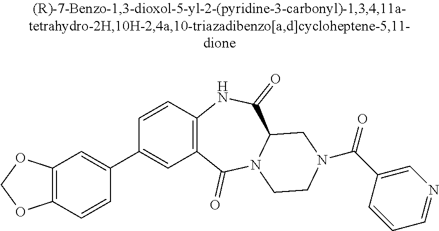 |
| “B13” | 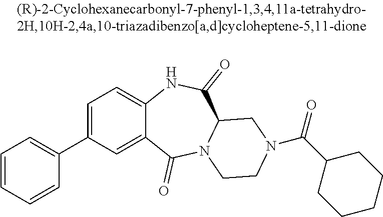 |
| “B14” | 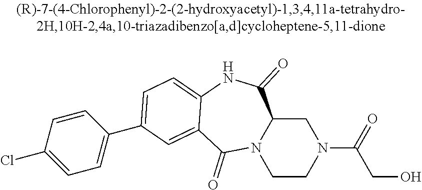 |
| “B15” | 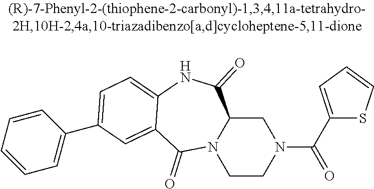 |
| “B16” | 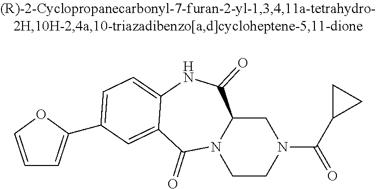 |
| “B17” | 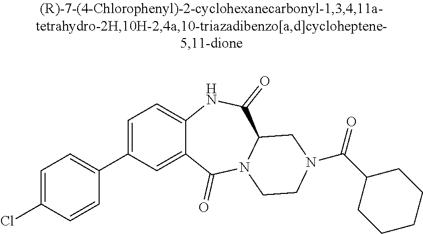 |
| “B18” | 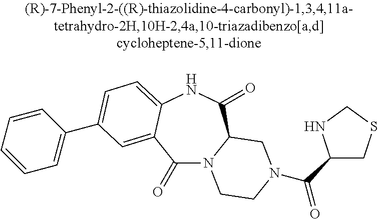 |
| “B19” | 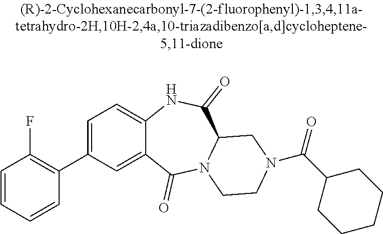 |
| “B20” | 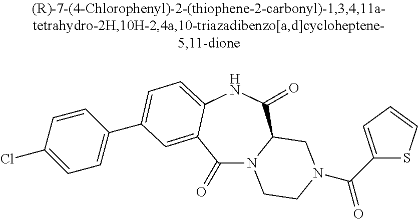 |
| “B21” | 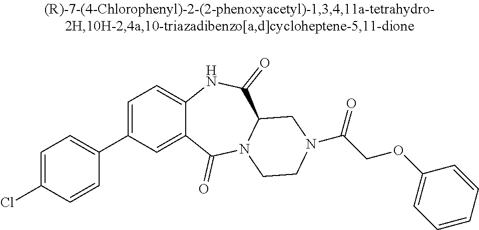 |
| “B22” | 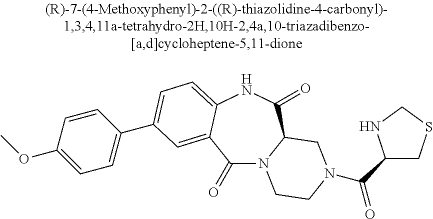 |
| “B23” | 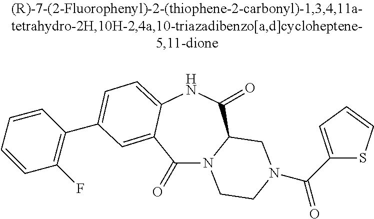 |
| “B24” | 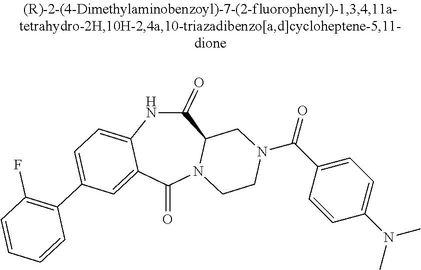 |
| “B25” | 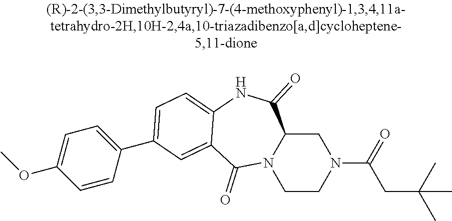 |
| “B26” | 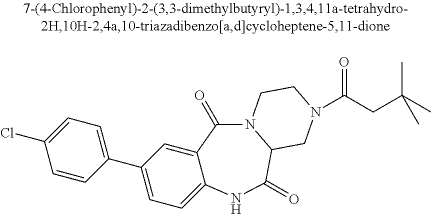 |
| “B27” | 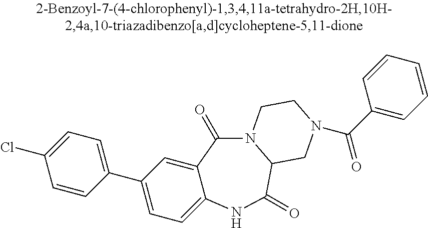 |

are excluded.

2. Compounds according to claim 1 in which
R² denotes H, (CH₂)ₙ₃NH₂, (CH₂)ₙ₃NHA, (CH₂)ₙ₃NA₂, (CH₂)ₙ₃OH, (CH₂)ₙ₃OA, (CH₂)ₙ₃Het², CH₂COHet², CH₂CONH₂, CH₂CONHA, CH₂CONA₂ or methyl,
and pharmaceutically usable salts and stereoisomers thereof, including mixtures thereof in all ratios.

3. Compounds according to claim 1 in which
X, X₁ each, independently of one another, denote CO or CH₂,
and pharmaceutically usable salts and stereoisomers thereof, including mixtures thereof in all ratios.

4. Compounds according to claim 1 in which
Y, Y₁ denote CH,
and pharmaceutically usable salts and stereoisomers thereof, including mixtures thereof in all ratios.

5. Compounds according to claim 1 in which
A denotes unbranched or branched alkyl having 1-10 C atoms,
in which 1-7H atoms may be replaced by F and/or Cl,
or
cyclic alkyl having 3-7 C atoms,
and pharmaceutically usable salts and stereoisomers thereof, including mixtures thereof in all ratios.

6. Compounds according to claim 1 in which
Ar denotes phenyl which is unsubstituted or mono-, di- or trisubstituted by Hal, A, OH, OA, NH₂, NHA and/or NA₂,
and pharmaceutically usable salts and stereoisomers thereof, including mixtures thereof in all ratios.

7. Compounds according to claim 1 in which
Het denotes a mono-, bi- or tricyclic saturated, unsaturated or aromatic heterocycle having 1 to 4 N, O and/or S atoms, which may be unsubstituted or mono-, di- or trisubstituted by A, Het², OH, NH₂, NHA and/or NA₂,
and pharmaceutically usable salts and stereoisomers thereof, including mixtures thereof in all ratios.

8. Compounds according to claim 1 in which
Het¹ denotes a mono-, bi- or tricyclic aromatic heterocycle having 1 to 4 N, O and/or S atoms, which may be unsubstituted or mono-, di- or trisubstituted by A and/or Hal,
and pharmaceutically usable salts and stereoisomers thereof, including mixtures thereof in all ratios.

9. Compounds according to claim 1 in which
R denotes Hal, Ar or Het',
R¹ denotes SO₂A, COOA, COOH, Cyc, Het, Ar, COHet, CONHHet, CONHAr, CHO, CONH₂, CONHA, CONA₂, (CH₂)ₙ₂OH, (CH₂)ₙ₂OA, OAr, NHAr, (CH₂)ₙ₂NH₂, (CH₂)ₙ₂NHA, (CH₂)ₙ₂NA₂ or A,
R² denotes H, (CH₂)ₙ₃NH₂, (CH₂)ₙ₃NHA, (CH₂)ₙ₃NA₂, (CH₂)ₙ₃OH, (CH₂)ₙ₃OA, (CH₂)ₙ₃Het², CH₂COHet², CH₂CONH₂, CH₂CONHA, CH₂CONA₂ or methyl,
X, X₁ each, independently of one another, denote CO or CH₂,
Y, Y₁ denote CH,
Q denotes CO, SO₂ or COO,
E denotes CO, CH(OH), CA(OH), CH(OA), CA(OA), CH(NH₂), Alk, 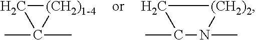
Alk denotes linear or branched alkylene having 1-8 C atoms, in which one or two CH₂ groups may be replaced by 0 and/or NH,
n1 denotes 0, 1 or 2,
n2 denotes 0, 1, 2, 3 or 4,
n3 denotes 1, 2, 3 or 4,
Ar denotes phenyl which is unsubstituted or mono-, di- or trisubstituted by Hal, A, OH, OA, NH₂, NHA and/or NA₂,
Het denotes a mono-, bi- or tricyclic saturated, unsaturated or aromatic heterocycle having 1 to 4 N, O and/or S atoms, which may be unsubstituted or mono-, di- or trisubstituted by A, Het², OH, NH₂, NHA and/or NA₂,
Het¹ denotes a mono-, bi- or tricyclic aromatic heterocycle having 1 to 4 N, O and/or S atoms, which may be unsubstituted or mono-, di- or trisubstituted by A and/or Hal,
Het² denotes pyrrolidinyl, piperidinyl, thiazolidinyl, morpholinyl, oxazolidinyl, tetrahydroquinazolinyl, tetrahydropyranyl, piperazinyl, thiazolyl, furyl, thienyl, pyrrolyl, imidazolyl, pyrazolyl, oxazolyl, isoxazolyl, isothiazolyl, pyridyl, pyrimidinyl, triazolyl, tetrazolyl, oxadiazolyl or thiadiazolyl, each of which is unsubstituted or monosubstituted by A,
Cyc denotes cyclic alkyl having 3-7 C atoms,
A denotes unbranched or branched alkyl having 1-10 C atoms,
in which 1-7H atoms may be replaced by F and/or Cl,
or
cyclic alkyl having 3-7 C atoms,
Hal denotes F, Cl, Br or I.

10. Compounds according to claim 1 selected from the group

| Compound No. | Name and/or structure |
|---|---|
| “10” | 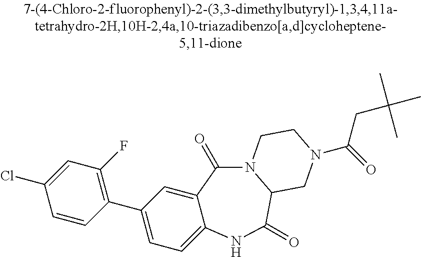 |
| “17” | 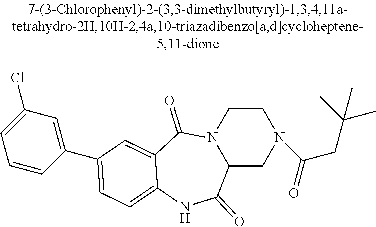 |
| “25” | 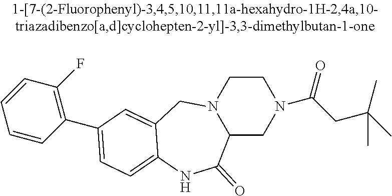 |
| “26” | 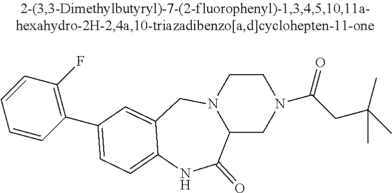 |
| “28” | 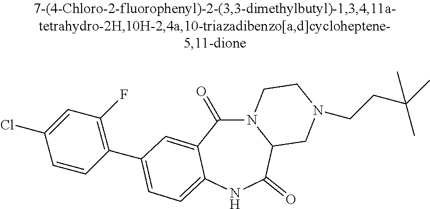 |
| “31” | 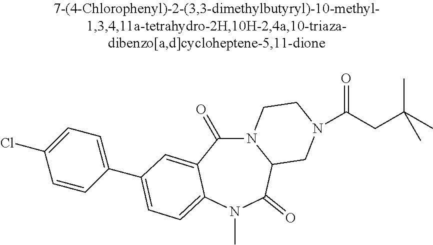 |
| “37” | 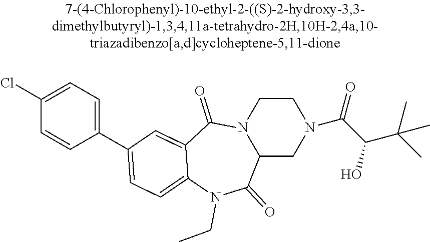 |
| “A1” | 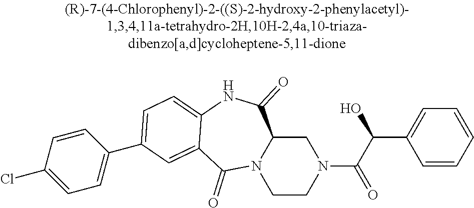 |
| “A2” | 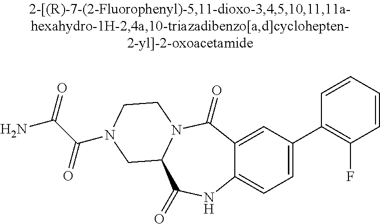 |
| “A3” | 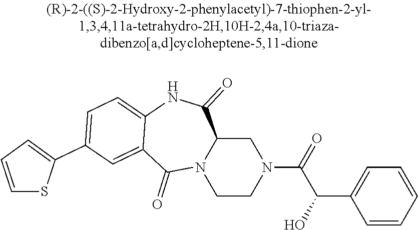 |
| “A4” | 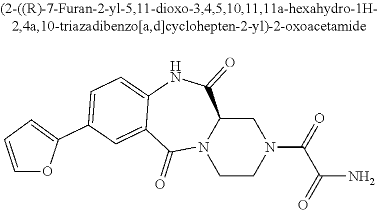 |
| “A5” | 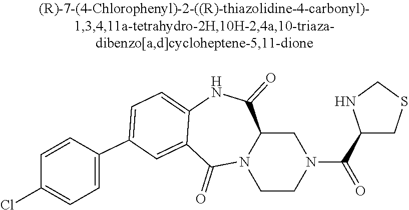 |
| “A6” | 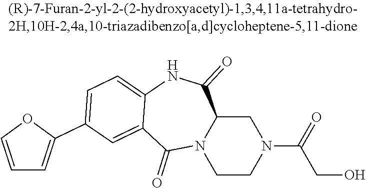 |
| “A7” | 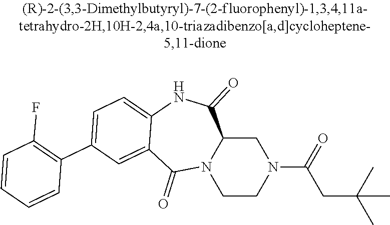 |
| “A8” | 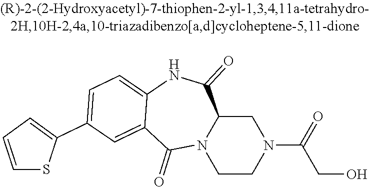 |
| “A9” | 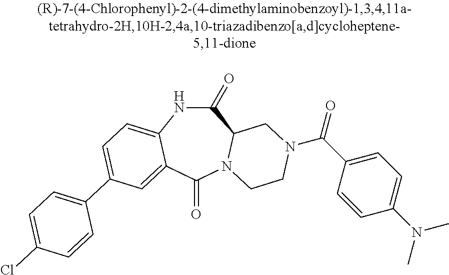 |
| “A10” | 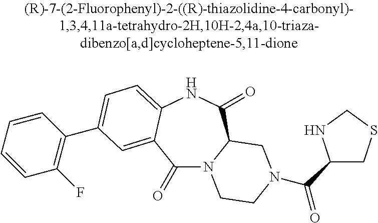 |
| “A11” | 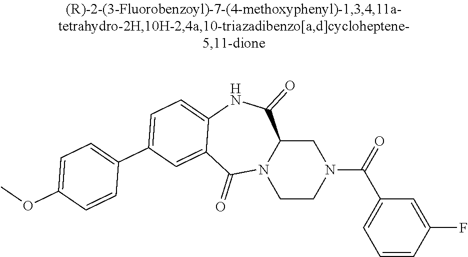 |
| “A12” | 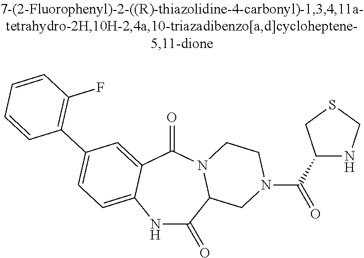 |
| “A13” | 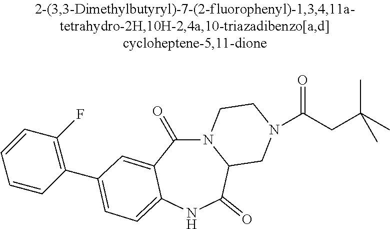 |
| “A14” | 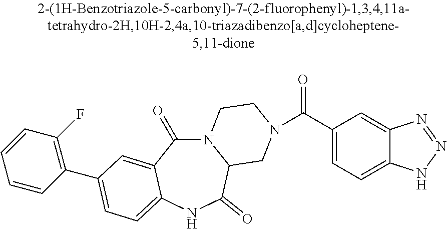 |
| “A15” | 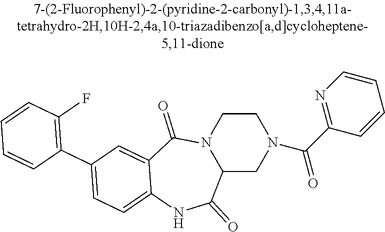 |
| “A16” | 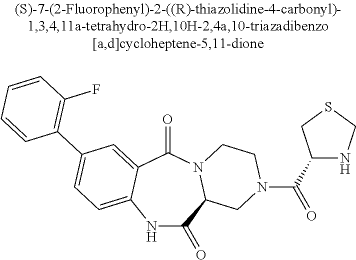 |
| “A17” | 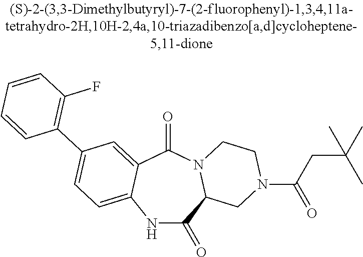 |
| “A18” | 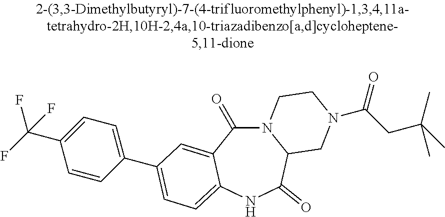 |
| “A19” | 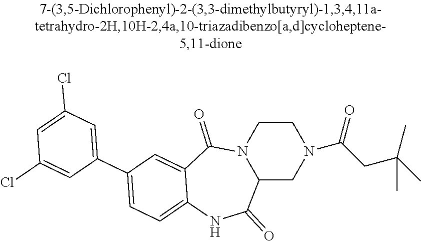 |
| “A20” | 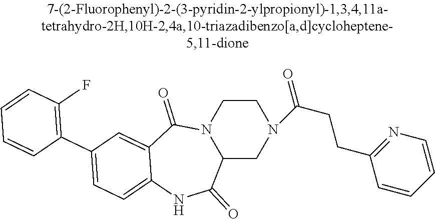 |
| “A21” | 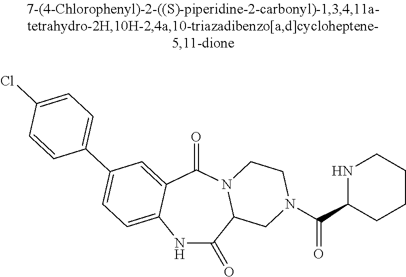 |
| “A22” | 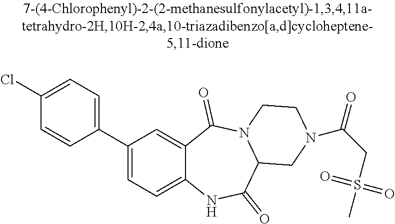 |
| “A23” | 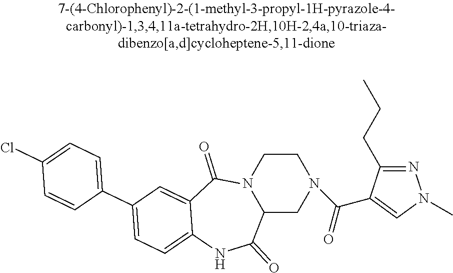 |
| “A24” | 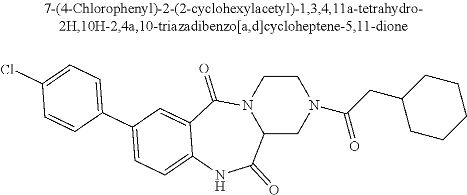 |
| “A25” | 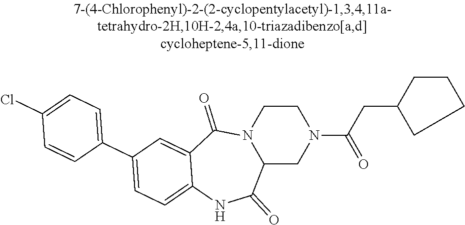 |
| “A26” | 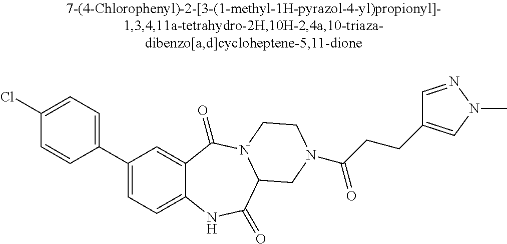 |
| “A27” | 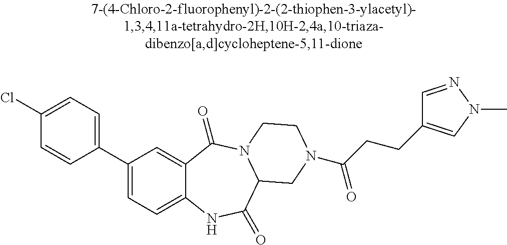 |
| “A28” | 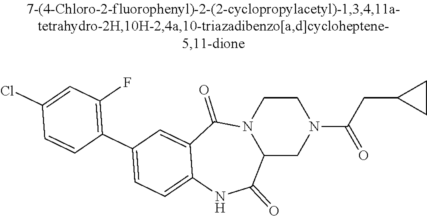 |
| “A29” | 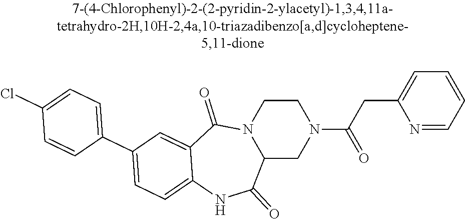 |
| “A30” | 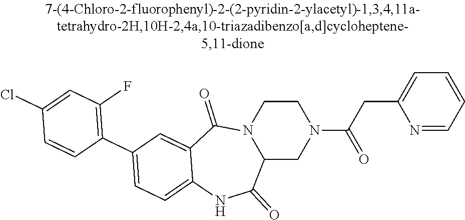 |
| “A31” | 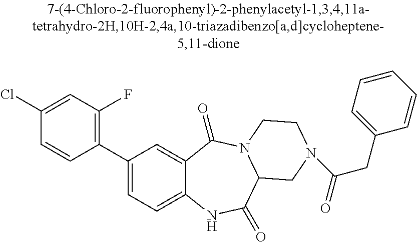 |
| “A32” | 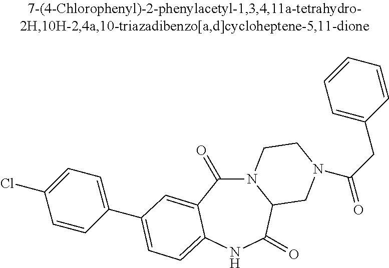 |
| “A33” | 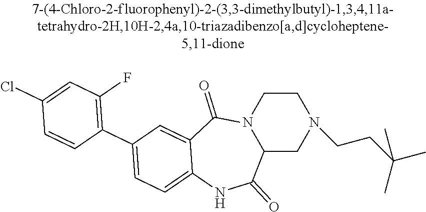 |
| “A34” | 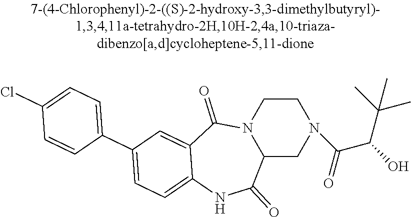 |
| “A35” | 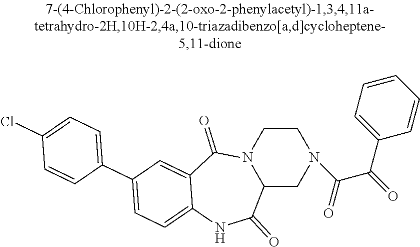 |
| “A36” | 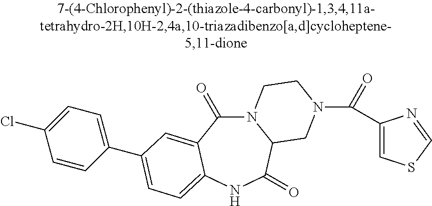 |
| “A37” | 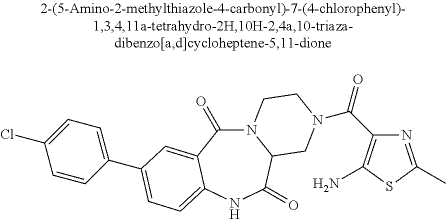 |
| “A38” | 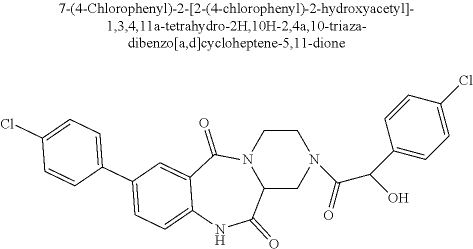 |
| “A39” | 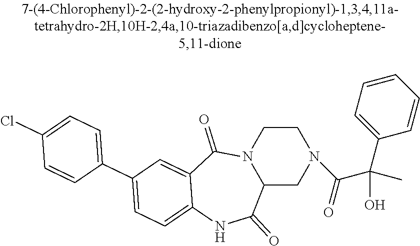 |
| “A40” | 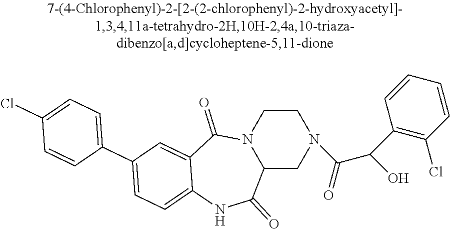 |
| “A41” | 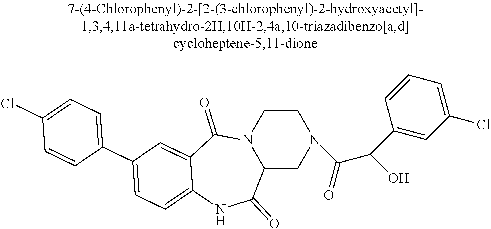 |
| “A42” | 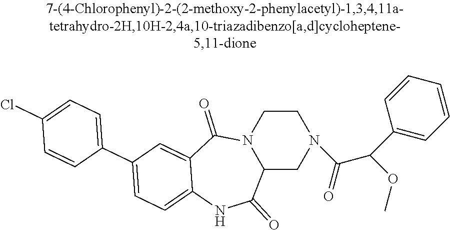 |
| “A43” | 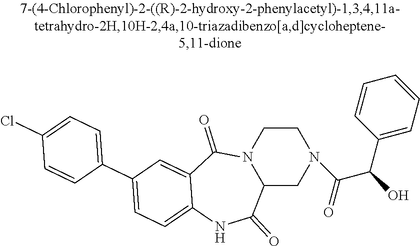 |
| “A44” | 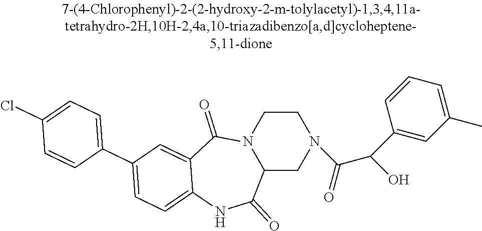 |
| “A45” | 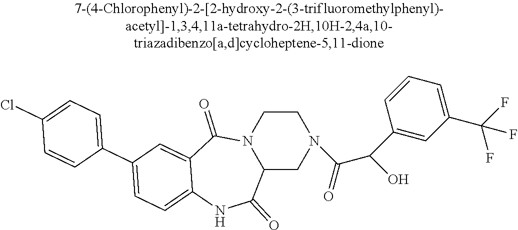 |
| “A46” | 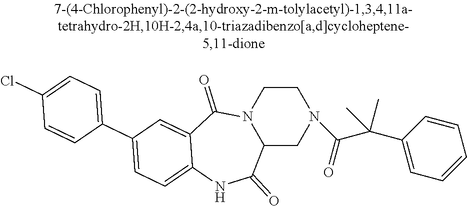 |
| “A47” | 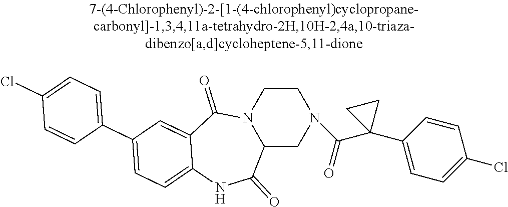 |
| “A48” | 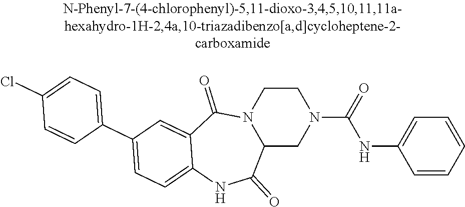 |
| “A49” | 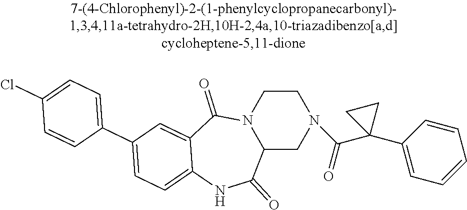 |
| “A50” | 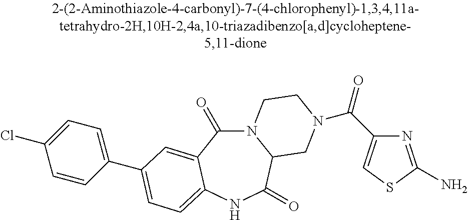 |
| “A51” | 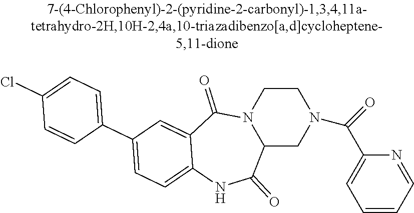 |
| “A53” | 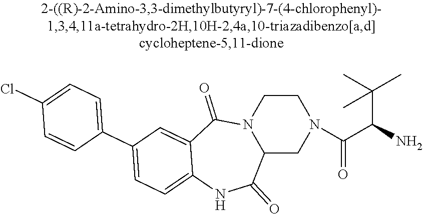 |
| “A54” | 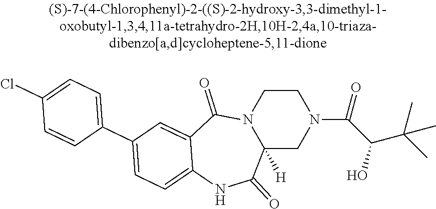 |
| “A55” | 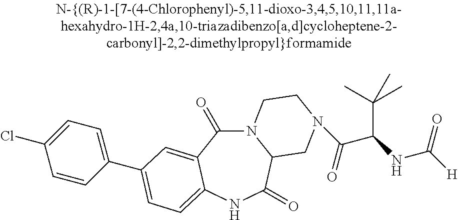 |
| “A56” | 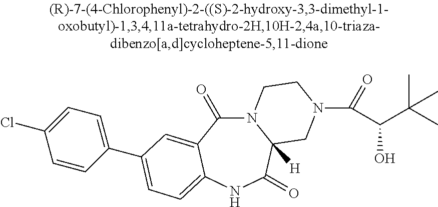 |
| “A57” | 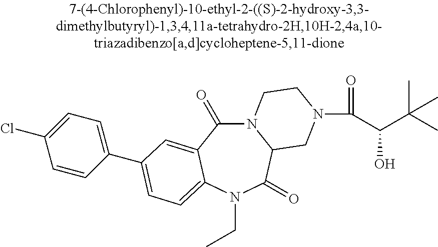 |
| “40” | 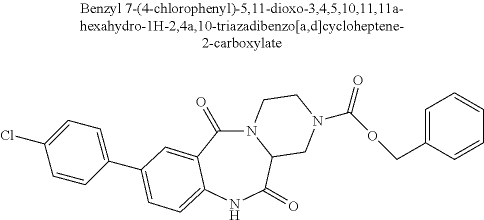 |
| “41” | 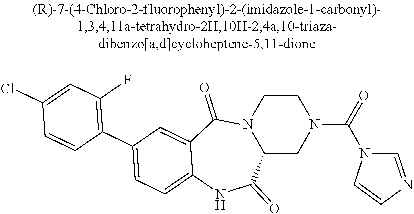 |
| “48” | 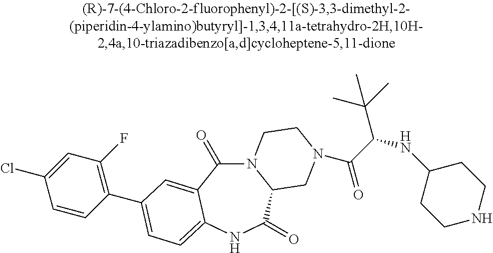 |
| “50” | 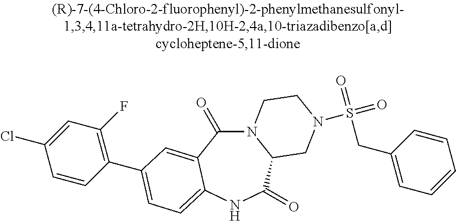 |
| “57” | 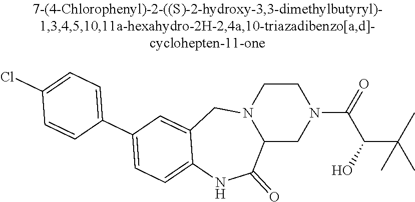 |
| “A58” | 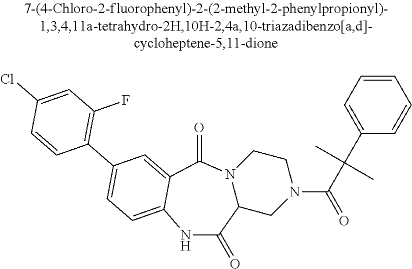 |
| “A59” | 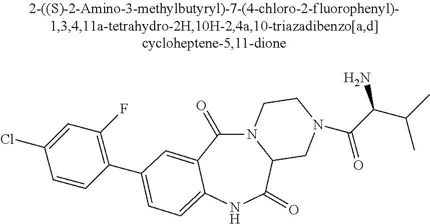 |
| “A60” | 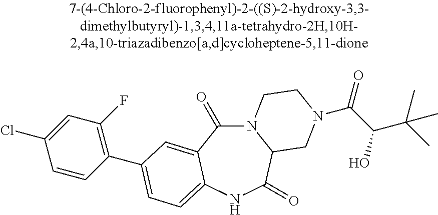 |
| “A61” | 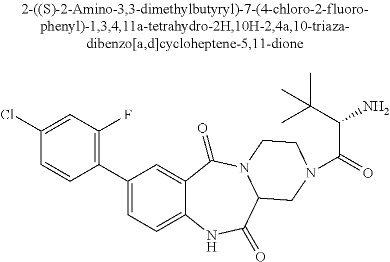 |
| “A62” | 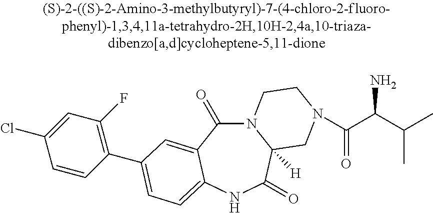 |
| “A63” | 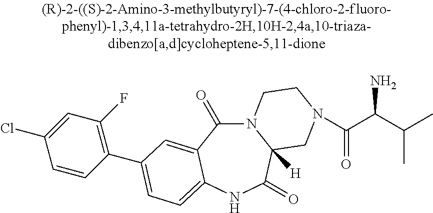 |
| “A64” | 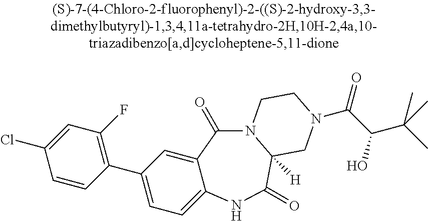 |
| “A65” | 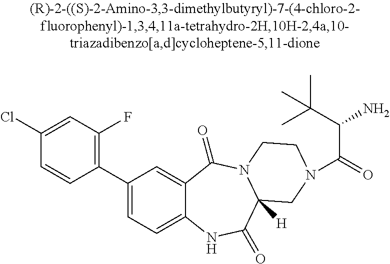 |
| “A66” | 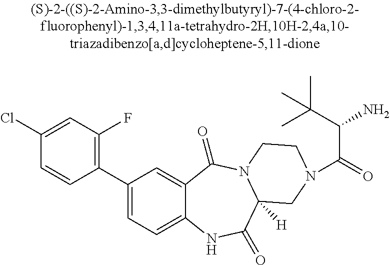 |
| “A67” | 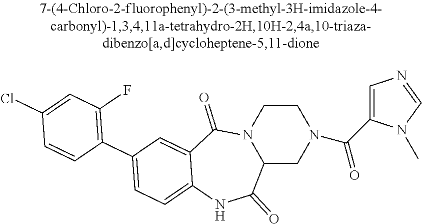 |
| “A68” | 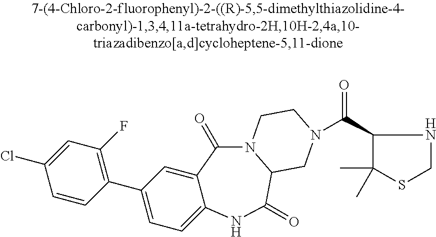 |
| “A69” | 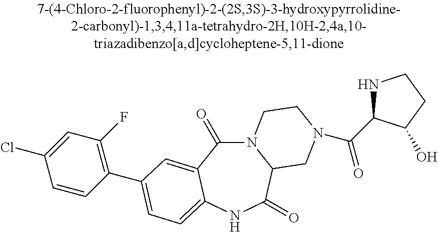 |
| “A70” | 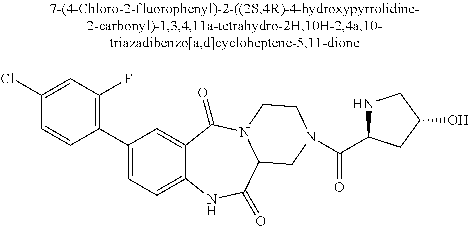 |
| “A71” | 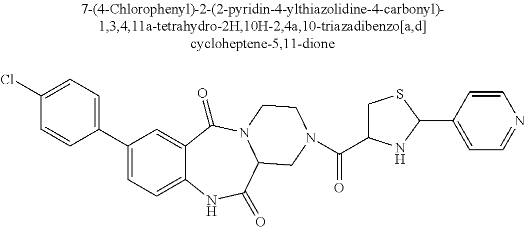 |
| “A72” | 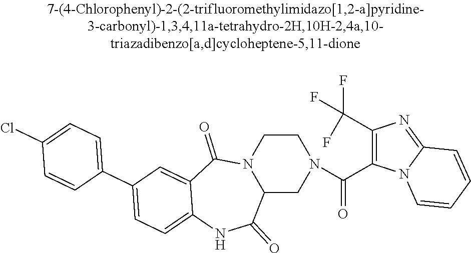 |
| “A73” | 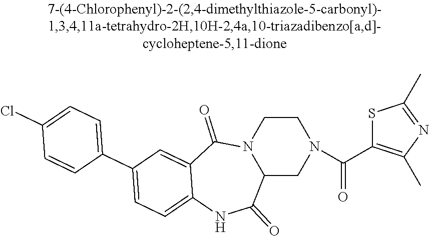 |
| “A74” | 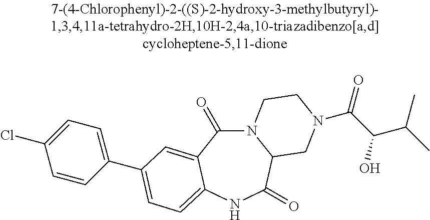 |
| “A75” | 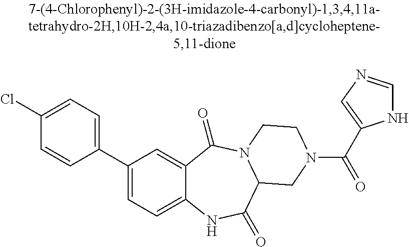 |
| “A76” | 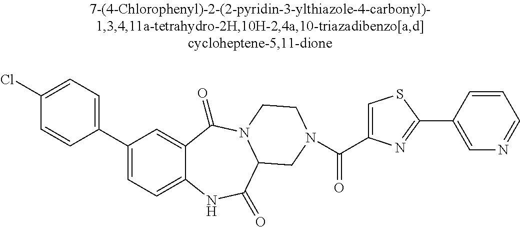 |
| “A77” | 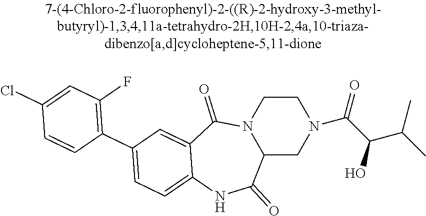 |
| “A78” | 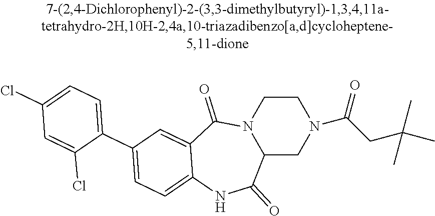 |
| “A79” | 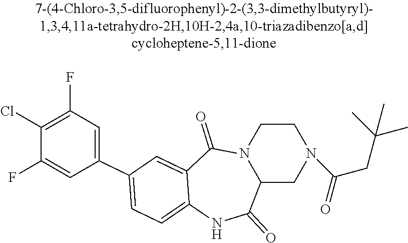 |
| “A80” | 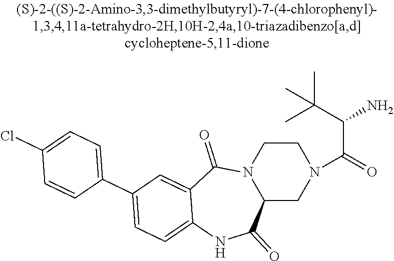 |
| “A81” | 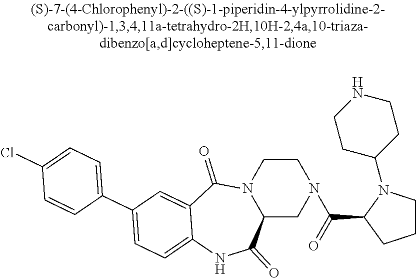 |
| “A82” | 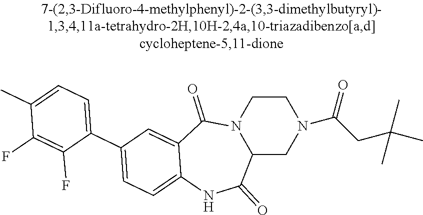 |
| “A83” | 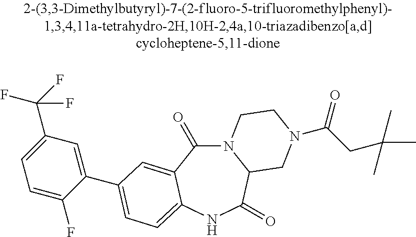 |
| “A84” | 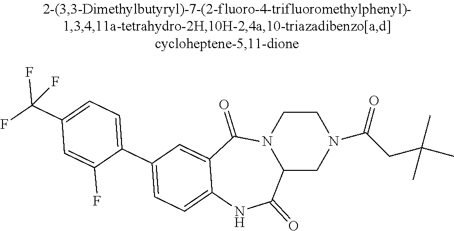 |
| “A85” | 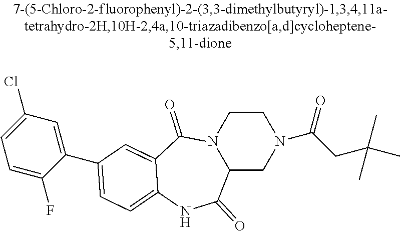 |
| “A86” | 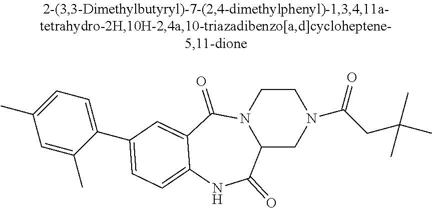 |
| “A87” | 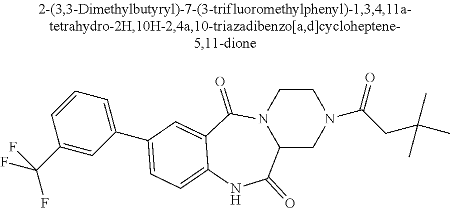 |
| “A88” | 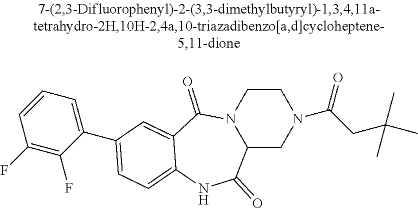 |
| “A89” | 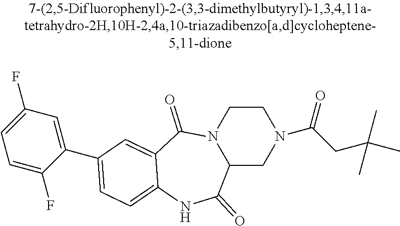 |
| “A90” | 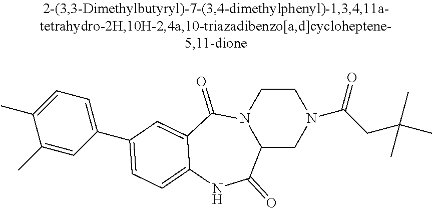 |
| “A91” | 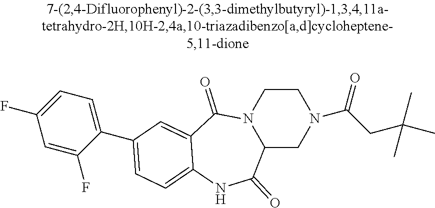 |
| “A92” | 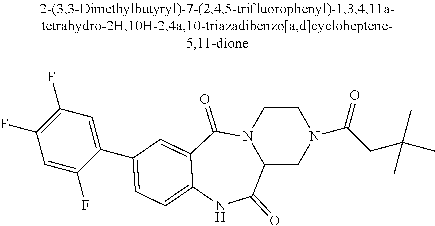 |
| “A93” | 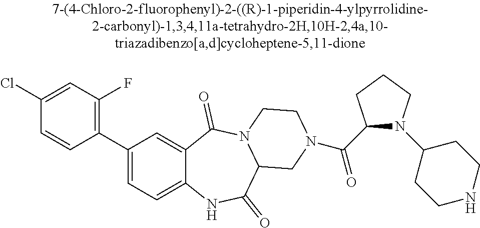 |
| “A94” | 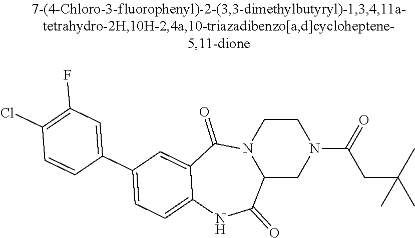 |
| “A95” | 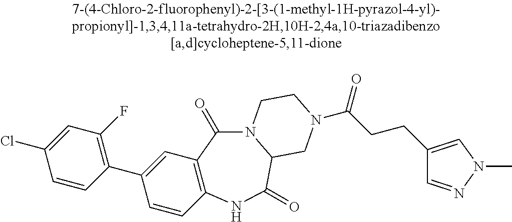 |
| “A96” | 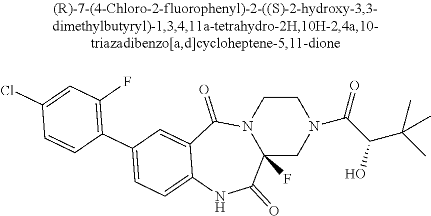 |
| “A97” | 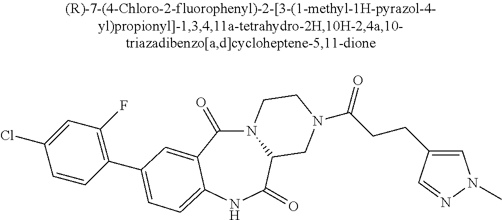 |
| “A98” | 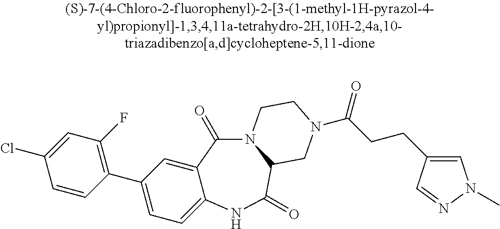 |
| “A99” | 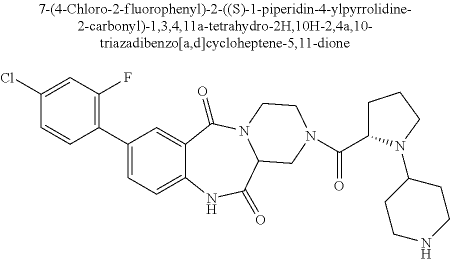 |
| “A100” | 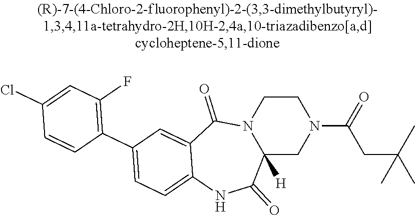 |
| “A101” | 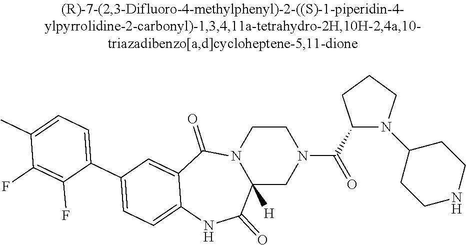 |
| “A102” | 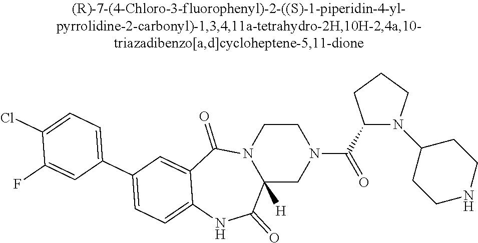 |
| “A103” | 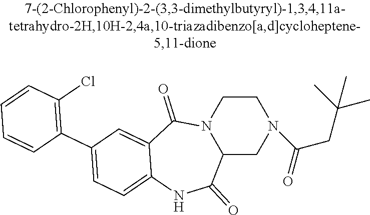 |
| “A104” | 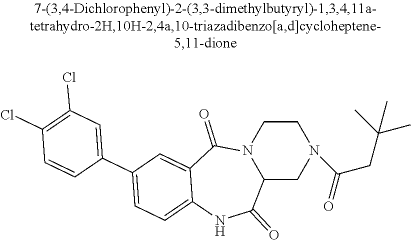 |
| “A105” | 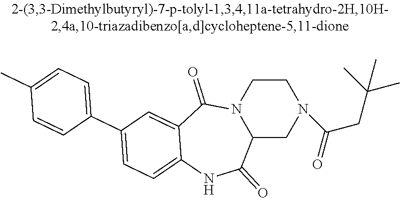 |
| “A106” | 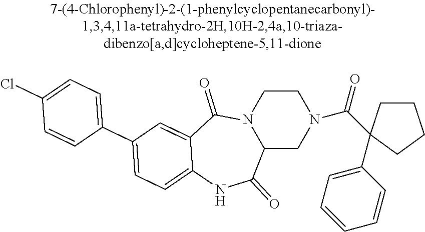 |
| “A107” | 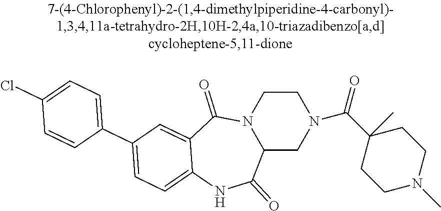 |
| “A108” | 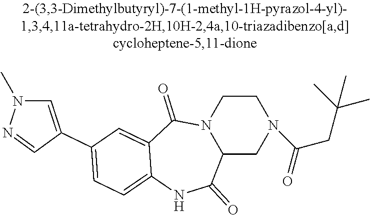 |
| “A109” | 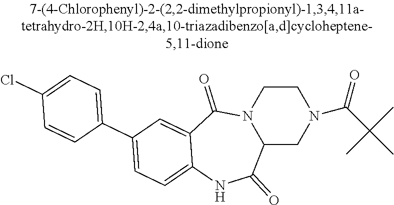 |
| “A110” | 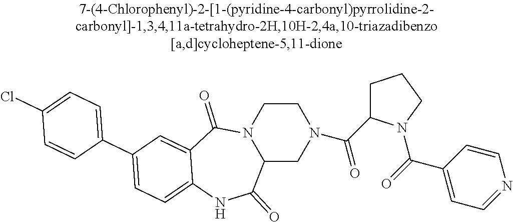 |
| “A111” | 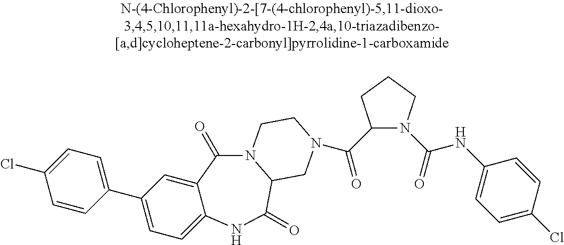 |
| “A112” | 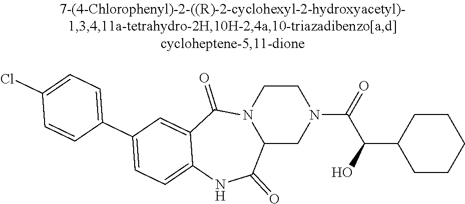 |
| “A113” | 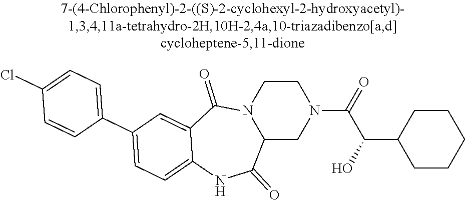 |
| “A114” | 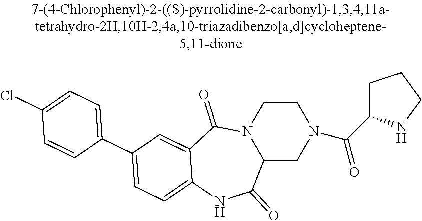 |
| “A115” | 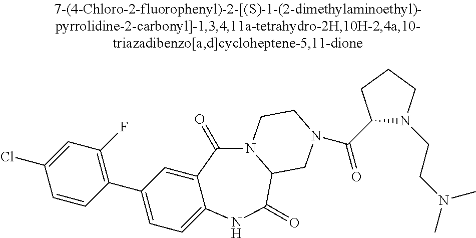 |
| “A116” | 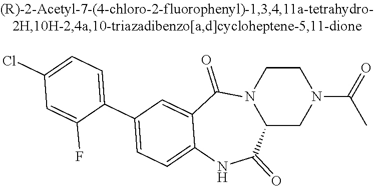 |
| “A117” | 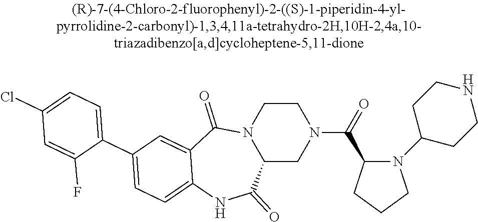 |
| “A118” | 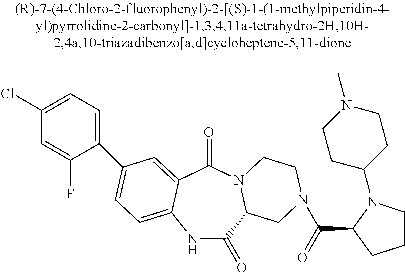 |
| “A119” | 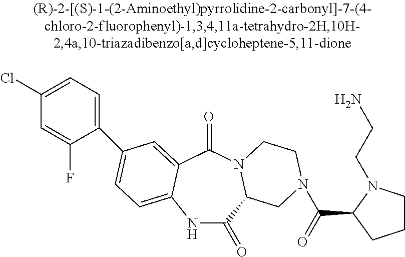 |
| “A120” | 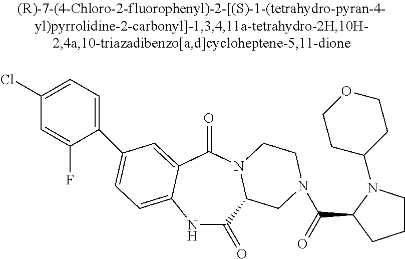 |
| “A121” | 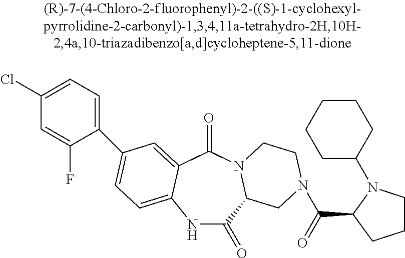 |
| “A122” | 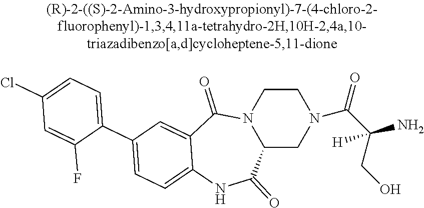 |
| “A123” | 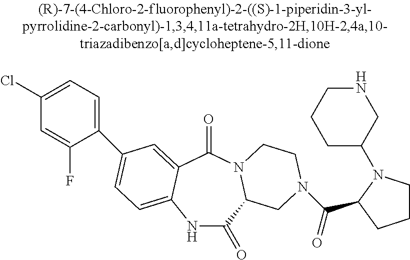 |
| “A124” | 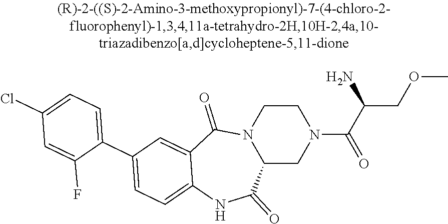 |
| “A125” | 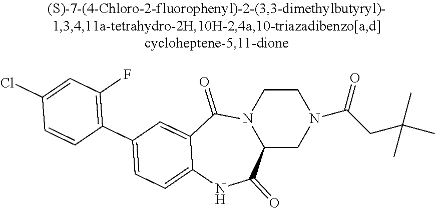 |
| “A126” | 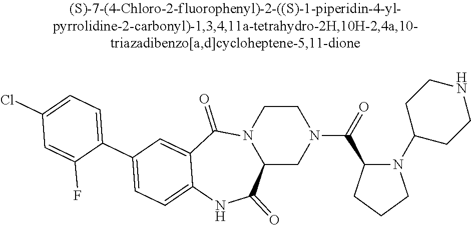 |
| “A128” | 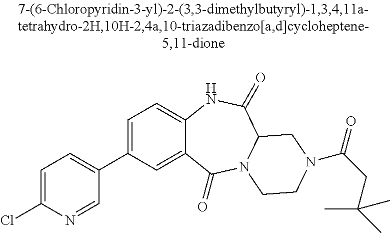 |
| “A129” | 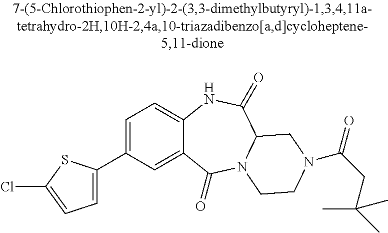 |
| “A130” | 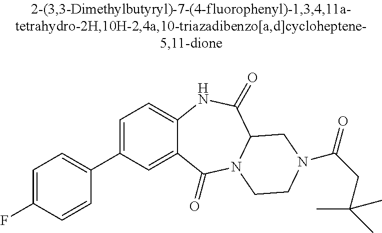 |
| “A131” | 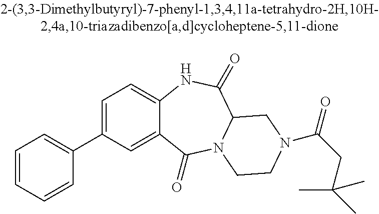 |
| “A132” | 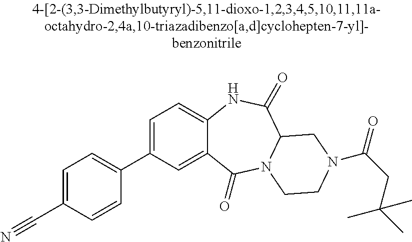 |
| “A133” | 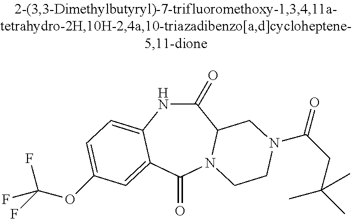 |
| “A134” | 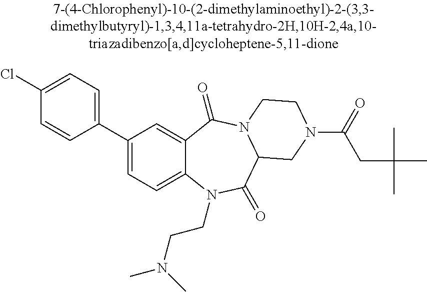 |
| “A135” | 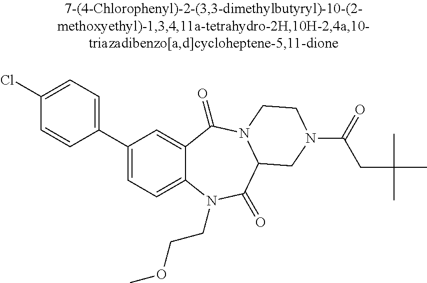 |
| “A136” | 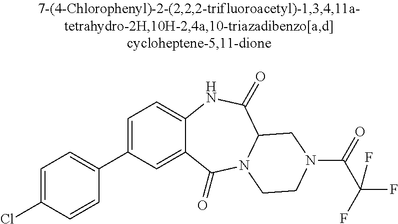 |
| “A137” | 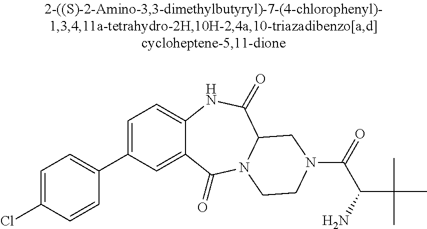 |
| “A138” | 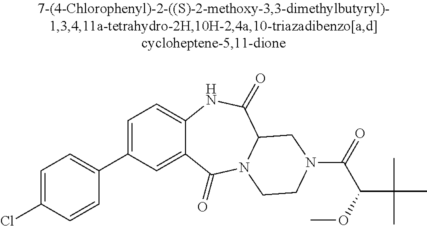 |
| “A139” | 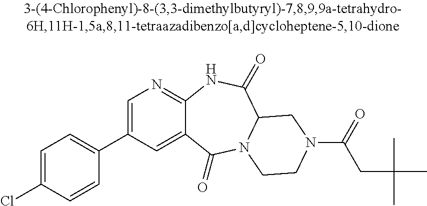 |
| “A140” | 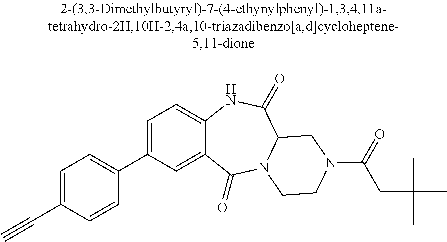 |
| “A141” | 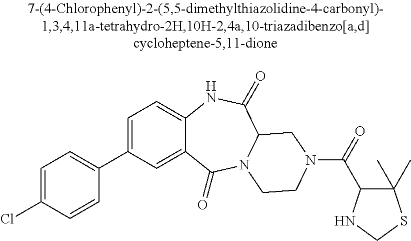 |
| “A142” | 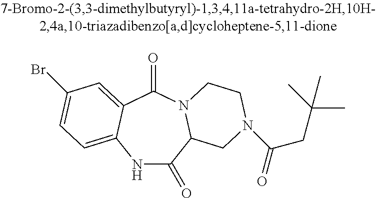 |
| “A143” | 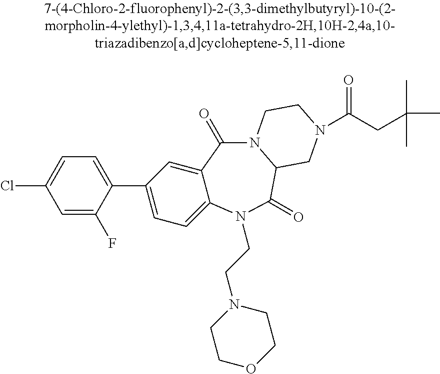 |
| “A144” | 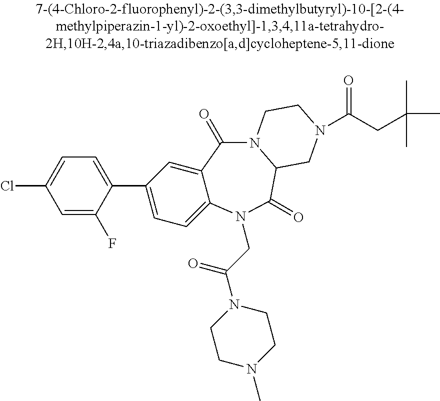 |
| “A145” | 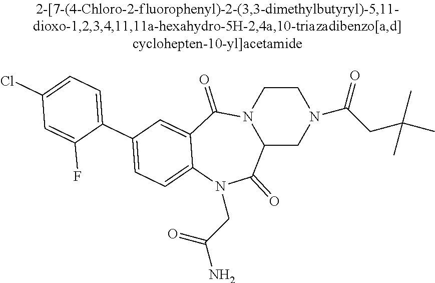 |
| “A146” | 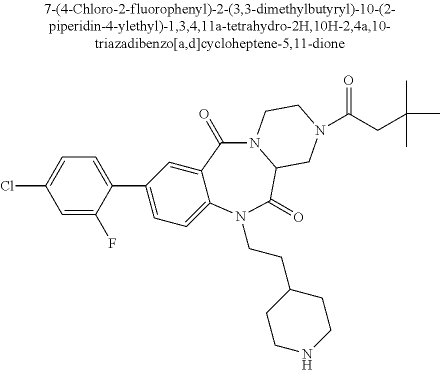 |
| “A147” | 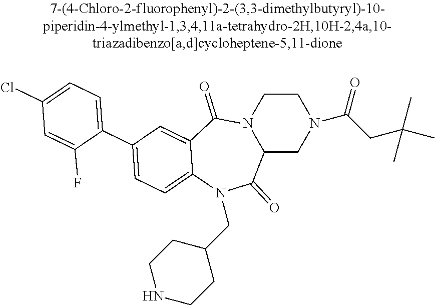 |
| “A148” | 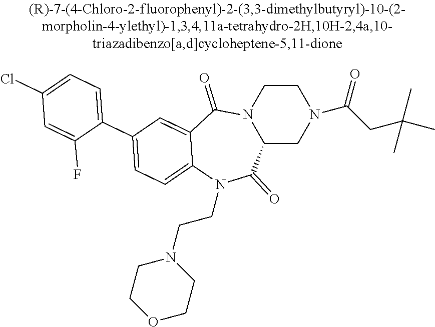 |
| “A149” | 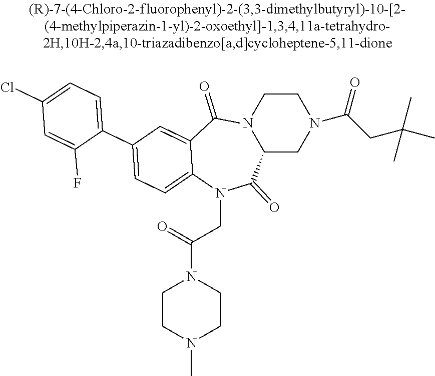 |
| “A150” | 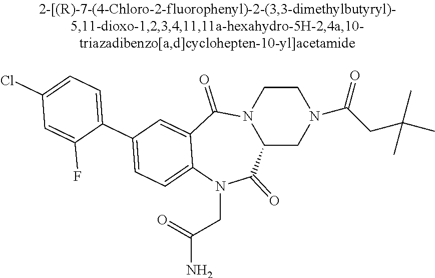 |
| “A151” | 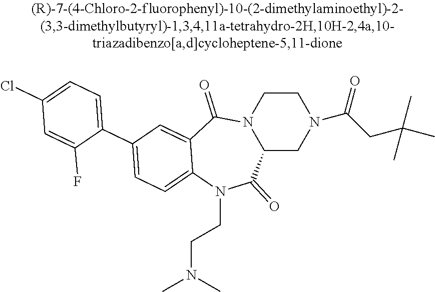 |
| “A152” | 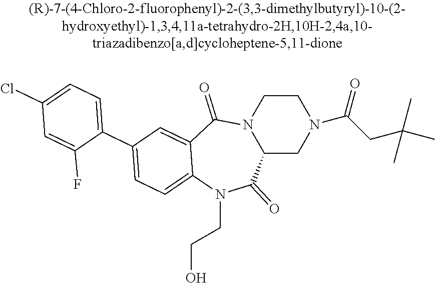 |
| “A153” | 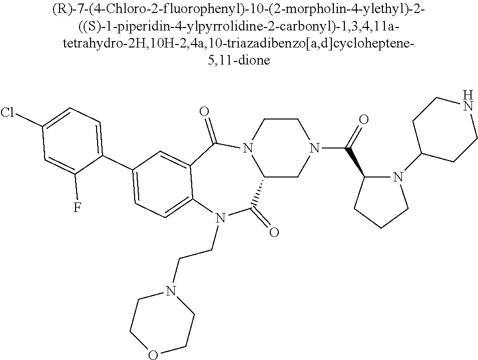 |
| “A154” | 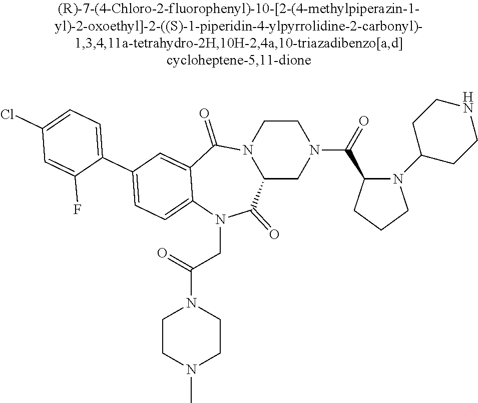 |
| “A155” | 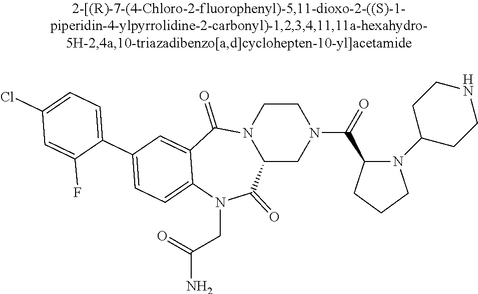 |
| “A156” | 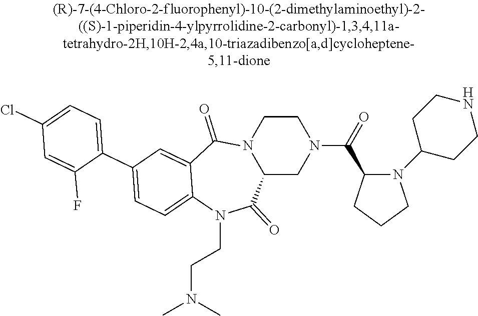 |
| “A157” | 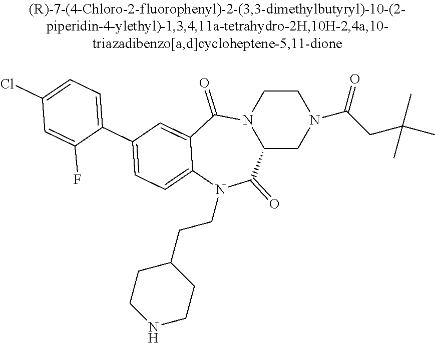 |
| “A158” | 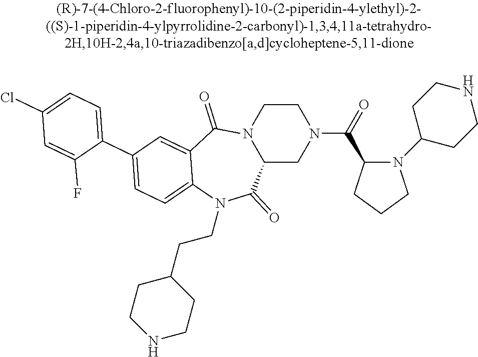 |
| “A159” | 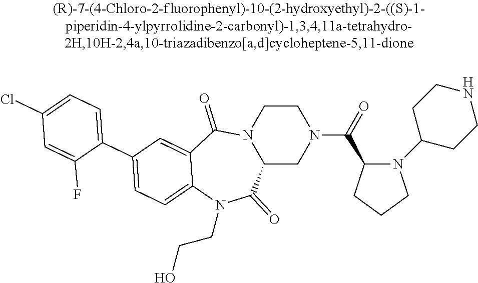 |
| “A160” | 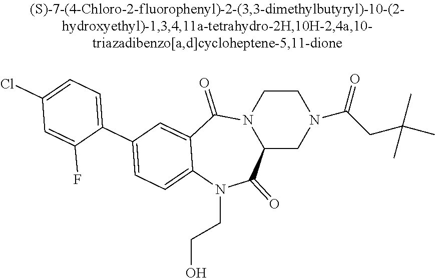 |
| “A161” | 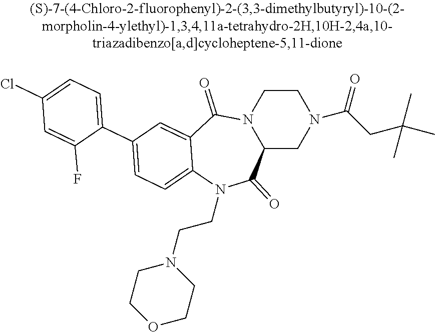 |
| “A162” | 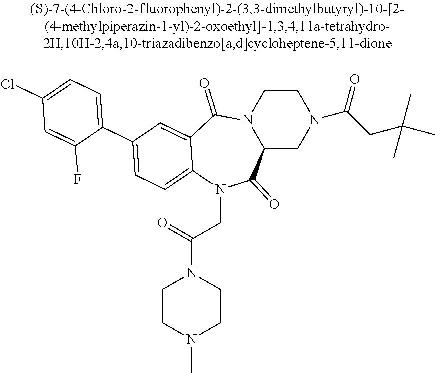 |
| “A163” | 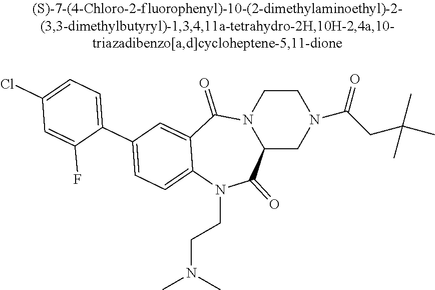 |
| “A164” | 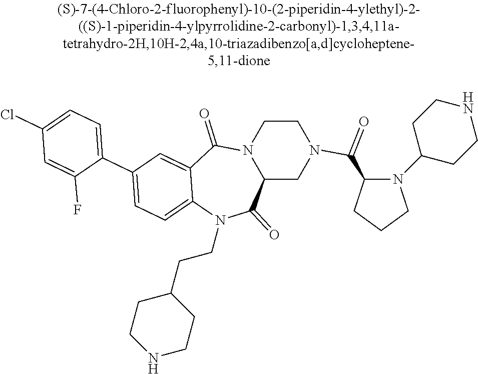 |
| “A165” | 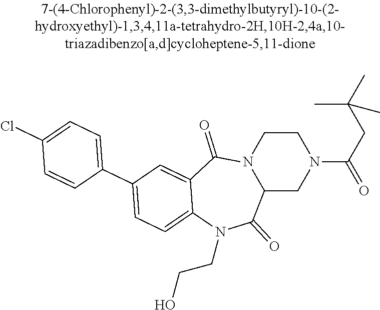 |
| “A166” | 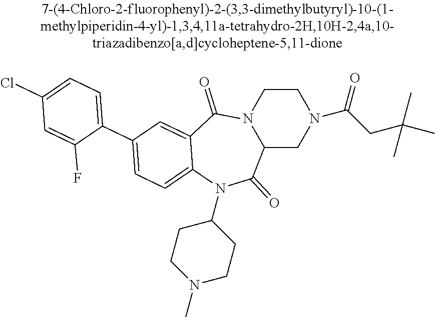 |
| “A167” | 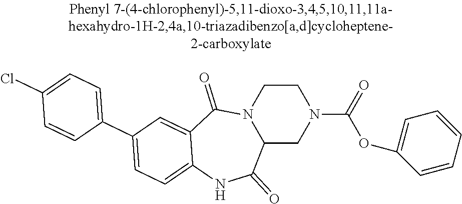 |
| “A168” | 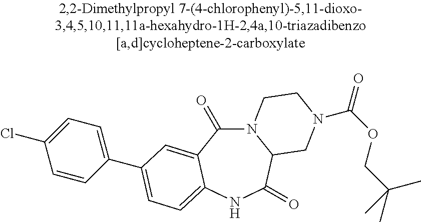 |
| “A170” | 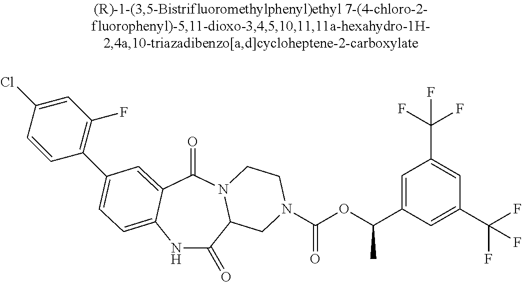 |
| “A173” | 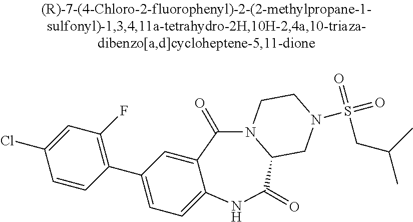 |
| “A174” | 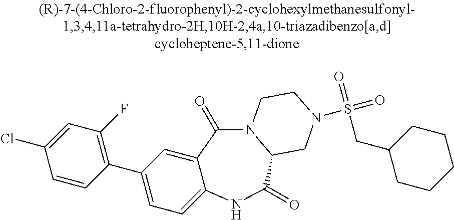 |
| “A176” | 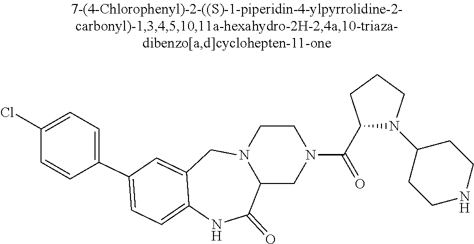 |

and pharmaceutically usable salts and stereoisomers thereof, including mixtures thereof in all ratios.

11. Medicaments comprising at least one compound of the formula I according to claim 1 or a compound “B1”-“B27” and/or pharmaceutically usable salts and stereoisomers thereof, including mixtures thereof in all ratios, and optionally excipients and/or adjuvants.

12. Compounds of the formula I and the compounds “B1”-“B27” and pharmaceutically usable salts and stereoisomers thereof, including mixtures thereof in all ratios,
for treatment and/or prophylaxis of tumours, tumour diseases and cancer diseases.

13. Compounds according to claim 12, where the cancer diseases are selected from the group of tumours of the squamous epithelium, of the bladder, of the stomach, of the kidneys, of head and neck, of the oesophagus, of the cervix, of the thyroid, of the intestine, of the liver, of the brain, of the prostate, of the urogenital tract, of the lymphatic system, of the stomach, of the larynx and/or of the lung.

14. Compounds according to claim 12, where the tumour originates from the group monocytic leukaemia, lung adenocarcinoma, small-cell lung carcinomas, pancreatic cancer, ovarian carcinoma, glioblastomas and breast carcinoma and colon carcinoma.

15. Compounds according to claim 12, where the disease to be treated is a tumour of the blood and immune system.

16. Compounds according to claim 12, where the tumour originates from the group of acute myeloid leukaemia, chronic myeloid leukaemia, acute lymphatic leukaemia and/or chronic lymphatic leukaemia.

17. The compound 4′-chloro-4-({(R)-4-[(S)-3,3-dimethyl-2-(piperidin-4-ylamino)butyryl]piperazin-2-carbonyl}amino)-2′-fluorobiphenyl-3-carboxylic acid (“47”) and salts thereof.
